(19) United States
(12) Patent Application Publication
(10) Pub. No.: US 2022/0062291 A1
(43) Pub. Date: Mar. 3, 2022

(54) COMPOSITIONS AND METHODS OF TREATING CANCERS BY ADMINISTERING A PHENOTHIAZINE-RELATED DRUG THAT ACTIVATES PROTEIN PHOSPHATASE 2A (PP2A) WITH REDUCED INHIBITORY ACTIVITY TARGETED TO THE DOPAMINE D2 RECEPTOR AND ACCOMPANYING TOXICITY

(71) Applicant: DANA-FARBER CANCER INSTITUTE, INC., Boston, MA (US)

(72) Inventors: Alfred T. Look, North Reading, MA (US); Ken Morita, Boston, MA (US); Eric S. Fischer, Chestnut Hill, MA (US); Nathanael S. Gray, Boston, MA (US); Shuning He, Boston, MA (US)

(73) Assignee: DANA-FARBER CANCER INSTITUTE, INC., Boston, MA (US)

(21) Appl. No.: 17/415,429

(22) PCT Filed: Dec. 19, 2019
(86) PCT No.: PCT/US19/67508

§ 371 Date: Jun. 17, 2021

Related U.S. Application Data
(60) Provisional application No. 62/846,028, filed on May 10, 2019.
(60) Provisional application No. 62/783,959, filed on Dec. 21, 2018.

(51) Int. Cl. A61K 31/5415; A61K 31/538; A61K 45/06; A61P 7/00

(52) U.S. Cl. CPC A61K 31/5415; A61P 7/00; A61K 45/06; A61K 31/538

(57) ABSTRACT

Disclosed are compositions and methods of treating cancers by constitutively activating protein phosphatase 2A (PP2A) without blocking signaling through the dopamine D2 receptor, that entail administering a therapeutically effective amount of an analog of perphenazine (PPZ) of formula (I) or (II), or a related PPZ analog lacking dopamine receptor D2 inhibitory activity, or a pharmaceutically acceptable salt thereof.

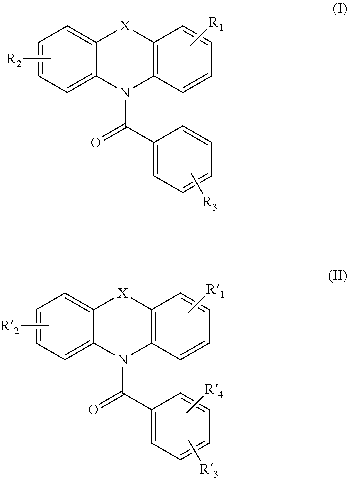

### RELATED APPLICATIONS

This application claims the benefit of priority under 35 U.S.C. § 119(e) to U.S. Provisional Application No. 62/783,959, filed on Dec. 21, 2018 and to U.S. Provisional Application No. 62/846,028, filed on May 10, 2019, each of which is incorporated herein by reference in its entirety.

### GOVERNMENT LICENSE RIGHTS

This invention was made with government support under grant numbers R35 CA210064, R01 CA214608, and R01 CA218278 awarded by the National Institutes of Health. The government has certain rights in the invention.

### BACKGROUND OF THE INVENTION

Phenothiazines have been used for over 50 years as neuroleptic-type antipsychotic medications. The antipsychotic effects of phenothiazines correlate with their ability to block dopamine receptors, but a broad array of other activities have been described, including antitumor effects.

PPZ and its analogs activate protein phosphatase 2A (PP2A), a serine-threonine phosphatase enzyme that removes activating phosphates from AKT, ERK, KRAS, MYC and other oncoproteins that are predominant oncogenic drivers of pathways and dependencies in many types of cancer.

T-cell acute lymphoblastic leukemia (T-ALL) is an aggressive malignancy of early T-cell precursors arising in the thymus. T-cell acute lymphoblastic leukemia (T-ALL) accounts for about 15% and 25% of ALL in pediatric and adult cohorts, respectively (Chiaretti and Foa, Haematologica 94(2):160-162 (2009)). Intensified treatment regimens have improved outcomes, but patients who fail conventional therapy have a dismal prognosis, and T-ALL remains fatal in 20% of children and more than 50% of adults (Goldberg et al., J . Clin. Oncol. 19:3616-22 (2003); Marks et al., Blood 114(25):5136-45 (2009); Ko et al., J. Clin. Oncol. 28(4):648-54 (2010)).

T-ALL cell lines treated with the antipsychotic drug perphenazine (PPZ) exhibited rapid dephosphorylation of multiple PP2A substrates and subsequent apoptosis. Moreover, shRNA knockdown of specific PP2A subunits attenuated PPZ activity, indicating that PP2A mediates the drug's antileukemic activity. It has been further reported that human T-ALLs treated with PPZ exhibited suppressed cell growth and dephosphorylation of PP2A targets in vitro and in vivo. (See, Gutierrez et al., J. Clin. Invest. 124(2):644-55 (2014)). However, PPZ also inhibits the dopamine D2 receptor (DRD2) in the basal ganglia, which causes movement disorders, including difficulty breathing and swallowing, thus posing a dose limiting effect of the drug. Thus, the propensity of PPZ to bind and inhibit dopamine receptors (e.g., DRD2) may lead to side effects at even low molar concentrations that may be substantially below the levels that are needed for PP2A activation and therapeutic activity against cancer.

### SUMMARY OF THE INVENTION

A first aspect of the present invention is directed to a method of treating a cancer, comprising administering to a subject in need thereof a therapeutically effective amount of a perphenazine (PPZ) analog which has a structure represented by formula I or II:

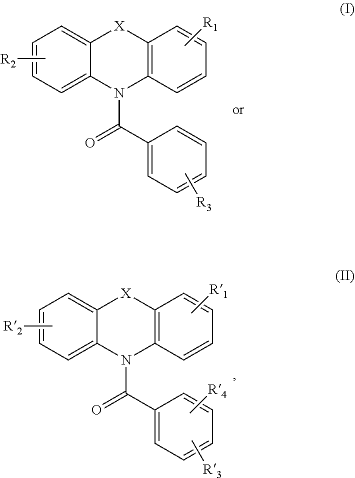

wherein X is O or S;
R₁ and R₂ are independently H, halo (e.g., Cl or F), NO₂ or CN;
R₃ is C1-C2 alkyl or methoxy;
R′₁ and R′₂ are independently H, halo, NO₂ or CN;
R′₃ and R′₄ independently halo, NO₂, CN, C1-C2 alkyl, methoxy, ethoxy, propoxy, isopropoxy, butoxy or benzyloxy; or R′₃ and R′₄ together with the atoms to which they are bound form a 6-membered aryl or 6-membered heteroaryl group,
or a pharmaceutically acceptable salt thereof.

In some embodiments, the compound of formula I or II has any one of the following structures:

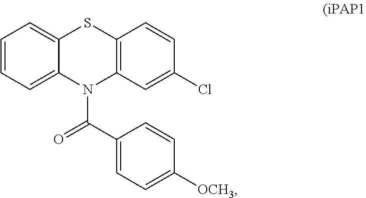 [SMILES: COc1ccc(C(=O)N2c3ccccc3Sc3ccc(Cl)cc32)cc1]

also known as iHAP1 (for improved heterocyclic activator of PP2A), Z56843374, P-889442 and 14B);

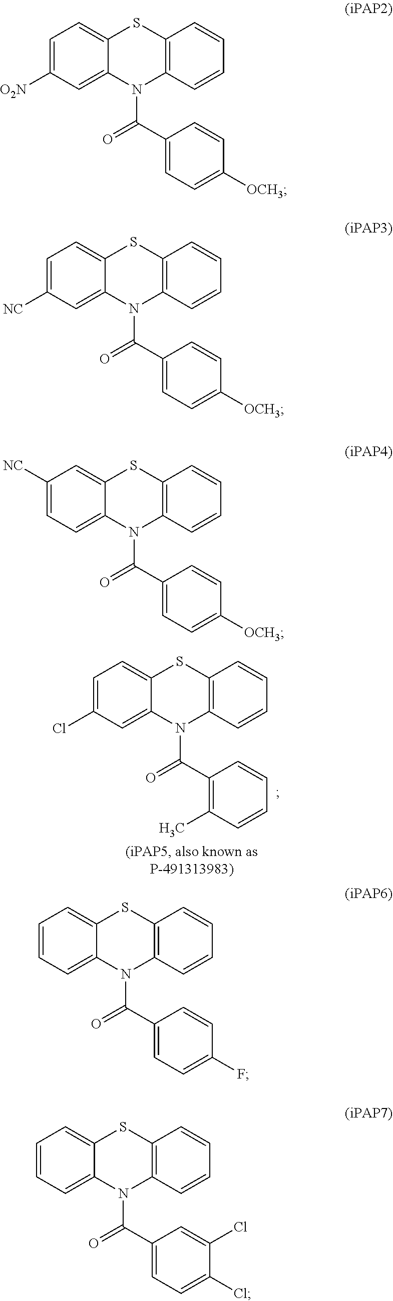

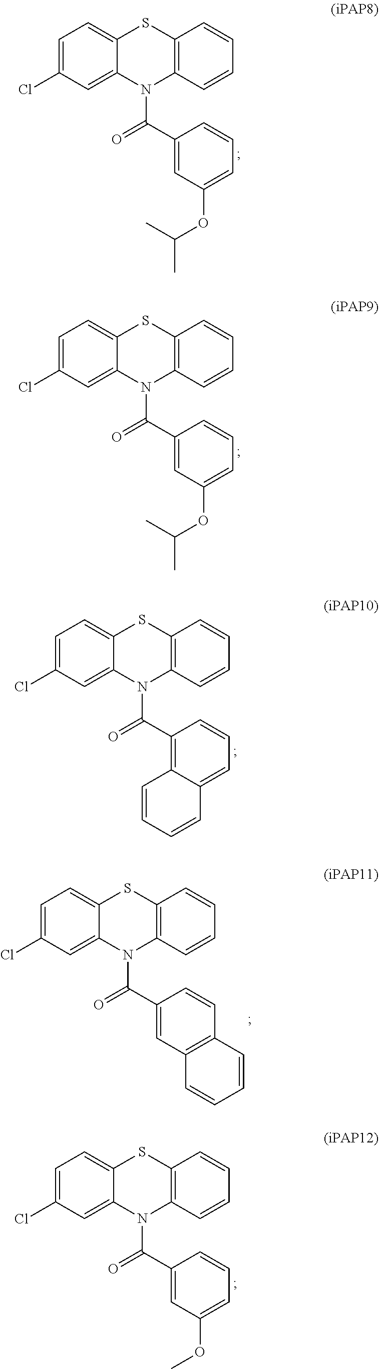 [SMILES: CC(C)Oc1cccc(C(=O)N2c3ccccc3Sc3ccc(Cl)cc32)c1 ; CC(C)Oc1cccc(C(=O)N2c3ccccc3Sc3ccc(Cl)cc32)c1 ; O=C(c1cccc2ccccc12)N1c2ccccc2Sc2ccc(Cl)cc21 ; O=C(c1ccc2ccccc2c1)N1c2ccccc2Sc2ccc(Cl)cc21 ; COc1cccc(C(=O)N2c3ccccc3Sc3ccc(Cl)cc32)c1]

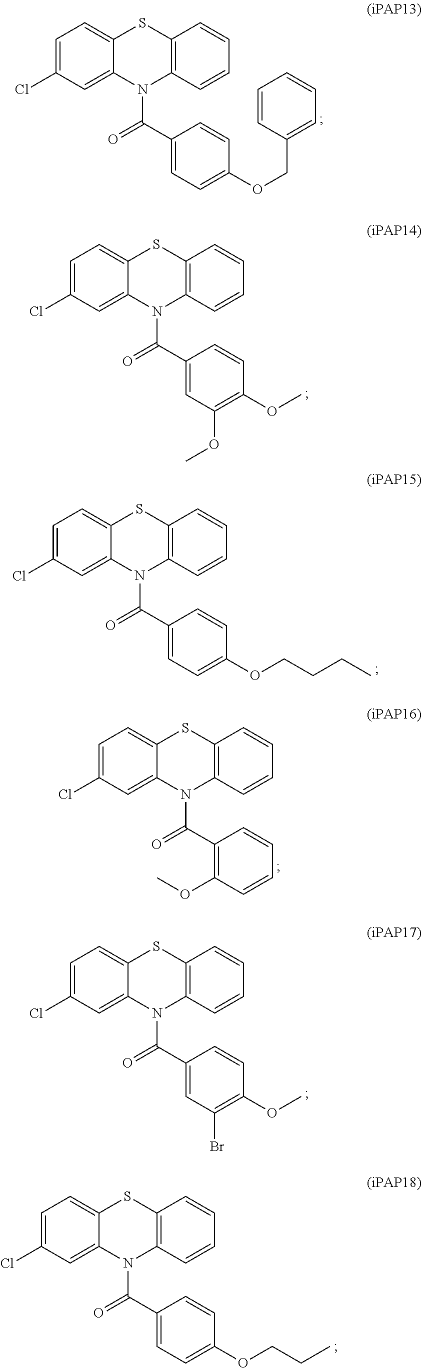 [SMILES: O=C(c1ccc(OCc2ccccc2)cc1)N1c2ccccc2Sc2ccc(Cl)cc21 ; COc1ccc(C(=O)N2c3ccccc3Sc3ccc(Cl)cc32)cc1OC ; CCCCOc1ccc(C(=O)N2c3ccccc3Sc3ccc(Cl)cc32)cc1 ; COc1ccccc1C(=O)N1c2ccccc2Sc2ccc(Cl)cc21 ; COc1ccc(C(=O)N2c3ccccc3Sc3ccc(Cl)cc32)cc1Br ; CCCOc1ccc(C(=O)N2c3ccccc3Sc3ccc(Cl)cc32)cc1]

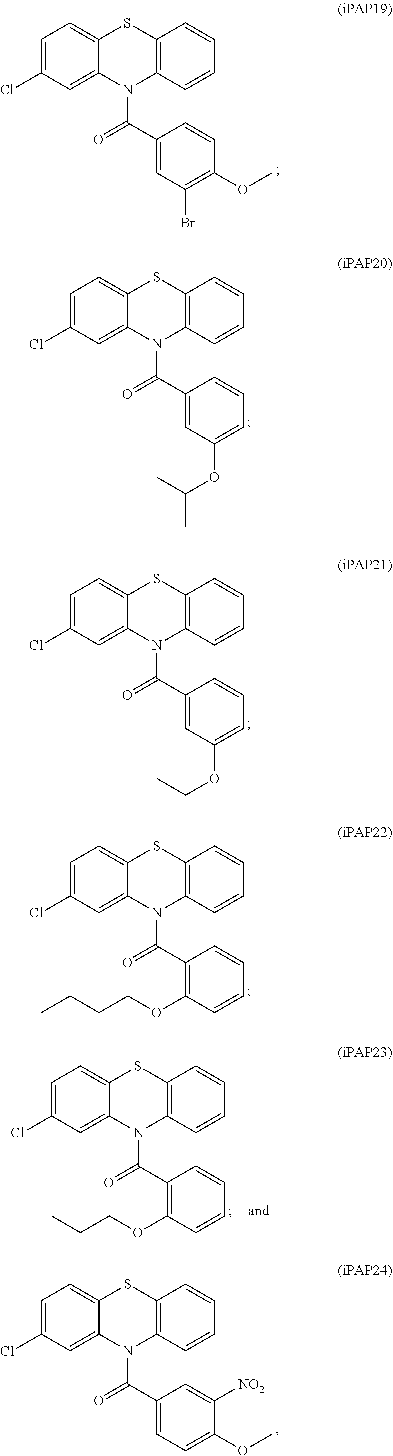 [SMILES: COc1ccc(C(=O)N2c3ccccc3Sc3ccc(Cl)cc32)cc1Br ; CC(C)Oc1cccc(C(=O)N2c3ccccc3Sc3ccc(Cl)cc32)c1 ; CCOc1cccc(C(=O)N2c3ccccc3Sc3ccc(Cl)cc32)c1 ; CCCCOc1ccccc1C(=O)N1c2ccccc2Sc2ccc(Cl)cc21 ; CCCOc1ccccc1C(=O)N1c2ccccc2Sc2ccc(Cl)cc21 ; COc1ccc(C(=O)N2c3ccccc3Sc3ccc(Cl)cc32)cc1[N+](=O)[O-]]

or a pharmaceutically or a pharmaceutically acceptable salt thereof.

In various embodiments, the method entails treating a hematological cancer such as acute myeloid leukemia (AML), B-cell acute leukemia (B-ALL), and B-cell non-Hodgkin's lymphomas and plasma cell myeloma.

In various embodiments, the method entails treatment of a subject with T-cell acute lymphoblastic leukemia (T-ALL). In various embodiments, the method of treating a subject with T-ALL entails administering a therapeutically effective amount of iPAP1 or a pharmaceutically acceptable salt thereof.

Other aspects of the invention are directed to a method of treating a cancer by administering to a subject a therapeutically effective amount of a perphenazine (PPZ) analog identified by selecting for optimal PP2A activity and a lack of inhibition of the dopamine D2 receptor as described herein. This is the approach that was used to identify the compounds of formula I and II and could be used to identify other similar drugs that are active in killing cancer cells such as neuroblastoma, small cell lung carcinoma, lung adenocarcinoma, gastric carcinoma, glioblastoma, medulloblastoma, primitive neuroectodermal tumor, meningioma, esophageal carcinoma, endometrial carcinoma, melanoma, head and neck carcinoma, renal cell carcinoma and breast cancer but do not cause movement disorders due to inhibiting the dopamine D2 receptor, which is the dose limiting side effect of PPZ (FIG. 24).

Applicant has surprisingly and unexpectedly discovered that the PPZ analogs of formulas I and II activate PP2A, but show no measurable inhibitory activity to DRD2. Thus, methods of the present invention may be effective in treatment of cancers that are susceptible to pharmacologically activated PP2A (e.g., T-ALL, T-cell non-Hodgkin lymphoma, acute myeloid leukemia (AML), chronic eosinophilic leukemia, chronic myeloid leukemia, B-cell acute lymphocytic leukemia (B-ALL), B-cell non-Hodgkin lymphoma, plasma cell myeloma, Hodgkin lymphoma, neuroblastoma, small cell lung carcinoma, lung adenocarcinoma and squamous cell carcinoma, gastric carcinoma, glioblastoma, primitive neuroectodermal tumor, meningioma, esophageal squamous cell carcinoma, endometrial carcinoma, medulloblastoma, melanoma, head and neck squamous cell carcinoma, pleural epithelioid mesothelioma, renal cell carcinoma, breast carcinoma, pancreatic ductal adenocarcinoma, ovarian carcinoma, osteosarcoma, and colon carcinoma as shown in FIG. 24), without substantially affecting activity of (e.g., inhibiting) DRD2, which may result in fewer deleterious side effects associated with the methods of treatment. Moreover, and without intending to be bound by any particular theory of operation, the PPZ analogs arrest cancer cells in prometaphase, which is the first phase of mitosis leading to cell division, through their ability to activate PP2A enzymatic activity.

A further of aspect of the invention is directed a method of treating thrombocytopenia by administering to a subject in need therof a therapeutically effective amount of a perphenazine (PPZ) analog identified by selecting for optimal PP2A activity and a lack of inhibition of the dopamine D2 receptor as described herein. In some embodiments, a therapeutically effective amount of a compound of formula (I) or (II), or a pharmaceutically acceptable salt, is administered to the subject. Without intending to be bound by theory, a consequence of the use of compounds of formula I and II to affect the traverse of normal megakaryocytes through prometaphase leads to increased endoreduplication of these cells which in turn causes them to produce more platelets. A related aspect concerns the use of the compounds disclosed herein in vitro to treat cultures of platelet-producing bone marrow stem cells or pluripotent stem (iPS) cells induced to form platelet producing cells. The addition of a disclosed compound may increase the output of platelets that can be harvested from these cultured human cells.

### BRIEF DESCRIPTION OF THE DRAWINGS

FIG. 1A-FIG. 1B are western blots showing that each of the PP2A A and C subunits were knocked out by CRISPR-Cas9 with unique gRNAs in KOPT-K1 cells. Two gRNAs with different target sequences were designed for each subunit. Control gRNAs target luciferase gene.

FIG. 2 is a western blot showing that each of the PP2A B subunits were knocked out by CRISPR-Cas9 with unique gRNAs in KOPT-K1 cells. Two gRNAs with different target sequences were designed for each subunit. Control gRNAs target luciferase gene.

FIG. 3A is a bar graph showing PPZ sensitivity in KOPT-K1 cells after PP2A subunit inactivation, and only each subunit of PP2A was knocked out by CRISPR-Cas9 with unique gRNAs. Two gRNAs with different target sequences were designed for each subunit (#1 and #2). Control gRNAs target luciferase gene. Data are presented as means±s.d. (n=3-5, biological replicates). ***P<0.001 by Student's t-test.

FIG. 3B is a bar graph showing small molecule activator of protein phosphatase (SMAP) sensitivity in KOPT-K1 cells after PP2A subunit inactivation, and only each subunit of PP2A was knocked out by CRISPR-Cas9 with unique gRNAs. Two gRNAs with different target sequences were designed for each subunit (#1 and #2). Control gRNAs target luciferase gene. Data are presented as means±s.d. (n=3-5, biological replicates). ***P<0.001 by Student's t-test.

FIG. 4A is a graph showing PPZ sensitivity in RPMI-8402 cells after CRISPR-Cas9 knockout of the key subunits identified in KOPT-K1 cells (see, FIG. 3A). As shown for KOPT-K1 cells in FIG. 3A, the PPP2R1A, PPP2CA and PPP2R5E subunits were required for the growth inhibitory activity of PPZ in RPMI-8402 cells. Data are presented as means±s.d. (n=3, biological replicates). *P<0.05, **P<0.01, ***P<0.001 by student's t-test.

FIG. 4B-FIG. 4D are bar graphs showing cell viability in KOPT-K1 Cells were treated with 0.5 μM iPAP1 (FIG. 4B) and 5 μM SMAP (FIG. 4C) for 72 hours (**P<0.01 and ***P<0.001 vs. control by Student's t-test; the data are means±SD of three biological replicates), and phosphatase activity of PP2A in control KOPT-K1 cells vs. KOPT-K1 cells with selective PP2A subunit inactivation (FIG. 4D) (KO indicates knockout. *P<0.05, **P<0.01 and ***P<0.001 vs. control by Student's t-test; the data are means±SD of three biological replicates).

FIG. 4E-FIG. 4H are bar graphs showing the sensitivity to iPAP1 (FIG. 4E), PPZ (FIG. 4F-FIG. 4G) and SMAP (FIG. 4H) by sublines of KOPT-K1 cells with individual PP2A subunit inactivation. *P<0.05, **P<0.01 and ***P<0.001 vs. Control by Student's t-test; the means±SD of 3-5 biological replicates were compared.

FIG. 5A is a graph showing relative expression levels of each of the subunits of PP2A for 16 different T-ALL cell lines. The expression level of each of the subunits was estimated from the signal intensities of probes for these RNAs using gene expression arrays (GEO: GSE90138).

FIG. 5B-FIG. 5D are a set of western blots (FIG. 5B-FIG. 5C) and a bar graph (FIG. 46D) of coimmunoprecipitation assays with purified human PP2A subunits produced in insect cells. FIG. 5B-FIG. 5C show the results of protein pull-down assays with anti-PPP2CA antibody and purified PP2A 1294 subunits of MYC-tagged PPP2R1A, HA-tagged PPP2CA and FLAG-tagged PPP2R5E (FIG. 5B) or FLAG-tagged PPP2R5C (FIG. 5C). FIG. 5D shows phosphatase activity of PP2A upon iPAP1 treatment, as assessed with purified PP2A subunits. *P<0.05 vs. Control by Student's t-test, comparing the means±SD of three biologic replicates.

FIG. 6 is a graph showing the phosphatase activity of PP2A in control KOPT-K1 cells and in cells with PP2A subunit inactivation. An increase in free phosphate after PPZ addition was observed only if each of the PPP2R1A, PPP2CA and PPP2R5E subunits was intact. Data are presented as means±s.d. (n=3, biological replicates). KO; knock out. **P<0.01, ***P<0.001 by student's t-test.

FIG. 7A-FIG. 7C are western blots showing the phosphorylation levels of endogenous P-ERK and P-AKT substrates of PP2A after PPZ treatment in KOPT-K1 cell populations with individual PP2A subunit knockouts. Knock out is abbreviated “KO”.

FIG. 8 is a western blot showing the expression levels of each of the subunits of PP2A n KOPT-K1 cells with or without treatment with PPZ. PPZ did not induce an altered expression level of any of the assayed PP2A subunits, indicating that it does not activate PP2A by altering subunit expression levels.

FIG. 9A is a western blot showing the results of a co-immunoprecipitation assay using an anti-PPP2CA antibody for immunoprecipitation in KOPT-K1 cells. Cells were treated with PPZ at 10 μM or control DMSO for 24 hours before lysed for protein extraction.

FIG. 9B is a western blot showing the results of a co-immunoprecipitation assay using an anti-PPP2CA antibody for immunoprecipitation in KOPT-K1 cells. Cells were treated with SMAP at 10 μM or control DMSO for 24 hours before they were lysed for protein extraction.

FIG. 10 is a western Blot showing the results of co-immunoprecipitation assays with anti-PPP2R5E antibody in KOPT-K1 cells. Cells were treated with PPZ at 10 μM or control DMSO for 24 hours at 4° C. before lysis for protein extraction. The binding of PPP2CA and PPP2R1A to PPP2R5E in the trimeric complex was detected with anti-PPP2R5E antibody only in the PPZ-treated lysates.

FIG. 11 is a western blot showing the results of a co-immunoprecipitation assay with an anti-PPP2R5E antibody in KOPT-K1 cells. Cells were first lysed for protein extraction, then protein lysates were incubated with PPZ at 10 μM (PPZ+) or DMSO control (PPZ−) for only one hour at room temperature before co-immunoprecipitation with the anti-PPP2R5E antibody.

FIG. 12 is a set of western blots showing identical results for co-immunoprecipitation assays with anti-PPP2R5E antibody in cells from SUPT-13, a different T-ALL cell line. As in KOPT-K1 cells, the binding of PPP2CA and PPP2R1A to PPP2R5E was detected only in the lysates treated with PPZ for 24 hours at 4° C.

FIG. 13 is set of western blots showing the protein expression of MYC-tagged PPP2R1A, FLAG-tagged PPP2R5E or PPP2R5C and HA-tagged PPP2CA mammalian expression vectors coding these proteins were transfected into HEK293T cells.

FIG. 14 is set of Coomassie-stained gels showing the purity of MYC-tagged PPP2R1A, FLAG-tagged PPP2R5E/PPP2R5C and HA-tagged PPP2CA proteins produced in insect cells.

FIG. 15 is set of western blots showing the results of protein pull-down assays by anti-PPP2CA antibody with purified subunits of PP2A produced in insect cells, MYC-tagged PPP2R1A, FLAG-tagged PPP2R5E and HA-tagged PPP2CA, after one hour treatment at room temperature of a mixture of 200 micrograms of each subunit in IP lysis buffer.

FIG. 16 is set of western blots showing the results of immunoprecipitation (IP) assays with the anti-PPP2CA antibody with purified subunits of PP2A produced in insect cells, MYC-tagged PPP2R1A, FLAG-tagged PPP2R5C and HA-tagged PPP2CA, after one hour treatment at room temperature of a mixture of 200 micrograms of each subunit in IP lysis buffer.

FIG. 17 is graph showing the results of PP2A phosphatase activity assay examined using purified PP2A subunits obtained from infected insect cells. Only subunit mixtures containing PPP2R5E along with PPP2R1A, and PPP2CA that were treated with PPZ have phosphatase activity. The data is presented as mean±s.d. (n=3, biological replicates). *P<0.05 by student's t-test.

FIG. 18A-FIG. 18F are graphs showing the cellular thermal shift assay (CETSA) curves for KOPT-K1 cell lysates with and without the addition of PPZ after incubation for 3 minutes for the times indicated. Data are presented as means±s.d. (n=3, biological replicates). *P<0.05, **P<0.01 by student's t-test.

FIG. 18G-FIG. 18L are graphs showing the CETSA curves for KOPT-K1 cell lysates with and without the addition of compound iPAP1 (for improved PP2A activator, perphenazine-derived) after incubation for 3 minutes for the times indicated. Data are presented as means±s.d. (n=3, biological replicates). *P<0.05, **P<0.01 by student's t-test.

FIG. 19A shows the western blot data from KOPT-K1 cell lysates treated or untreated with PPZ showing subunits of PP2A detected by subunit-specific antibodies at various temperatures after incubation with or without PPZ, which were quantified during CETSA by Image J software to produce the data plotted in FIG. 18A-FIG. 18F.

FIG. 19B shows the western blot data from KOPT-K1 cell lysates treated or untreated with iPAP1 showing subunits of PP2A detected by subunit-specific antibodies at various temperatures after incubation with or without iPAP1, which were quantified during CETSA by Image J software to produce the data plotted in FIG. 18G-FIG. 18L.

FIG. 19C-FIG. 19D are graphs showing the cellular thermal shift assay (CETSA) curves for KOPT-K1 cell lysates with and without the addition of PPZ after incubation for 3 minutes for the times indicated. Data are presented as means±s.d. (n=3, biological replicates).

FIG. 19E shows the quantitation of levels α and β tubulins detected by specific antibodies using western blotting quantified during CETSA by Image J software of KOPT-K1 cell lysates treated or untreated with PPZ at various temperatures for 3 minutes.

FIG. 19F-FIG. 19G are graphs showing the cellular thermal shift assay (CETSA) curves for KOPT-K1 cell lysates with and without the addition of iPAP1 after incubation for 3 minutes for the times indicated. Data are presented as means±s.d. (n=3, biological replicates).

FIG. 19H shows the quantitation of levels α and β tubulins detected by specific antibodies using western blotting. α and β tubulins were quantified during CETSA by Image J software of KOPT-K1 cell lysates treated or untreated with iPAP1 at various temperatures for 3 minutes.

FIG. 19I-FIG. 19J are graphs showing the results of a fluorescence-based tubulin polymerization assay performed with PPZ (FIG. 19I) and iPAP1 (FIG. 19J) at the indicated concentrations. Paclitaxel at 3 μM and vincristine at 2.5 and 5 μM were simultaneously tested as controls.

FIG. 19K is an image showing cytospins of KOPT-K1 cells stained with Acetocarmine (a-c), AlexaFluor 647 (red) anti-α tubulin antibody (d-f and j-l), and DAPI (g-i and j-l) for chromatin, microtubules and DNA, respectively. The cells were treated for 24 hours before analysis with DMSO control, PPZ (10 μM) or iPAP1 (1 μM).

FIG. 19L is an image showing cytospins of KOPT-K1 cells stained with Acetocarmine, AlexaFluor 647 (red) anti-α tubulin antibody, and DAPI for chromatin, microtubules and DNA, respectively. The cells were treated for 24 hours before analysis with DMSO control, PPZ (20 μM) or iPAP1 (2 and 5 μM).

FIG. 19M is an image showing KOPT-K1 cells stained with Acetocarmine, AlexaFluor 647 (red) anti-α tubulin antibody, and DAPI for chromatin, microtubules and DNA, respectively. The cells were treated for 24 hours before analysis with DMSO controlor Vincristine (0.0001 and 0.001 μM) for 24 hours.

FIG. 20 is an image diagrammatically illustrating the unique bioactivities of perphenazine (PPZ) and its analog, iPAP1. Biochemical assays showed that iPAP1 potently activates phosphatase activity of protein phosphatase 2A (PP2A) and induces apoptosis in T-cell acute lymphoblastic leukemia (T-ALL) cells, but has lost the ability to bind and inhibit DRD2.

FIG. 21A-FIG. 21B are graphs showing the results of the PP2A phosphatase activity using the PP2A Immunoprecipitation Phosphatase Assay Kit (Merck Millipore®). The left panel (FIG. 21A) shows the results with PPZ and right panel (FIG. 21B) with iPAP1 added for one hour at room temperature at the indicated concentrations in DMSO to mixtures of 200 ng each of MYC-tagged PPP2R1A, FLAG-tagged PPP2R5C and HA-tagged PPP2CA. iPAP1 showed equivalent PP2A activation activity at ˜10 times lower concentrations compared to PPZ. *P<0.05 by student's t-test.

FIG. 22 is a graph showing the results of the dopamine receptor D2 inhibition with PPZ and iPAP1. While PPZ showed strong inhibitory activity of DRD2 activity at concentrations as low as 0.5 μM, iPAP1 showed no inhibitory activity of DRD2 signaling at concentrations up to 4 μM.

FIG. 23A is a diagram showing the relationships among three parameters for PPZ and 84 analogs thereof, including iPAP1. In this graph the axes represent i) IC₅₀ values obtained after treating cells from the T-ALL cell line KOPT-K1 for 72 hours, and ii) PP2A activation potency of each compound when added to KOPTK1 cell lysates, and iii) inhibitory concentration of DRD2 signaling examined in HEK293T cells.

FIG. 23B is a diagram showing the relationships among the key three parameters shown in FIG. 23B. The X and Y axes represent the antileukemic potency and PP2A activation capacity respectively. The percent inhibition of the dopamine receptor D2 examined in HEK293T cells is represented by the size of the spheres, where the larger spheres indicate the stronger inhibitory potential.

FIG. 24 is a graph that shows the results of a PRISM (Profiling Relative Inhibition Simultaneously in Mixtures) analysis of the cell viability relative to DMSO control after treatment for 5 days with 5 μM concentration of PPZ or iPAP1 against 274 cancer cell lines from 39 distinct types of human cancers.

FIG. 25A-FIG. 25D show the in vivo anti-tumor activities of PPZ and iPAP1 in a zebrafish T-ALL model. iPAP1 more actively killed tumor cells than PPZ in vivo (Panels B and C) without showing any inhibitory activities on DRD2, which entail a movement disorder with loss of the ability to swim right side up in the water column (Panel A). **P<0.01, ****P<0.0001.

FIG. 26A-FIG. 26B are tables that show dose-dependent neurological toxicity of PPZ and iPAP1 tested in C57BL/6 mice. During the one-week monitoring period after initial treatment, mice treated with PPZ at 5 mg/kg body weight/dose or more showed neurological toxicity, establishing the maximum tolerated dose as 2.5 mg/kg. Mice treated with iPAP1 did not show any neurological toxicity when administered up to 80 mg/kg body weight/dose per day for more than 30 days.

FIG. 27 is a graph that shows the anti-tumor activities of PPZ and iPAP1 in vivo in immunodeficient NSG (NOD/Scid/IL2Rγ^{null}) mice xenotransplanted with KOPT-K1 cells. Each of the drugs was administered daily at the indicated dosages by oral gavage. While treatment with PPZ at its maximum tolerability dose (2.5 mg/kg/day) did not show any survival advantage over the control, treatment with iPAP1 at 2.5 mg/kg/day significantly extended the overall survival period over control or PPZ treatment cohorts. Favorable effects on the overall survival were even more significant with high-dose iPAP1 treatment at 80 mg/kg/day.

FIG. 28 is a graph that shows dose-response curves of human T-ALL cell lines (KOPT-K1, SUPT-13 and RPMI-8402) treated with PPZ or iPAP1 at various concentrations for 72 hours. iPAP1 was 10 times more potent in cell killing than PPZ in these T-ALL cell lines. The IC₅₀ for iPAP1 is 200 to 400 nM for these cell lines.

FIG. 29 is bar graph that shows a comparison of IC₅₀ values for various PP2A activators, including iPAP1 and the second best compound from FIG. 23A, P-491313983 (iPAP5). iPAP1 is more potent in inducing cell death in cancer cells than perphenazine and the other three reported PP2A activators, forskolin, fingolimide and SMAP.

FIG. 30 is bar graph that shows a comparison of DRD2 activities after treatment with various PP2A activators, including iPAP1 and P-491313983. Among the PP2A activators tested, forskolin had a mild DRD2 inhibition activity (˜30%), but other compounds including iPAP1, P-5491313983 (iPAP5), fingolimod and SMAP did not show inhibitory activities on DRD2.

FIG. 31A-FIG. 31B are flow cytometric DNA histograms that show the cell cycle status of KOPT-K1 cells treated with DMSO as control, PPZ or iPAP1 for 24 hours. Relative DNA content of cells in each of the samples was determined by measuring PI (propidium iodide) staining using flow cytometry.

FIG. 31C is a flow cytometric DNA histogram that shows the cell cycle status of KOPT-K1 cells treated with DMSO as control or SMAP for 24 hours. Relative DNA content of cells in each of the samples was determined by measuring PI (propidium iodide) staining using flow cytometry.

FIG. 32 shows acetocarmine and immunofluorescence staining of KOPT-K1 cells treated with DMSO as control (A,D, G, and J), PPZ at 10 μM (B, E, H, and K) or iPAP1 (C, F, I, and L) at 1 μM for 24 hours. For immunofluorescence staining, Alexa 647 (red)-anti-α tubulin antibody and DAPI were used to stain microtubules and DNA respectively.

FIG. 33 is bar graph that shows the relative mRNA expression of genes whose inducible CRISPR-cas9 knockout causes cell cycle arrest in prometaphase yielding spindle monopolarity (PLKJ, PLK4, AURKA, KIF11, SASS6, RCC1, HAUS8, TPX2, PCNT, CENPJ and TUBG1 (McKinley et al., Dev. Cell 40:405-420 (2017)). KOPT-K1 cells were treated with DMSO as control, PPZ at 10 μM or iPAP1 at 1 μM for 6 hours.

FIG. 34 is scatter plot of phosphopeptides identified by phosphoproteomics analysis using KOPT-K1 cells treated with PPZ at 10 μM or iPAP1 at 1 μM for 3 hours. Fold changes of the counts of phosphopeptides in KOPT-K1 cells treated with PPZ and iPAP1 over control are shown in X and Y axis, respectively.

FIG. 35 is cellular DNA flow cytometry histogram that shows the cell cycle status of KOPT-K1 cells afterMYBL2 knockdown using gene specific shRNAs. Expression of shRNAs was induced by 3 μM doxycycline for 24 hours, and cellular DNA content of cells in each of the samples was measured by PI (propidium iodide) staining. MYBL2 siRNA knockdown induced significant G2/M phase arrest with increased cells with 4N cellular DNA content of KOPT-K1 cells.

FIG. 36 is an acetocarmine and immunofluorescence staining of KOPT-K1 cells after MYBL2 knockdown using gene specific shRNAs. Expression of the shRNAs was induced by adding 3 μM doxycycline to the medium for 48 hours. For immunofluorescence staining, Alexa 647 (red)-anti-α tubulin antibody and DAPI were used to stain microtubules and DNA respectively. Like PPZ and iPAP1 treatment, MYBL2 inactivation induced prometaphase arrest in the cell cycle with spindle and microtubule monopolarity.

FIG. 37 is bar graph that shows the relative mRNA expression levels of genes that are involved in spindle and microtubule monopolarity (PLK1, PLK4, AURKA, KIF11, SASS6, RCC1, HAUS8, TPX2, PCNT, CENPJ and TUBG1 (McKinley et al., Dev. Cell, 40:405-420 (2017)). MYBL2 was inactivated using gene specific doxycycline-inducible shRNAs. Induction of shRNAs for 24 hours with 3 μM doxycycline significantly down-regulated the expression levels of most of these genes.

FIG. 38 shows cell proliferation curves of KOPT-K1 cells with or without MYBL2 knockdown using shRNA. As shown previously, MYBL2 gene knockdown led to a significant reduction in cell growth rate.

FIG. 39 shows cell proliferation curves of KOPT-K1 cells with or without MYBL2 inactivation using shRNA. Rescue of cell growth effects of shRNA-mediated inactivation of MYBL2 was attempted with a series of non-phosphorylatable alanine mutants MYBL2 (S241A, T266A, S282A, S241A/T266A, S241A/S282A, T266A/S282A and S241A/T266A/S282A. o/e; overexpression).

FIG. 40 is a histogram that shows the cell cycle status of KOPT-K1 cells after inducible MYBL2 knockdown using gene specific shRNA, demonstrating arrest of the cells in G2/M phase of the cell cycle with 4N DNA content.

FIG. 41 shows acetocarmine and immunofluorescence staining of KOPT-K1 cells after MYBL2 knockdown using gene specific shRNAs. The rescue experiment included simultaneous overexpression of wild type (WT) MYBL2 or series of mutant MYBL2 (S241A, S241D or transcriptional activation domain deletion (TAD_del)). Expression of shRNAs and MYBL2 were induced by 3 μM doxycycline for 24 hours.

FIG. 42A-FIG. 42B are bar graphs showing the IC₅₀ values for PPZ and iPAP1 in KOPT-K1 cells with the phospho-mimic aspartic acid mutant forms of MYBL2. In shMYBL2 knockout cells, the overexpression of mutant MYBL2 harboring S241D (S241D, S241D/T266D, S241D/S282D and S241D/T266D/S282D) conferred resistance to PPZ (FIG. 42A) or iPAP1 (FIG. 42B) treatment in KOPT-K1 cells.

FIG. 43A-FIG. 43B are bar graphs showing the relative activities of promoters for two representative MYBL2 target genes, PLK1 and KIF11, which each cause cell cycle arrest in prometaphase yielding spindle monopolarity (McKinley et al., Dev. Cell, 40:405-420 (2017)). HEK293T cells were transiently transfected with a vector expressing luciferase under control of either the PLK1 promoter or the KIF11 promoter. The activities of the promoters were measured by detecting luminescence.

FIG. 44 is a bar graph showing peripheral blood platelet counts in C57BL/6J mice treated with either by DMSO or iPAP1 at 80 mg/kg/day intraperitoneally for seven consecutive days. iPAP1 treatment significantly increased the platelet counts in the blood (P=0.013 by two-sided student's t-test).

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Broadly described herein is method of treating a cancer, comprising administering to a subject in need thereof a therapeutically effective amount of a perphenazine (PPZ) analog which has a structure represented by formula I or II:

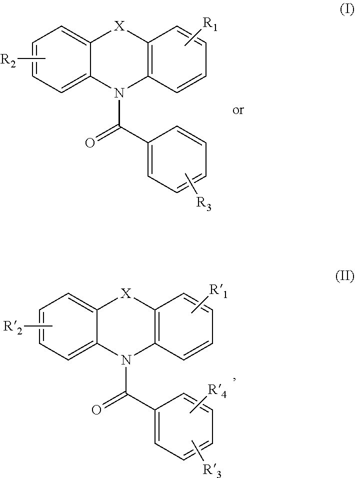

wherein X is O or S;
R₁ and R₂ are independently H, halo (e.g., Cl or F), NO₂ or CN;
R₃ is C1-C2 alkyl or methoxy;
R′₁ and R′₂ are independently H, halo, NO₂ or CN;
R′₃ and R′₄ are independently halo, NO₂, CN, C1-C2 alkyl, methoxy, ethoxy, propoxy, isopropoxy, butoxy or benzyloxy; or R′₃ and R′₄ together with the atoms to which they are bound form a 6-membered aryl or 6-membered heteroaryl group,
and the compound of formula I or II constitutively activates protein phosphatase 2A (PP2A) without blocking signaling through the dopamine D2 receptor or a pharmaceutically acceptable salt thereof.

In some embodiments, the compound of formula I or II has any one of the following structures:

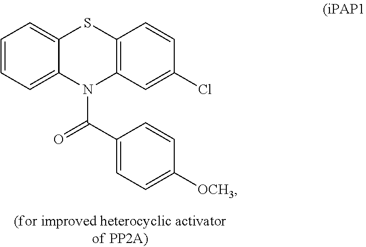 [SMILES: COc1ccc(C(=O)N2c3ccccc3Sc3ccc(Cl)cc32)cc1]

also known as iHAP1 (for improved heterocyclic activator of PP2A), Z56843374, P-889442 and 14B);

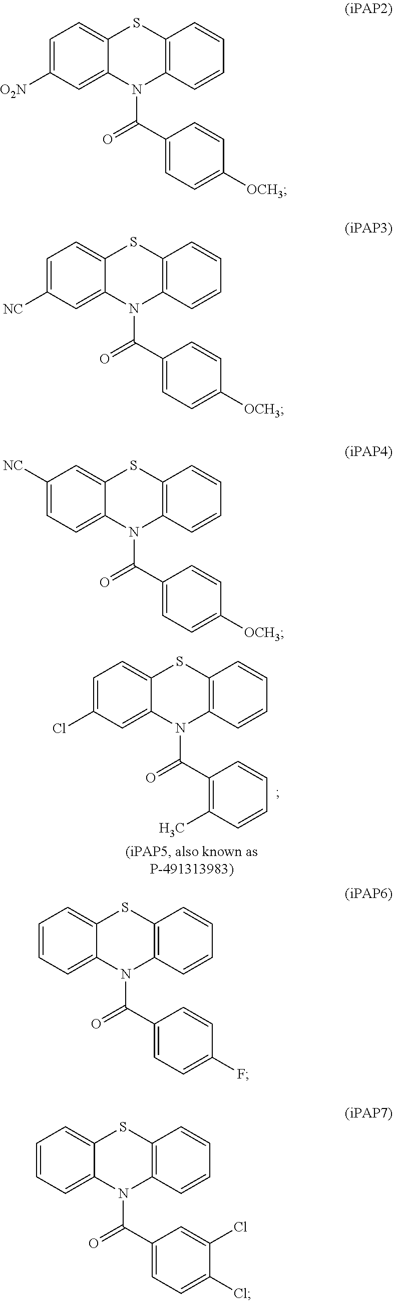

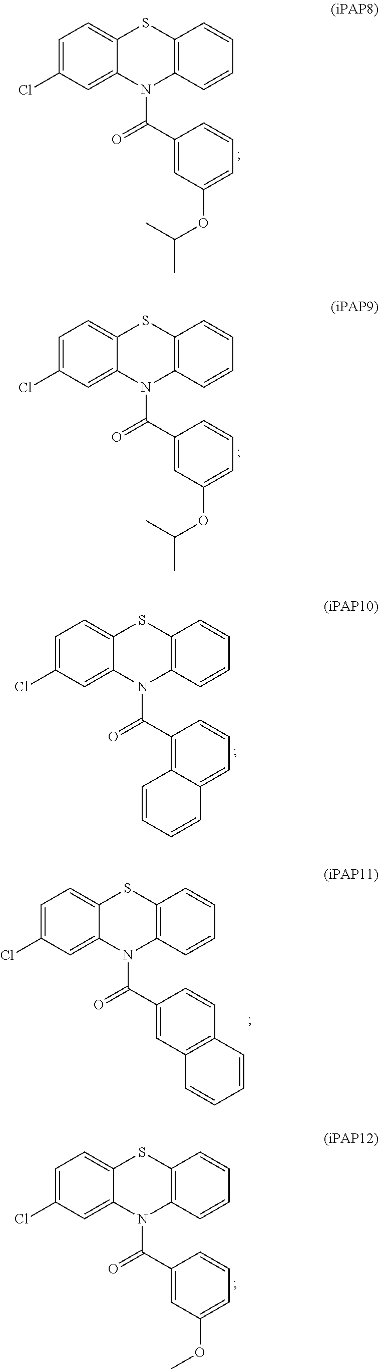 [SMILES: CC(C)Oc1cccc(C(=O)N2c3ccccc3Sc3ccc(Cl)cc32)c1 ; CC(C)Oc1cccc(C(=O)N2c3ccccc3Sc3ccc(Cl)cc32)c1 ; O=C(c1cccc2ccccc12)N1c2ccccc2Sc2ccc(Cl)cc21 ; O=C(c1ccc2ccccc2c1)N1c2ccccc2Sc2ccc(Cl)cc21 ; COc1cccc(C(=O)N2c3ccccc3Sc3ccc(Cl)cc32)c1]

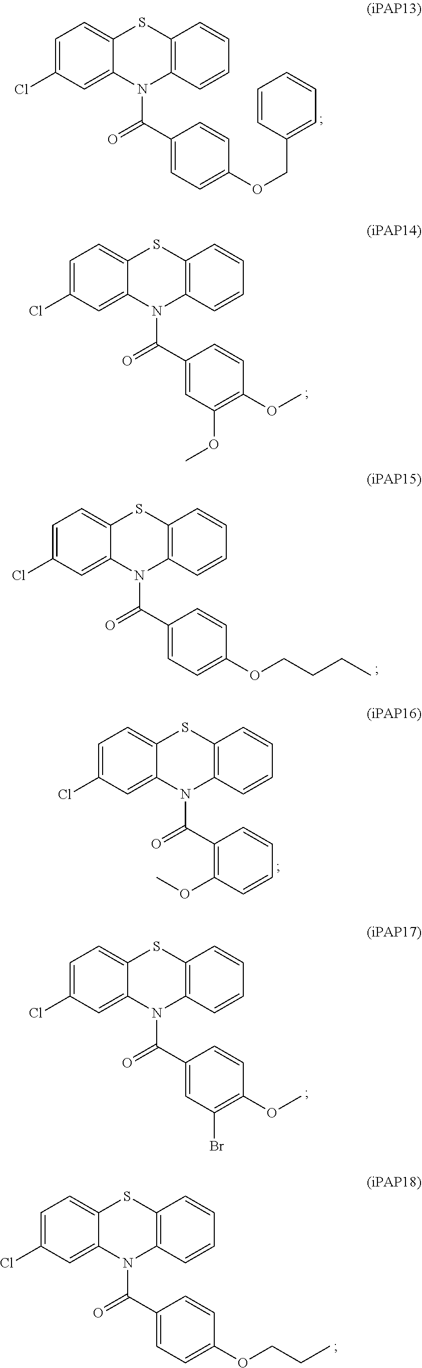 [SMILES: O=C(c1ccc(OCc2ccccc2)cc1)N1c2ccccc2Sc2ccc(Cl)cc21 ; COc1ccc(C(=O)N2c3ccccc3Sc3ccc(Cl)cc32)cc1OC ; CCCCOc1ccc(C(=O)N2c3ccccc3Sc3ccc(Cl)cc32)cc1 ; COc1ccccc1C(=O)N1c2ccccc2Sc2ccc(Cl)cc21 ; COc1ccc(C(=O)N2c3ccccc3Sc3ccc(Cl)cc32)cc1Br ; CCCOc1ccc(C(=O)N2c3ccccc3Sc3ccc(Cl)cc32)cc1]

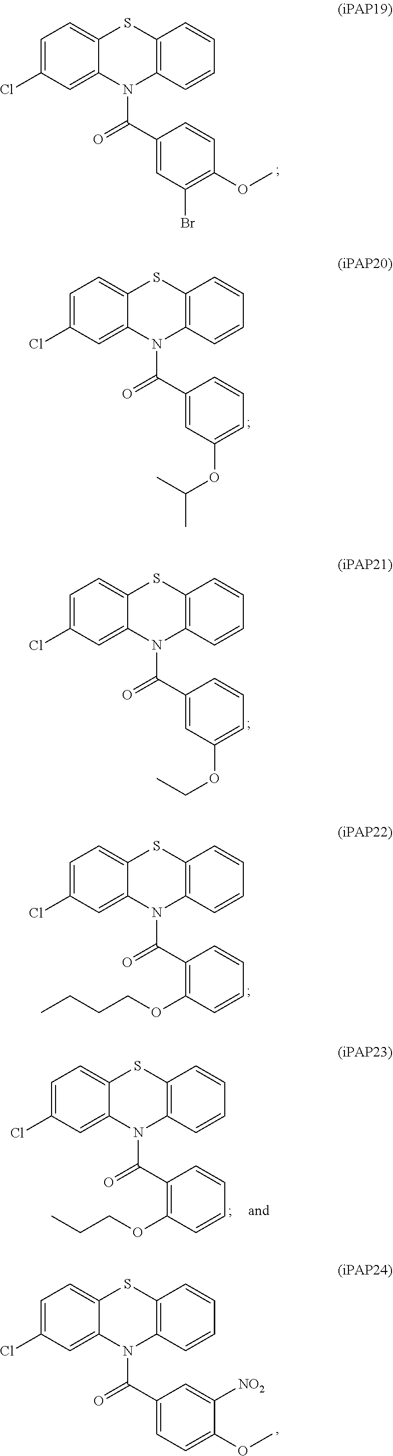 [SMILES: COc1ccc(C(=O)N2c3ccccc3Sc3ccc(Cl)cc32)cc1Br ; CC(C)Oc1cccc(C(=O)N2c3ccccc3Sc3ccc(Cl)cc32)c1 ; CCOc1cccc(C(=O)N2c3ccccc3Sc3ccc(Cl)cc32)c1 ; CCCCOc1ccccc1C(=O)N1c2ccccc2Sc2ccc(Cl)cc21 ; CCCOc1ccccc1C(=O)N1c2ccccc2Sc2ccc(Cl)cc21 ; COc1ccc(C(=O)N2c3ccccc3Sc3ccc(Cl)cc32)cc1[N+](=O)[O-]]

and pharmaceutically or a pharmaceutically acceptable salts thereof.

Compounds of formula I and (II) may be more potent activators of PP2A than PPZ, and yet lack the ability to bind and inhibit DRD2. Thus, the compounds described herein do not cause the DRD2-mediated central nervous system (CNS) side effects of PPZ, which, prior to the invention described herein, were known to be associated with treatment with PPZ.

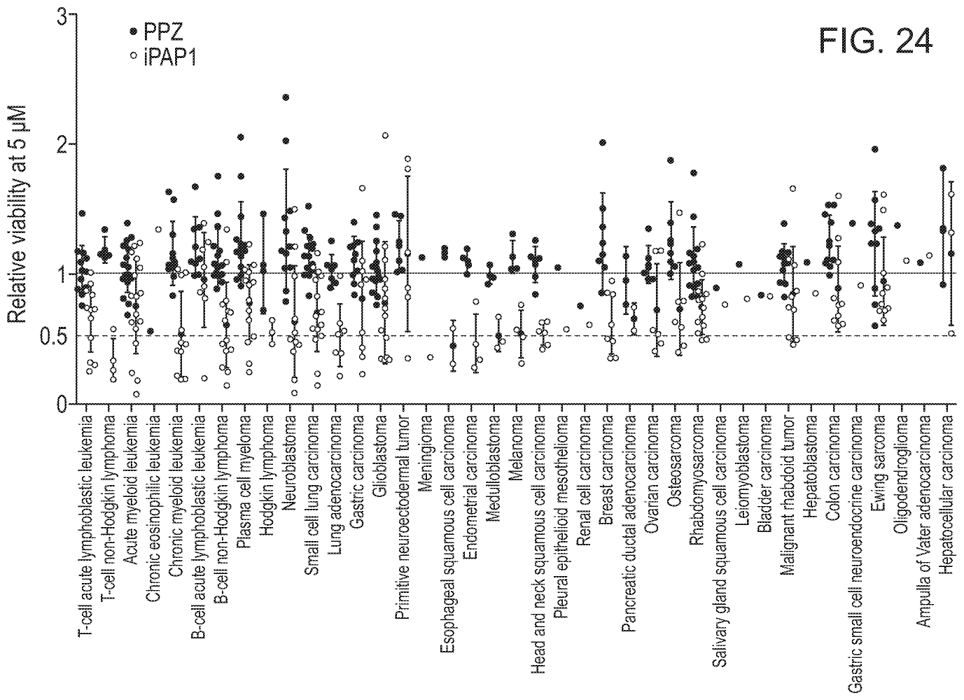

Another aspect of the present invention is directed to a method of treating a cancer by administering to a subject a therapeutically effective amount of a perphenazine (PPZ) analog identified by selecting for optimal PP2A activity and a lack of inhibition of the dopamine D2 receptor as described herein. This is the approach that was used to identify the compounds of formulas I and II and could be used to identify other similar drugs that are active in killing cancer cells such as neuroblastoma, small cell lung carcinoma, lung adenocarcinoma, gastric carcinoma, glioblastoma, medulloblastoma, primitive neuroectodermal tumor, meningioma, esophageal carcinoma, endometrial carcinoma, melanoma, head and neck carcinoma, renal cell carcinoma and breast cancer (see, FIG. 24) but do not cause movement disorders due to inhibiting the dopamine D2 receptor, which is the dose limiting side effect of PPZ.

In some embodiments, the cancer is T-cell acute lymphoblastic leukemia (T-ALL), T-cell non-Hodgkin lymphoma, acute myeloid leukemia (AML), chronic eosinophilic leukemia, chronic myeloid leukemia, B-cell acute lymphocytic leukemia (B-ALL), B-cell non-Hodgkin's lymphoma, plasma cell myeloma, Hodgkin lymphoma, neuroblastoma, small cell lung carcinoma, lung adenocarcinoma and squamous cell carcinoma, gastric carcinoma, glioblastoma, primitive neuroectodermal tumor, meningioma, esophageal squamous cell carcinoma, endometrial carcinoma, medulloblastoma, melanoma, head and neck squamous cell carcinoma, pleural epithelioid mesothelioma, renal cell carcinoma, breast carcinoma, pancreatic ductal adenocarcinoma, ovarian carcinoma, osteosarcoma, or colon carcinoma.

A further aspect of this invention is directed to the use of compounds of formula I and II to block cells in prometaphase with spindle and microtubule monopolarity by activating PP2A. PPZ analogs (e.g., iPAP1, iPAP2, iPAP3, iPAP4, and iPAP5) activate PP2A and this activation prevents tumor cells from completing prophase of the mitotic cycle, so that they die with 4N condensed chromosomes rather than completing mitosis. This block in the prophase occurs due to the ability of drugs like compounds of formula I and II to activate PP2A, and thus is likely due to interference with the activities of proteins that must be phosphorylated on serine/threonine to control the progression of cells through mitosis at the prometaphase step. Compounds of formulas I and II block the cell cycle in prometaphase producing a spindle and microtubule pattern called micropolarity by specifically removing phosphor-ser241 of MYBL2, which is required to activate the expression of genes required for cells to complete prometaphase.

A further of aspect of the invention is directed a method of treating thrombocytopenia by administering to a subject in need therof a therapeutically effective amount of a perphenazine (PPZ) analog identified by selecting for optimal PP2A activity and a lack of inhibition of the dopamine D2 receptor as described herein.

In some embodiments, a therapeutically effective amount of a compound of formula (I) or (II), or a pharmaceutically acceptable salt, is administered to the subject. A related aspect concerns the use of the compounds disclosed herein in vitro to treat cultures of platelet-producing bone marrow stem cells or pluripotent stem (iPS) cells induced to form platelet producing cells. The addition of a disclosed compound may increase the output of platelets that can be harvested from these cultured human cells.

Compounds of formulas I and II may be used in the form of a free acid or free base, or a pharmaceutically acceptable salt. As used herein, the term “pharmaceutically acceptable” in the context of a salt or ester refers to a salt or ester of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of formula I or II with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin. Representative examples of pharmaceutically acceptable esters include methyl, ethyl, isopropyl and tert-butyl esters.

In some embodiments, the compound of formula I or II is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

In addition, the compounds of formulas I and II embrace the use of N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds. The solvated forms of the conjugates presented herein are also considered to be disclosed herein.

### Pharmaceutical Compositions

For purposes of conducting the methods disclosed herein, compounds of formula I and II and their pharmaceutically acceptable salts may be formulated with or without a pharmaceutically acceptable carrier. For purposes of in vivo use, formulation with a carrier may be preferred. For purposes of in vitro use, the compound may be added directly to a culture medium without a carrier.

The term “pharmaceutically acceptable carrier,” as known in the art, refers to a pharmaceutically acceptable material, composition or vehicle, suitable for administering compounds of formulas I and II to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may further include one or more pharmaceutically acceptable excipients.

Broadly, compounds formulas I and II may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i. v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compounds of formulas I and II are formulated for oral or intravenous administration (e.g., systemic intravenous injection).

Accordingly, compounds of formulas I and II may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of formulas I and II are formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of formulas I and II may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

Compounds of formulas I and II may be formulated for buccal or sublingual administration, examples of which include tablets, lozenges and gels.

Compounds of formulas I and II may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of formulas I and II may be formulated for topical administration which as used herein, refers to administration intradermally by application of the formulation to the epidermis. These types of compositions are typically in the form of ointments, pastes, creams, lotions, gels, solutions and sprays.

Representative examples of carriers useful in formulating the bifunctional compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerine, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound of formula I or II with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of the compound of formula I or II or a pharmaceutically acceptable salt thereof effective in producing the desired therapeutic response in a particular patient suffering from thrombocytopenia or a cancer that is characterized by an anti-proliferative or apoptotic response to pharmacologically mediated upregulation of PP2A tumor suppressor activity. The term “therapeutically effective amount” includes the amount of the compound of formula I or II, or related PPZ analog lacking dopamine receptor D2 inhibitory activity, or a pharmaceutically acceptable salt thereof, which when administered, may induce a positive modification in the cancer (e.g., to constitutively activate tumor suppressor PP2A in cancer cells), or is sufficient to prevent development or progression of the cancer, or alleviate at least to some extent, one or more of the symptoms of the cancer in a subject.

The total daily dosage of the compounds of formulas I and II may be determined in accordance with standard medical practice, e.g., by an attending physician using sound medical judgment. Accordingly, the specific therapeutically effective dose for any particular subject may depend upon any one of a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the specific compound employed; and like factors well known in the medical arts (see, for example, Goodman and Gilman's, The Pharmacological Basis of Therapeutics, 10th Edition, A. Gilman, J. Hardman and L. Limbird, eds., McGraw-Hill Press (2001), at pages 155-173.

Compounds of formulas I and II may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day. These dosage amounts may also be applicable to the in vitro uses disclosed herein.

### Methods of Use

In some aspects, the present invention is directed to methods that include administering a therapeutically effective amount of a compound of formula I or II, or a pharmaceutically acceptable salt thereof, or a related PPZ analog lacking dopamine receptor D2 inhibitory activity, or a pharmaceutically acceptable salt thereof, to a subject in need thereof. The term “subject” (or “patient”) as used herein includes all members of the animal kingdom prone to or suffering from thrombocytopenia or a cancer (e.g., hematological cancer (e.g., T-ALL, T-cell non-Hodgkin lymphoma, acute myeloid leukemia (AML), chronic eosinophilic leukemia, chronic myeloid leukemia, B-cell acute lymphocytic leukemia (B-ALL), B-cell non-Hodgkin lymphoma, plasma cell myeloma, Hodgkin lymphoma), neuroblastoma, small cell lung carcinoma, lung adenocarcinoma and squamous cell carcinoma, gastric carcinoma, glioblastoma, primitive neuroectodermal tumor, meningioma, esophageal squamous cell carcinoma, endometrial carcinoma, medulloblastoma, melanoma, head and neck squamous cell carcinoma, pleural epithelioid mesothelioma, renal cell carcinoma, breast carcinoma, pancreatic ductal adenocarcinoma, ovarian carcinoma, osteosarcoma, and colon carcinoma). In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment may be “suffering from or suspected of suffering from” thrombocytopenia or a cancer that exhibits an antiproliferative or apoptotic response to activated PP2A activity may have a sufficient number of risk factors or presents with a sufficient number or combination of signs or symptoms such that a medical professional could diagnose or suspect that the subject was suffering from these types of cancers. Thus, subjects suffering from, and suspected of suffering from these types of cancers are not necessarily two distinct groups.

Compounds of formulas I and II or a related PPZ analog lacking dopamine receptor D2 inhibitory activity, or a pharmaceutically acceptable salt thereof, may be effective in the treatment of thrombocytopenia. In some embodiments, the method comprises treating cultures of platelet producing bone marrow stem cells or induced pluripotent stem (iPS) cells induced to form platelet producing cells as a means to increase the output of platelets with a compound of formula I or II or a related PPZ analog lacking dopamine receptor D2 inhibitory activity, or a pharmaceutically acceptable salt thereof, harvesting the cultured cells, and administering a therapeutically effective number of the cells to a subject in need thereof.

Compounds of formulas I and II and related PPZ analogs lacking dopamine receptor D2 inhibitory activity, and their pharmaceutically acceptable salts may be effective in the treatment of carcinomas (solid tumors including both primary and metastatic tumors), sarcomas, melanomas, neuroblastomas, and hematological cancers (cancers affecting blood including lymphocytes, bone marrow and/or lymph nodes) such as leukemia, lymphoma and multiple myeloma. Adult tumors/cancers and pediatric tumors/cancers are included (e.g., FIG. 24). The cancers may be vascularized, or not yet substantially vascularized, or non-vascularized tumors.

Representative examples of cancers include adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodeimal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma, Ann Arbor stage III and stage IV childhood Non-Hodgkin's lymphoma, ROS1-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

Other representative examples of cancers that may be amenable to treatment with the inventive methods include KRAS-driven cancers. KRAS-driven cancers include 90% of pancreatic cancers and 50% of colorectal and thyroid carcinomas, 30% non-small cell lung cancers (NSCLC), and 25% of ovarian cancers (Narvaez et al., Proc. Natl. Acad. Sci. 110(10):3937-42 (2013)).

In some embodiments, the methods are directed to treatment of a sarcoma. Sarcomas that may be treatable with the compounds of formulas I and II include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), mesenchymous or mixed mesodermal tumor (mixed connective tissue types), and histiocytic sarcoma (immune cancer).

In some embodiments, the methods are directed to treatment of a cell proliferative disease or disorder of the hematologic system. As used herein, “cell proliferative diseases or disorders of the hematologic system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic disease or disorder e.g., cancers, may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

In some embodiments, cell proliferative diseases or disorders of the hematological system include B-cell acute leukemia (B-ALL) and T-cell acute lymphoblastic leukemia (T-ALL).

In some embodiments, the methods are directed to treatment of a cell proliferative disease or disorder of the colon. As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polypopsis, Gardner's syndrome, Peutz-Jeghers syndrome, Turcot' s syndrome and juvenile polyposis. Cell proliferative disorders of the colon may also be characterized by hyperplasia, metaplasia, or dysplasia of the colon.

In some embodiments, the methods are directed to treatment of a cell proliferative disease or disorder of the pancreas. As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell types).

In some embodiments, the methods are directed to treatment of a cell proliferative disease or disorder of the prostate. As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

In some embodiments, the methods are directed to treatment of a cell proliferative disease or disorder of the skin. As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

In some embodiments, methods are directed to treatment of a cell proliferative disease or disorder of the ovary. As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

In some embodiments, methods are directed to treatment of a cell proliferative disease or disorder of the breast. As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

In some embodiments, the methods are directed to treatment of a cell proliferative disorder affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types).

In some embodiments, the methods are directed to treatment of non-metastatic or metastatic lung cancer (e.g., NSCLC, ALK-positive NSCLC, NSCLC harboring ROS1 rearrangement, lung adenocarcinoma, and squamous cell lung carcinoma).

The compound of formula I or a pharmaceutically acceptable salt thereof may be administered to a cancer patient, e.g., a T-ALL patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which were unsuccessful or partially successful but who became intolerant to the particular treatment. Therapy may also be given as an adjuvant treatment, i.e., to prevent reoccurrence of cancer in patients with no currently detectable disease or after surgical removal of a tumor. Thus, in some embodiments, the compound of formula I or II or a related PPZ analog lacking dopamine receptor D2 inhibitory activity, or a pharmaceutically acceptable salt thereof may be administered to a patient who has received another therapy, such as chemotherapy, radioimmunotherapy, surgical therapy, immunotherapy, radiation therapy, targeted therapy or any combination thereof.

The methods of the present invention may entail administration of compounds of the invention or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days). In other embodiments, the compound may be dosed twice a day (BID) over the course of two and a half days (for a total of 5 doses) or once a day (QD) over the course of two days (for a total of 2 doses). In other embodiments, the compound may be dosed once a day (QD) over the course of five days.

### Combination Therapy

The methods of the present invention may further include use of a compound of formula (I) or (II) or a related PPZ analog lacking dopamine receptor D2 inhibitory activity, or a pharmaceutically acceptable salt thereof, in combination with at least one other active anti-cancer agent, e.g., anti-TALL agent or regimen. The term “in combination” in this context means that the agents are co-administered, which includes substantially contemporaneous administration, by the same or separate dosage forms, or sequentially, e.g., as part of the same treatment regimen or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, a compound of the invention and the additional anti-cancer chemotherapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more anti-cancer therapeutics may be administered within the same patient visit.

In some embodiments, a compound of formula I or II and the additional agent or therapeutic (e.g., an anti-cancer therapeutic) are cyclically administered. Cyclic therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In some embodiments, the treatment regimen may include administration of a compound of formula I or II or a related PPZ analog lacking dopamine receptor D2 inhibitory activity, or a pharmaceutically acceptable salt thereof in combination with one or more additional anti-cancer therapeutics. The dosage of the additional anti-cancer therapeutic may be the same or even lower than known or recommended doses. See, Hardman et al., eds., Goodman & Gilman's The Pharmacological Basis Of Basis Of Therapeutics, 10th ed., McGraw-Hill, New York, 2001; Physician's Desk Reference, 60th ed., 2006. Anti-cancer agents that may be used in combination with the compound of formula I are known in the art. See, e.g., U.S. Pat. No. 9,101,622 (Section 5.2 thereof), U.S. Pat. No. 9,345,705 B2 (Columns 12-18 thereof), Litzo et al., Blood 126:833-841 (2015) and Raetz et al., Am. Soc. Hematol. Educ. Program. 1:580-588 (2015). Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bispecific antibodies) and CAR-T therapy.

In some embodiments, the methods of treating cancer (e.g., T-ALL) include administering the compound of formula I or II or a related PPZ analog lacking dopamine receptor D2 inhibitory activity, or a pharmaceutically acceptable salt thereof in combination with chemotherapy (e.g., nelarabine, methotrexate (MTX), and PEG-aspariginase), CNS radiation, or hematopoietic cell transplantation (HCT).

In some embodiments, the chemotherapeutic agent is daunarubicin or another anthracycline, vincristine, or VP16 or another epipodiphylotoxin.

In other embodiments, the methods of treating cancer include administering the compound of formula I or II or a related PPZ analog lacking dopamine receptor D2 inhibitory activity, or a pharmaceutically acceptable salt thereof in combination with prednisone or dexamethasone.

In some embodiments, the method of treating cancer (e.g., T-ALL) includes administering the compound of formula I or II or a related PPZ analog lacking dopamine receptor D2 inhibitory activity, or a pharmaceutically acceptable salt thereof in combination with an anti-Notch agent, e.g., a GSI. Exemplary GSIs include BMS-906024, BMS-986115, N—[N-(3,5-Difluorophenacetyl)-L-alanyl]-S-phenylglycine t-butyl ester (DAPT), LY90000, LY3039478, LY411575, MK-0752, PF-3084014, and R04929097. Other exemplary GSIs are described in Olsauskas-Kuprys et al., Onco Targets Ther. 6:943-955 (2013) and Ran et al., EMBO Mol. Med. 9(7):950-966 (2017).

In some embodiments, the anti-Notch agent is a Notch targeting monoclonal antibody (anti-Notch1, anti-Notch2, anti-anti-delta-like protein (DLL) 4) (e.g., OMP52M51, OMP59R5, and REGN421).

In some embodiments, the anti-Notch agent is Notch targeting soluble Notch proteins (e.g., SEL-10) or a Mastermind inhibiting peptide (e.g., SAHM1).

In some embodiments, the method of treating cancer (e.g., T-ALL) includes administering the compound of formula I or II or a related PPZ analog lacking dopamine receptor D2 inhibitory activity, or a pharmaceutically acceptable salt thereof in combination with an anti-phosphoinositide 3-kinase (PI3K)/AKT/mTOR agent, e.g., PI3K inhibitors. Exemplary PI3K inhibitors include BYL719, idelalisib, GSK2636771, BKM120, BAY80-6946, IPI145, TGR1202, AMG319, and SAR260301.

In some embodiments, the anti-PI3K/AKT/mTOR agent is a rapalog (mTOR inhibitor) (e.g., sirolimus, everolimus, temsirolimus, and ridaforolimus).

In some embodiments, the anti-PI3K/AKT/mTOR agent is a PIK3/mTOR inhibitor (e.g., BEZ235, GDC0980, VS5584, and SAR245409).

In some embodiments, the anti-PI3K/AKT/mTOR agent is an AKT inhibitor (e.g., MK2206 and GSK2110183).

In some embodiments, the anti-PI3K/AKT/mTOR agent is an mTORC1/2 inhibitor (e.g., OSI027, DS-3078a, and CC223).

In some embodiments, the method of treating cancer (e.g., T-ALL) includes administering the compound formula I or II or a related PPZ analog lacking dopamine receptor D2 inhibitory activity, or a pharmaceutically acceptable salt thereof in combination with an anti-JAK/STAT agent, e.g., Janus Kinase (JAK) 1/2 inhibitor. Exemplary JAK 1/2 inhibitors are ruxolitinib and momelotinib.

In some embodiments, the anti-JAK/STAT agent is a JAK 2 inhibitor (e.g., fedratinib, pacritinib, and BB594).

In some embodiments, the anti-JAK/STAT agent is a signal transducer and activator protein (STAT) inhibitor (e.g., C1889, pimozide, S31201, and STA21).

In some embodiments, the method of treating cancer (e.g., T-ALL) includes administering the compound of formula I or II or a related PPZ analog lacking dopamine receptor D2 inhibitory activity, or a pharmaceutically acceptable salt thereof in combination with an anti-mitogen-activated protein kinase (MAPK) agent, e.g., MEK inhibitor. Exemplary MEK inhibitors include trametinib, pimsertib, cobimetinib, and selumetinib.

In some embodiments, the anti-MAPK agent is a farnesyl transferase inhibitor (e.g., tipifarnib).

In some embodiments, the method of treating cancer (e.g., T-ALL) includes administering the compound of formula I or II or a related PPZ analog lacking dopamine receptor D2 inhibitory activity, or a pharmaceutically acceptable salt thereof in combination with an anti-cell-cycle machinery agent, e.g., a cyclin-dependent kinase (CDK) 4/6 inhibitor. Exemplary CDK 4/6 inhibitors include palbociclib, ribociclib, and abemaciclib.

In some embodiments, the anti-cell cycle machinery agent is a Pan-CDK inhibitor (e.g., flavopiridol, dinaciclib, and AT7519).

In some embodiments, the method of treating cancer (e.g., T-ALL) includes administering the compound of formula I or II or a related PPZ analog lacking dopamine receptor D2 inhibitory activity, or a pharmaceutically acceptable salt thereof in combination with an anti-proteasome, e.g., a proteasome inhibitor. Exemplary proteasome inhibitors include bortezomib, carfilzomib, and ixazomib.

In some embodiments, the anti-proteasome agent is a neddylation inhibitor (e.g., MLN49243).

In some embodiments, the anti-proteasome agent is a deubiquinating enzyme or an E3 ubiquitin ligase inhibitors.

In some embodiments, the method of treating cancer (e.g., T-ALL) includes administering the compound of formula I or II or a related PPZ analog lacking dopamine receptor D2 inhibitory activity, or a pharmaceutically acceptable salt thereof in combination with an anti-epigenetics agent, e.g., a histone deacetylase (HDAC) inhibitor. Exemplary HDAC inhibitors include vorinostat and romidepsin.

In some embodiments, the anti-epigenetics agent is a DNA methyltransferase inhibitor (e.g., 5-azacitidine and decitabine).

In some embodiments, the anti-epigenetics agent is an isocitrate dehydrogenase (IDH) 1/2 inhibitor (e.g., AGI6780, AGI5198, AG221).

In some embodiments, the anti-epigenetics is a bromodomain-containing protein 4 (BRD4) inhibitors (e.g., OTX015, and JQ1 and analogs thereof).

In some embodiments, the anti-epigenetics agent is a disruptor of telomeric silencing 1-like histone lysine methyltransferase (DOT1L) inhibitor (e.g., EPZ004777 and EPZ5676).

In some embodiments, the method of treating cancer (e.g., T-ALL) includes administering the compound of formula I or II or a related PPZ analog lacking dopamine receptor D2 inhibitory activity, or a pharmaceutically acceptable salt thereof in combination with immunotherapy (e.g., monoclonal antibodies (e.g., daratumomab, basiliximab, and al emtuzumab), bi-specific T-cell engagers, and chimeric antigen receptors (CARs)).

In some embodiments, the method of treating cancer (e.g., T-ALL) includes administering the compound of formula I or II or a related PPZ analog lacking dopamine receptor D2 inhibitory activity, or a pharmaceutically acceptable salt thereof in combination with hematopoietic cell transplantation.

In some embodiments, the method of treating cancer (e.g., T-ALL) includes administering the compound of formula I or II or a related PPZ analog lacking dopamine receptor D2 inhibitory activity, or a pharmaceutically acceptable salt thereof in combination with CNS radiotherapy.

In some embodiments, the method of treating thrombocytopenia includes administering the compound of formula I or II or a related PPZ analog lacking dopamine receptor D2 inhibitory activity, or a pharmaceutically acceptable salt thereof in combination with an anti-thrombocytopenia therapeutic (e.g., corticosteroids (e.g., predni sone), immunoglobulins, rituximab, eltrombopag, and romiplostim).

### Pharmaceutical Kits

The present compositions may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a compound of formula I or II or a pharmaceutical composition as disclosed herein. The kits or pharmaceutical systems of the invention may also include printed instructions for using the compounds and compositions.

These and other aspects of the present invention will be further appreciated upon consideration of the following Examples, which are intended to illustrate certain particular embodiments of the invention but are not intended to limit its scope, as defined by the claims.

### EXAMPLES

As background, multicellular eukaryotes including humans express two subtypes of A and C subunits (PPP2R1A and PPP2R1B for the A subunit; PPP2CA and PPP2CB for the C subunit), while at least 16 different genes encode the B subunits [B (PPP2R2A, PPP2R2B, PPP2R2C, PPP2R2D), B′ (PPP2R5A, PPP2R5B, PPP2R5C, PPP2R5D, PPP2R5E), B″ (PPP2R3A, PPP2R3B, PPP2R3C) and B′″ (STRN, STRN3, STRN4, PTPA)]. Human cells can theoretically produce 2×2×16=64 different heterotrimeric PP2A holoenzymes, which may account for a large fraction of the serine/threonine phosphatase activity in cells of various lineages. Unfortunately, however, the holoenzymes containing most of these subtypes of PP2A have not been studied systematically, and until our discovery it remained unclear which isoforms are most beneficial to target for activation to elicit tumor suppressor activities in cancer cells. In this aspect of the invention, the essential subunits of PP2A were determined for the anti-tumor activity of PPZ in T-ALL cells, and we establish that the role of PPZ is to nucleate the three subunits into a heterotrimeric holoenzyme with potent phosphatase activity.

### Example 1

### Identification of PP2A Subunits in KOPT-K1 Cells

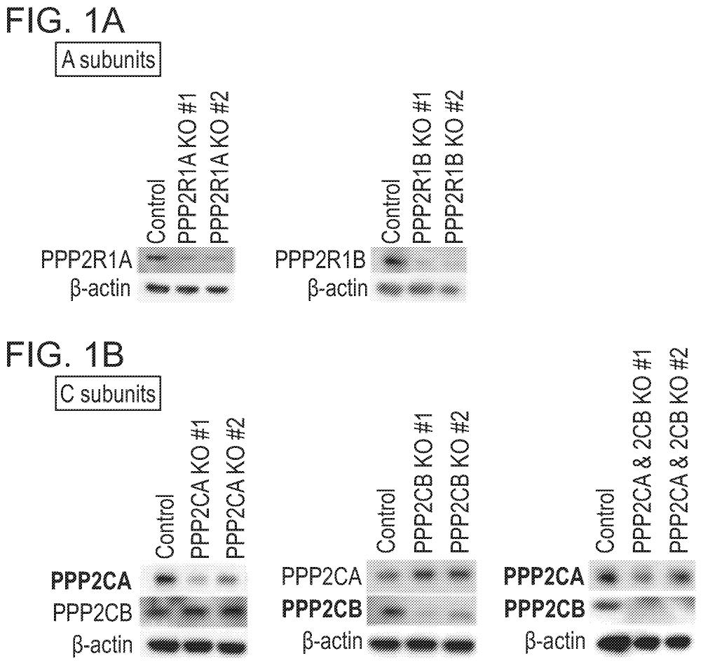

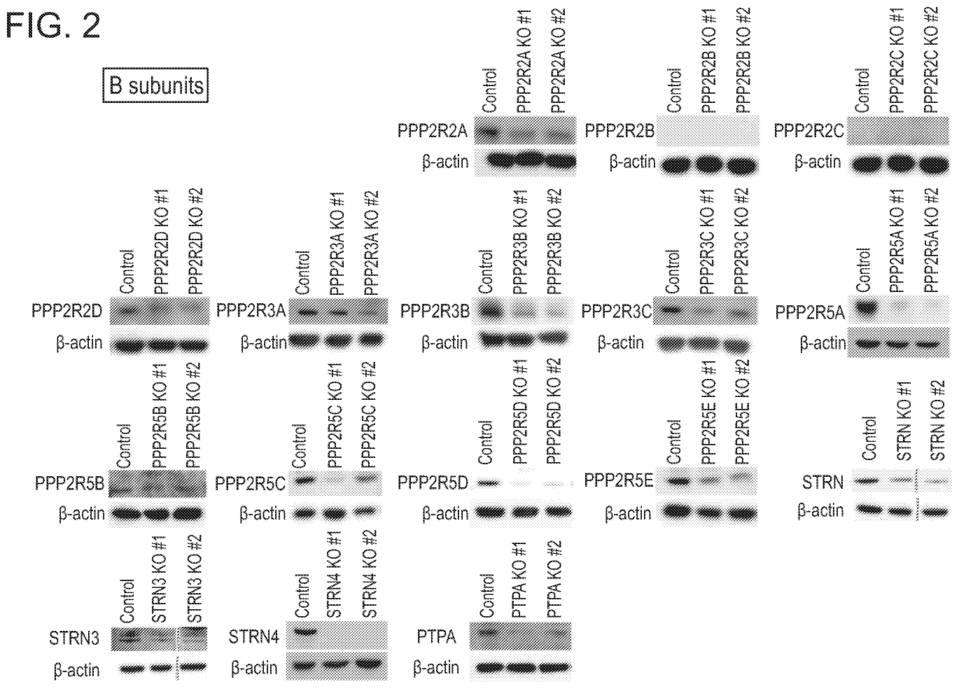

PP2A subunits A, B and C were knocked out by CRISPR-Cas9 with unique gRNAs in KOPT-K1 cells. Two gRNAs with different target sequences were designed for each subunit. Control gRNAs target luciferase gene. Knockout was validated by western blot (FIG. 1A, FIG. 1B, and FIG. 2). The basal expressions of PPP2R2B and PPP2R2C were undetectable.

### Example 2

### PPZ and Small Molecule Activator of Protein Phosphatase (SMAP) Sensitivity in KOPT-K1 and RPMI-8402 Cells

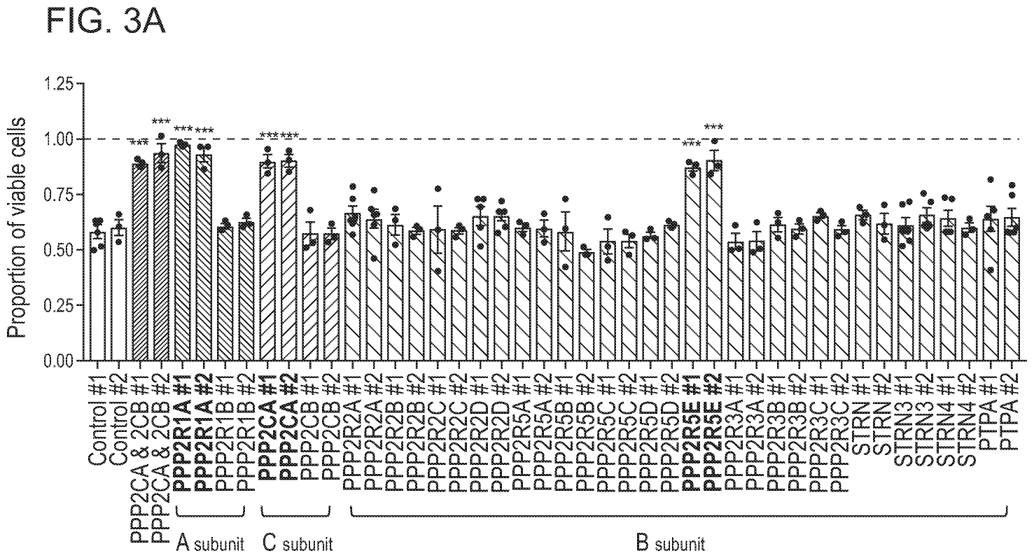

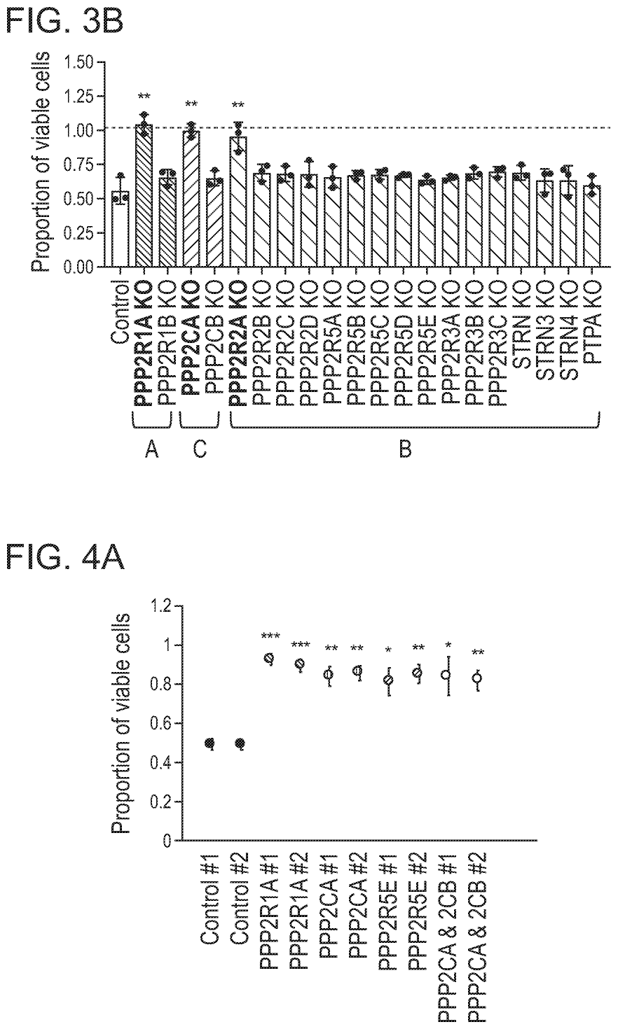

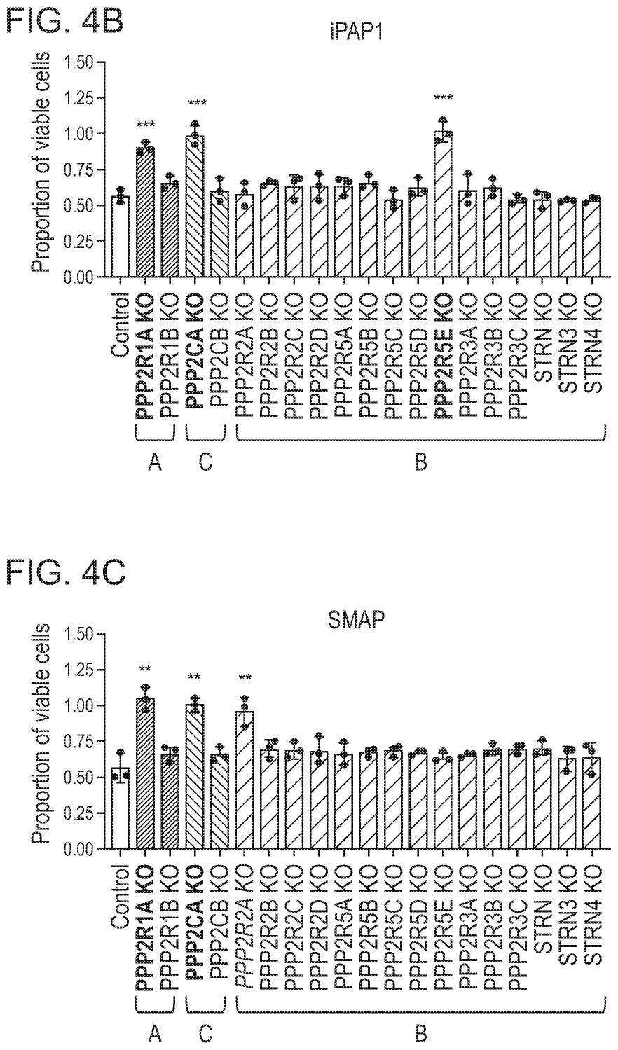

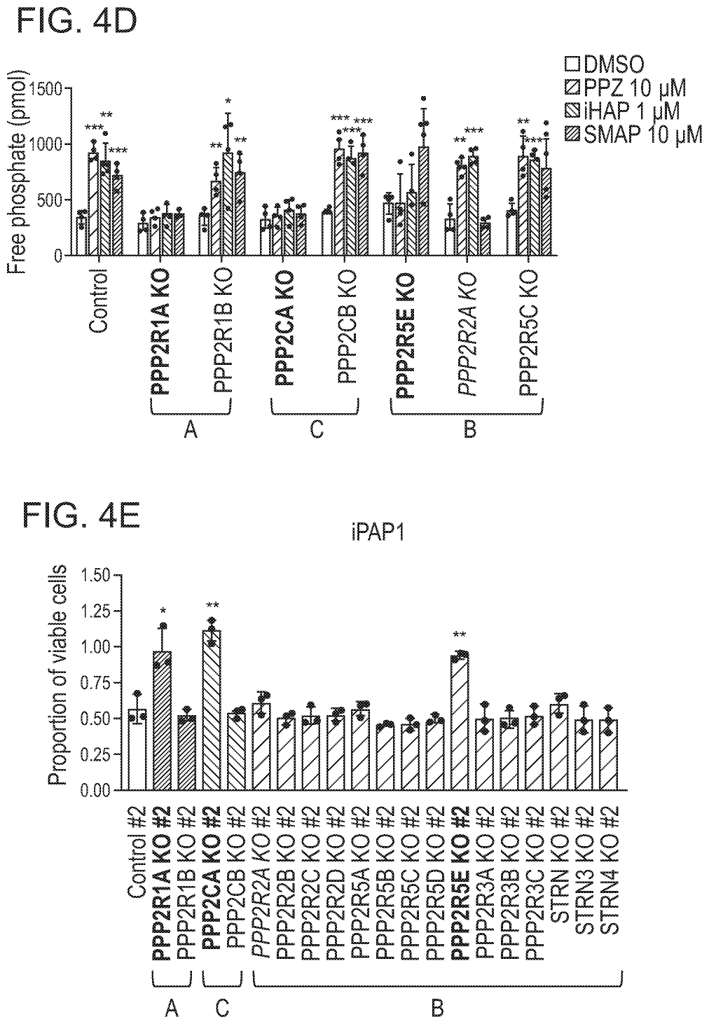

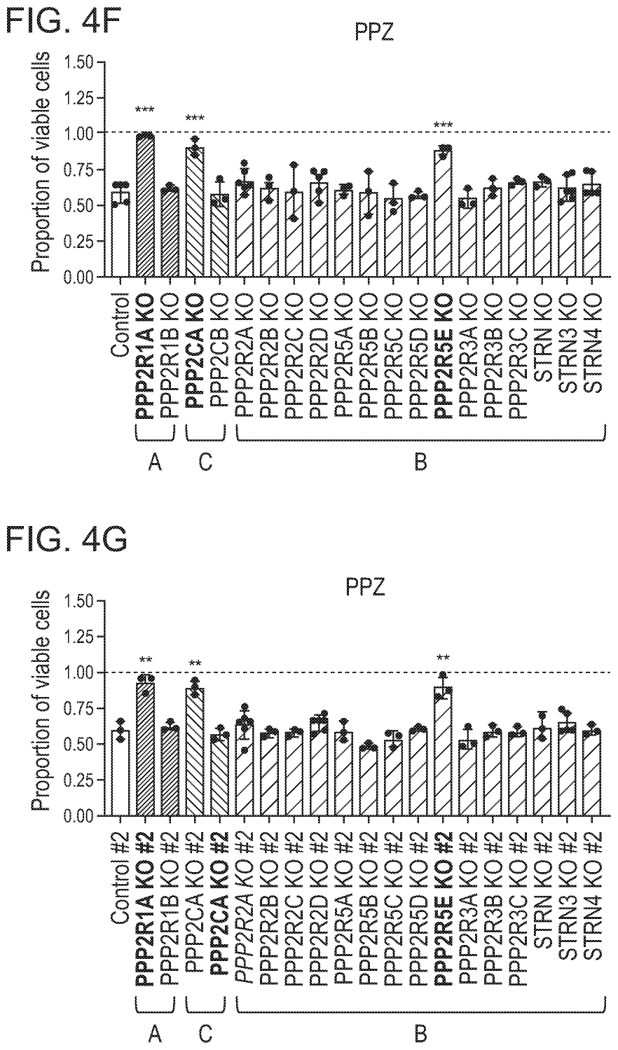

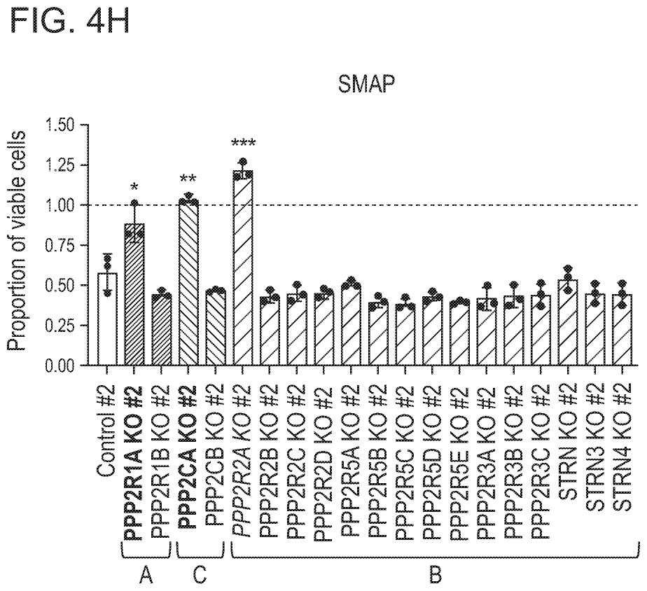

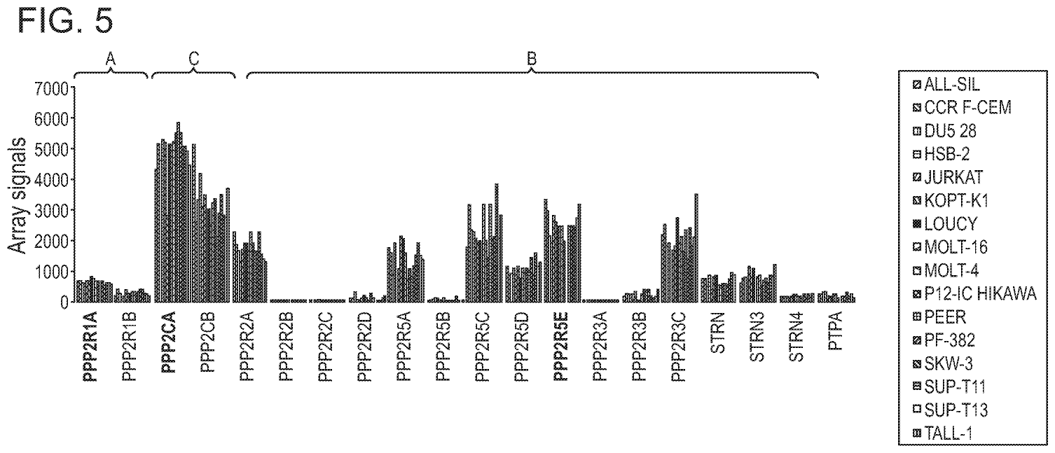

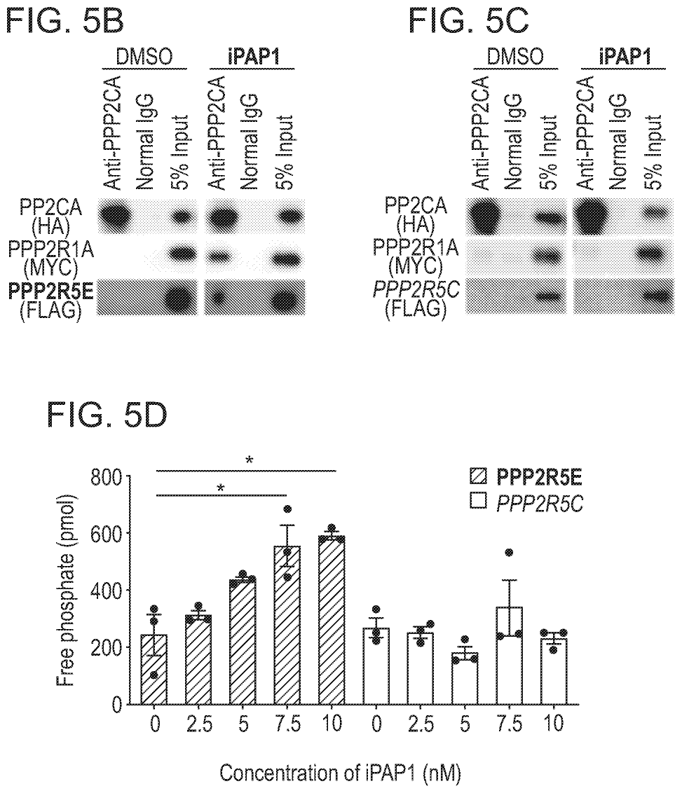

KOPT-K1 cells showed resistance to PPZ treatment only when the specific subunits PPP2R1A, PPP2CA or PPP2R5E were knocked out (FIG. 3A). RPMI-8402 cells, another T-ALL cell line, also showed resistance to PPZ treatment when these subunits were knocked out (FIG. 4A), indicating that these subunits are important for PPZ-mediated activation of PP2A and its anti-tumor activity in T-ALL cells in general. Consistent with our findings by CRISPR-Cas9 inactivation, these three subunits are were found among the most highly expressed PP2A subunits based on expression microarray analysis of a series of sixteen different T-ALL cell lines (FIG. 5). The expression level of each of the subunits was estimated from the signal intensities of probes for these RNAs using gene expression arrays (GEO: GSE90138).

PPZ sensitivity in KOPT-K1 cells was measured after PP2A subunit inactivation, and only each subunit of PP2A was knocked out by CRISPR-Cas9 with unique gRNAs. Only guide RNAs specific for the PPP2R1A, PPP2CA and PPP2R5E subunits caused the cells to lose sensitivity to PPZ, indicating that PPZ activates a trimeric holoenzyme consisting of these three subunits. Two gRNAs with different target sequences were designed for each subunit (#1 and #2). Control gRNAs target luciferase gene. Cells were treated with PPZ at 5 μM for 72 hours, then their viability was examined. Data are presented as means±s.d. (n=3-5, biological replicates). ***P<0.001 by Student's t-test (FIG. 3A).

SMAP sensitivity in KOPT-K1 cells was measured after PP2A subunit inactivation. Only each subunit of PP2A was knocked out by CRISPR-Cas9 with unique gRNAs. As observed with PPZ, only guide RNAs specific for the PPP2R1A, PPP2CA and PPP2R2A subunits caused the cells to lose sensitivity to SMAP, indicating that SMAP activates a trimeric holoenzyme consisting of these three subunits, and thus acts to activate a different trimeric holoenzyme, containing the “B subunit” PPP2R2A instead of PPP2R5E (see, e.g., Sangodkar et al., FEBS J. 283: 1004-1024 (2016); and Sangodkar et al., J. Clin. Invest. 127:2081-2090 (2017)). Two gRNAs with different target sequences were designed for each subunit (#1 and #2). Control gRNAs target luciferase gene. Data are presented as means±s.d. (n=3-5, biological replicates). ***P<0.001 by Student's t-test (FIG. 3B).

PPZ sensitivity was measured in RPMI-8402 cells after CRISPR-Cas9 knockout of the key subunits identified in KOPT-K1 cells as described above. As shown for KOPT-K1 cells in FIG. 3A, the PPP2R1A, PPP2CA and PPP2R5E subunits were required for the growth inhibitory activity of PPZ in RPMI-8402 cells. Cells were treated with PPZ at 5 μM for 72 hours, then their viability was examined. Data are presented as means±s.d. (n=3, biological replicates). *P<0.05, **P<0.01, ***P<0.001 by student's t-test (FIG. 4A).

To identify the subunits of PP2A that are essential for the antitumor activity of iPAP1, CRISPR-Cas9 was used to establish a series of sublines of KOPT-K1 T-ALL cells, each lacking one of the 19 specific PP2A subunits (FIG.1A-FIG. 2), and to examine their sensitivity to iPAP1-induced growth inhibition. Intriguingly, as shown in FIG. 4B and FIG. 4E, KOPT-K1 cells showed resistance to iPAP1 treatment only when the PPP2R1A, PPP2CA or PPP2R5E subunits were disrupted with specific guide RNAs. Using a similar approach, it was discovered that the antitumor activity of PPZ, like that of iPAP1, is mediated through the PPP2R1A, PPP2R5E and PPP2CA subunits in KOPT-K1 cells (FIG. 4F-FIG. 4G).

With this panel of PP2A subunit knockout cells in hand, we analyzed the PP2A subunits required for growth suppression by SMAPs, another class of allosteric activators of PP2A (Gutierrez et al., J. Clin. Invest. 124:644-655 (2014); Sangodkar et al., J. Clin. Invest. 127:2081-2090 (2017)). In the analysis of DT-061 (referred to as SMAP herein), tested cells were resistant to SMAP treatment only when the PPP2R1A, PPP2CA or PPP2R2A subunit was disrupted (FIG. 4C and FIG. 4H). Hence, SMAPs activate a PP2A holoenzyme with the PPP2R2A rather than the PPP2R5E regulatory subunit. Because the regulatory subunit of PP2A determines substrate specificity (Sangodkar et al., FEBS J. 283:1004-1024 (2016)), these findings indicate that the different PP2A complexes activated iPAP1/PPZ and SMAP likely target different signal transduction pathways. Both the PPP2R5E and PPP2R2A regulatory subunits of PP2A were among the most highly expressed in a series of 16 different T-ALL cell lines (FIG. 5A). Thus, the assembly of structurally different heterotrimeric PP2A complexes induced with either iPAP1 or SMAP cannot be attributed to imbalances in cellular expression levels of individual PP2A subunits, but likely stems from differences in the allosteric alterations of the A subunit protein resulting from the binding of these two classes of small molecules, which have different chemical structures.

As shown in FIG. 4D, the phosphatase activity of PP2A was increased in WT KOPT-K1 cells, up to twice its basal level, upon treatment with iPAP1 (1 μM), PPZ (10 μM) or SMAP (10 μM). With this assay, KOPT-K1 cells lacking functional PPP2R1A, PPP2CA or PPP2R5E, did not show increased phosphatase activity when treated with iPAP1, while in cells lacking other subunits, the iPAP1-induced phosphatase activity resembled that of WT control cells (FIG. 4D). Similar results were obtained for PPZ treatment of KOPT-K1 cells (FIG. 4D).

By contrast, SMAP treatment failed to induce phosphatase activity in KOPT-K1 cells when the PPP2R1A, PPP2CA or PPP2R2A (instead of PPP2R5E) subunit was disrupted (FIG. 4D).

Taken together, these results show that iPAP1/PPZ and SMAP assemble active PP2A phosphatases containing different regulatory subunits—PPP2R5E for iPAP1/PPZ and PPP2R2A for SMAP—and that both PP2A phosphatases can catalyze the release of free phosphate from the threonine phosphopeptide.

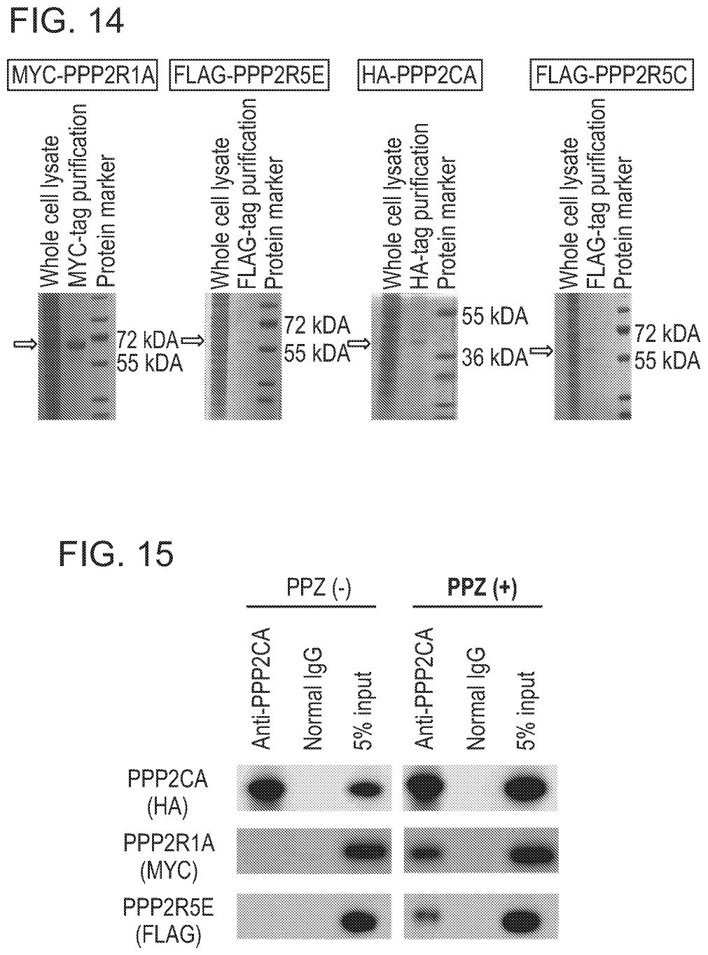

The biochemical activity of iPAP1 was further characterized by addressing whether it acts exclusively on the three identified subunits to mediate the assembly of PP2A, or whether other proteins expressed by T-ALL cells are also required. Using baculovirus vectors, each subunit of PP2A was first expressed with a C-terminal tag in Hi5 insect cells, and then purified the subunit proteins using columns with antibodies against the tags bound to agarose beads (see, Example 5, FIG. 13-FIG. 14). As in T-ALL cell lysates, PPP2R1A, PPP2CA and PPP2R5E were assembled into an active enzyme complex in the presence of iPAP1 (FIG. 5B), while substitution of the PPP2R5C for the PPP2R5E regulatory subunit abrogated the formation of a complex (FIG. 5C). Consistent with these findings, the phosphatase activity of PP2A was increased upon iPAP1 treatment if this holoenzyme was assembled from the affinity-purified PPP2R1A, PPP2CA and PPP2R5E subunits, but its activity was unchanged from background levels if PPP2R5C was substituted for PPP2R5E (FIG. 5B).

### Example 3

### Phosphatase Activity of PP2A in KOPT-K1 Cells upon PPZ Treatment

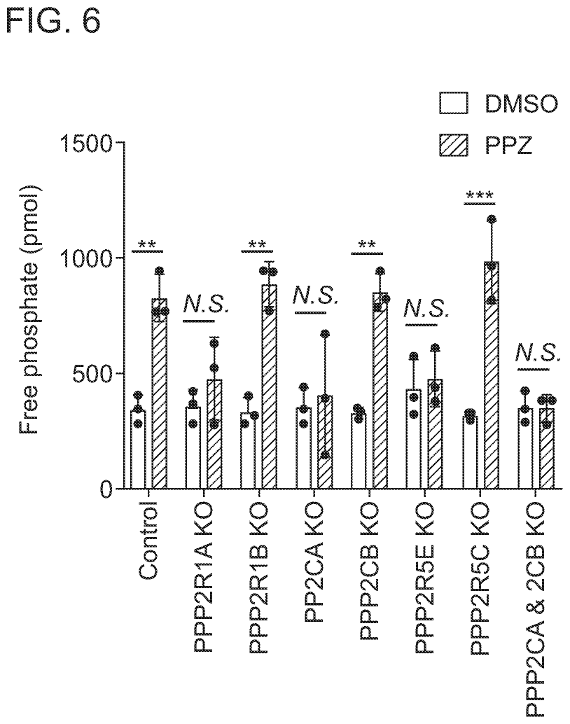

The phosphatase activity of PP2A was increased up to two-fold from its basal activity upon PPZ treatment. Using this assay, KOPT-K1 cells treated with PPZ but lacking PPP2R1A, PPP2CA or PPP2R5E did not show increased phosphatase activity, while cells lacking PPP2R1B, PPP2CB, or PPP2R5C resembled the control and showed increased phosphatase activity upon PPZ treatment (FIG. 6). Cells were treated with PPZ at 10 μM for three hours before the activity of PP2A was quantified. Data are presented as means±s.d. (n=3, biological replicates). KO; knock out. **P<0.01, ***P<0.001 by student's t-test.

### Example 4

### Phosphorylation of Endogenous P-ERK and P-AKT Substrates of PP2A After PPZ Treatment in KOPT-K1 Cells

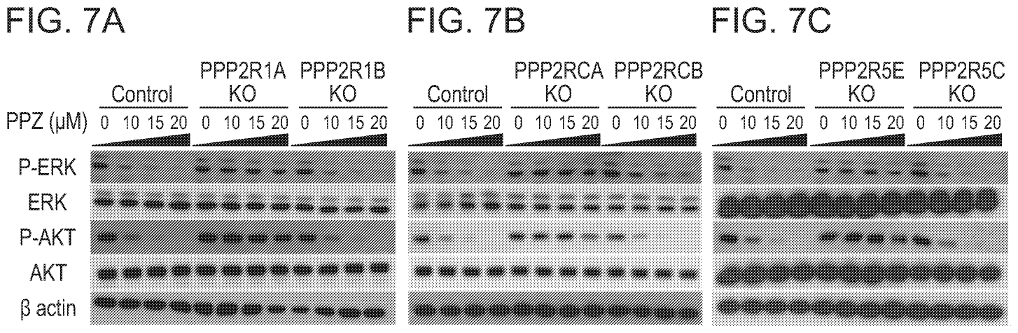

Levels of the PP2A endogenous substrates p-ERK and p-AKT in KOPT-K1 cells were examined because of the importance of the MAPK/ERK and PI3K/AKT/mTOR pathways in cancer cells. In control KOPT-K1 cells, phosphorylation of ERK and AKT were each significantly reduced upon PPZ treatment. By contrast, phosphorylation of these proteins was unchanged by PPZ treatment of KOPT-K1 cells with loss of PPP2R1A, PPP2CA or PPP2R5E, except for slight decreases in phosphorylation in some instances at the highest 20 μM concentration of PPZ, consistent with small amounts of residual protein expression in these CRISPR-cas9 treated cells (FIG. 7A-FIG. 7C). Cells were treated with PPZ at the indicated concentrations for three hours, then the phosphorylation status of ERK and AKT was measured by western blot. As controls, KOPT-K1 cells with CRISPR-Cas9-mediated inactivation of PPP2R1B, PPP2CB or PPP2R5C showed loss of P-ERK or P-AKT equivalent to control cells when treated with PPZ. These results indicate that PPP2R1A, PPP2CA and PPP2R5E are the three indispensable subunits of PP2A that mediate its phosphatase activity in T-ALL cells treated with PPZ.

### Example 5

### Mechanism of PPZ-Mediated Activation of PP2A in T-ALL Cells

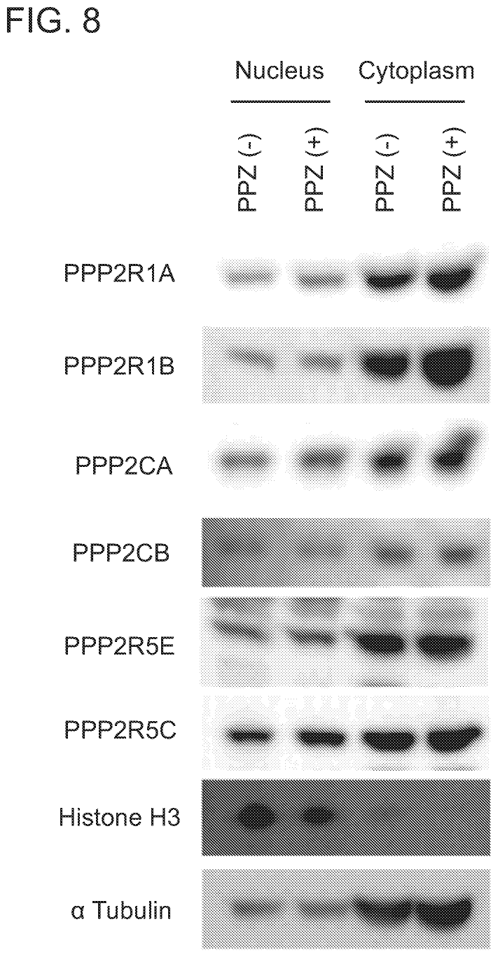

PP2A is a unique enzyme consisting of three subunits and it needs to be properly assembled into a holoenzyme before it mediates phosphatase activity. It is hypothesized that PPZ might activate the function of PP2A in T-ALL cells by facilitating the assembly of its subunits. To test this hypothesis, KOPT-K1 cell were treated with PPZ at 10 μM or equivalent amount of dimethyl sulfoxide (DMSO) for 24 hours, subsequently lysed for protein extraction. The protein lysates were then immunoprecipitated with anti-PPP2CA antibody or normal IgG as a control, and the precipitates were immunoblotted with antibodies specifically detecting each of the PP2A subunits. Each of the PP2A subunits—PPP2R1A, PPP2R1B, PPP2R5E, PPP2R5C, PPP2CA and PPP2CB—are endogenously expressed by KOPTK1 cells, and PPZ did not alter the expression levels of these subunits in the nucleus or the cytoplasm of KOPT-K1 cells during the course of the experiment (FIG. 8). PPZ did not induce an altered expression level of any of the assayed PP2A subunits, indicating that it does not activate PP2A by altering subunit expression levels. Nuclear and cytoplasmic fractions were separated using NE-PER™ Nuclear and Cytoplasmic Extraction Kit (Thermo Fisher Scientific), and each of the subunits of PP2A was analyzed by western blot. Histone H3 and α tubulin expression levels were used as loading controls for nuclear and cytoplasmic fractions, respectively.

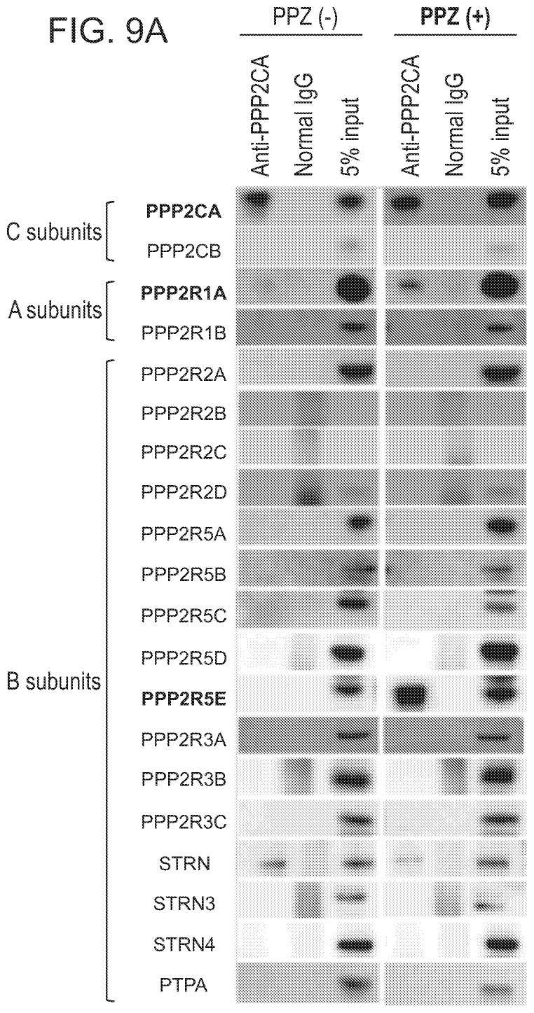

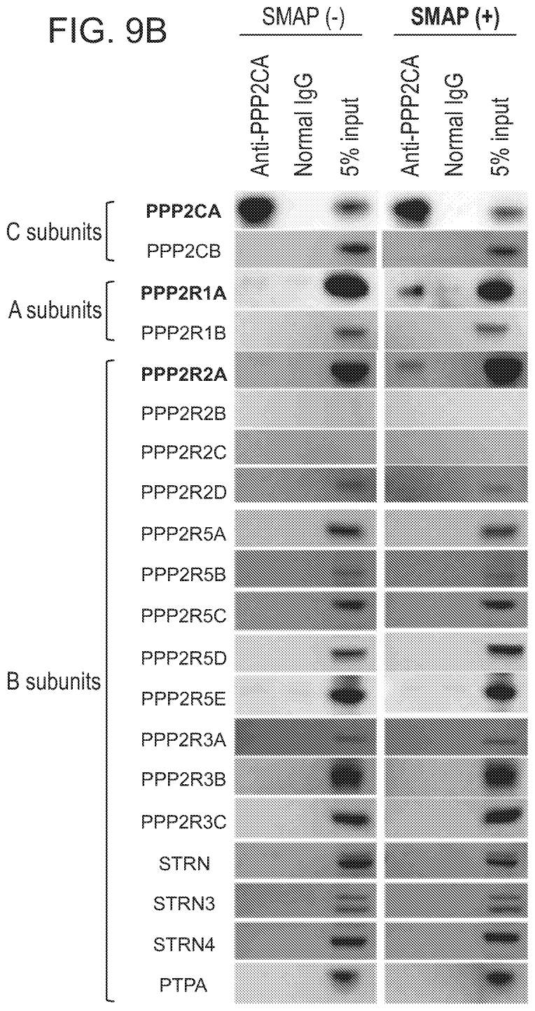

As shown in FIG. 9A, this immunoprecipitation (IP) pulldown assay showed that PPP2CA uniquely binds to PPP2R1A and PPP2R5E, but only in cells that had been treated with PPZ. Cells were treated with PPZ at 10 μM or control DMSO for 24 hours before lysed for protein extraction. Lysates were immunoprecipitated using an anti-PPP2CA antibody, and then blotted with antibodies specific for each PP2A subunit. Only PPP2R1A, PPP2R5E and PPP2CA were specifically immunoprecipitated after the addition of PPZ, indicating that the trimeric complex nucleated by PPZ contains PPP2R1A, PPP2R5E and PPP2CA.

The results of a co-immunoprecipitation assay using an anti-PPP2CA antibody for immunoprecipitation in KOPT-K1 cells treated with SMAP at 10 μM or control DMSO for 24 hours before they were lysed for protein extraction are illustrated in FIG. 9B. Lysates were immunoprecipitated using an anti-PPP2CA antibody, and then blotted with antibodies specific for each PP2A subunit. Only PPP2R1A and PPP2R2A were specifically immunoprecipitated after the addition of SMAP, indicating that the trimeric complex nucleated by SMAP contains PPP2CA, PPP2R1A and PPP2R2A. Thus, the PP2A complex that formed in response to SMAP is different than the PP2A complex that formed after the addition of PPZ.

FIG. 3B and FIG. 9B show the activation of a PP2A phosphatase containing the PPP2R2A “B subunit” by SMAP, while iPAP1, iPAP2, iPAP3, iPAP4 activate a PP2A phosphatase containing the PPP2R5E “B subunit”. Since the B subunit determines the specificity of the phosphatase for different phospho-serines and -threonines in the cell, this difference completely changes the activity of the phosphatase against different signal transduction pathways important for cancer cell growth and survival.

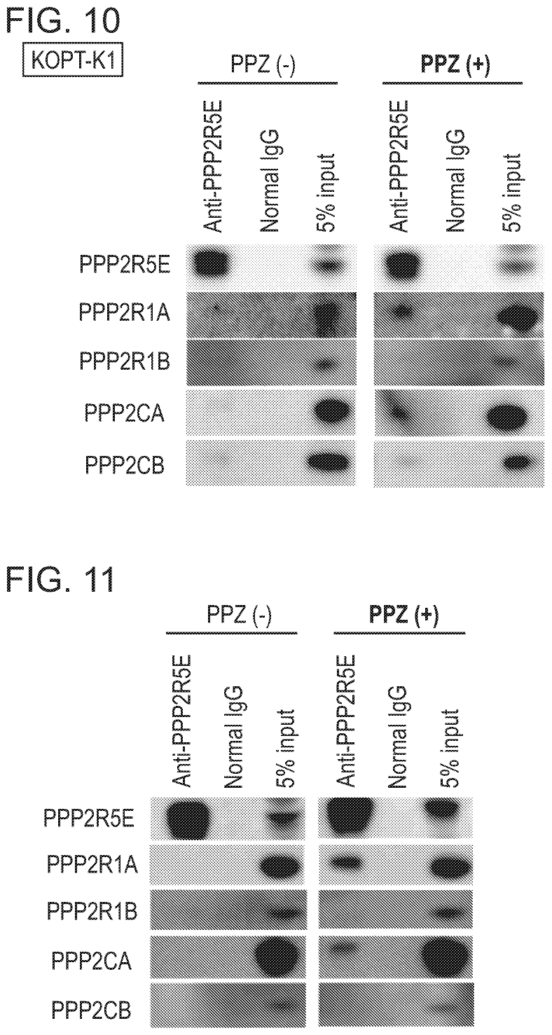

A similar IP-pulldown assay was conducted after immunoprecipitating with the anti-PPP2R5E antibody. As illustrated in FIG. 10, PPP2R1A and PPP2CA were shown to bind to PPP2R5E, but only after KOPT-K1 cells were treated with PPZ. Cells were treated with PPZ at 10 μM or control DMSO for 24 hours before lysed for protein extraction. Protein lysates were then co-immunoprecipitated with anti-PPP2R5E antibody and immunoblotted with antibodies uniquely-detecting each of the subunit of PP2A. The binding of PPP2CA and PPP2R1A to PPP2R5E was detected only in the PPZ-treated lysates.

To determine whether these three subunits would assemble into the PP2A holoenzyme, protein extracts from KOPT-K1 cells were examined. The lysates were assayed before and after the addition of PPZ to the cell lysate. Cell lysates were incubated with PPZ or DMSO vehicle at the room temperature for 1 hour, and then immunoprecipitated with the anti-PPP2R5E antibody. The binding of PPP2CA and PPP2R1A to PPP2R5E was again detected only in the PPZ-treated lysates (FIG. 11). Cells were first lysed for protein extraction, then protein lysates were incubated with PPZ at 10 μM (PPZ+) or DMSO control (PPZ−) for one hour at room temperature before co-immunoprecipitation with the anti-PPP2R5E antibody. The indicated subunits of PP2A in the immunoprecipitants were detected by western blotting. The results show that PPZ did not have to be added to living cells, but can be added after cells have been lysed to the cell lysate, and it was still able to initiate the formation of a specific trimeric PP2A complex containing PPP2CA, PPP2R1A and PPP2R5E.

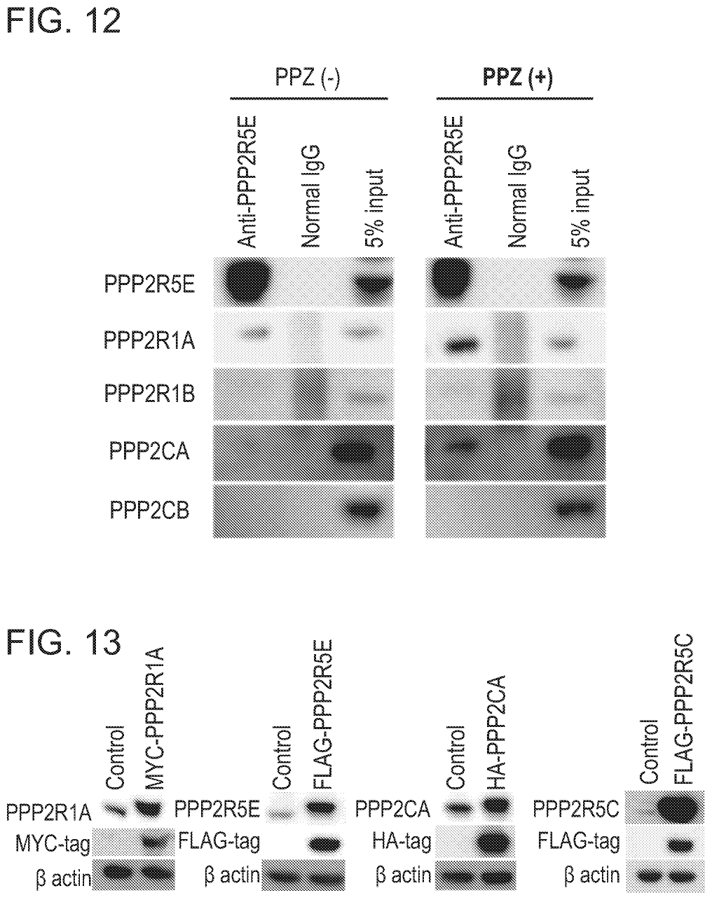

PPZ-mediated assembly of the PP2A heterotrimeric holoenzyme was also shown in SUPT-13 cells, which is a different T-ALL cell line, indicating that our findings apply broadly to T-ALL cells that are sensitive to PPZ treatment (FIG. 12). SUPT-13 cells were treated with PPZ at 10 μM or control DMSO for 24 hours before lysed for protein extraction. Protein lysates were then co-immunoprecipitated with anti-PPP2R5E antibody and immunoblotted with antibodies uniquely detecting each of the subunit of PP2A. The binding of PPP2CA and PPP2R1A to PPP2R5E was detected only in the PPZ-treated lysates.

To determine whether PPZ acts exclusively on these three subunits to mediate the assembly of PP2A, or if other proteins expressed by T-ALL cells are also required, C-terminal tagged cDNAs of each subunit of PP2A were expressed in Hi5 insect cells using a baculovirus vector and purified the subunit proteins using columns with antibodies against the tags bound to agarose beads (FIG. 13 and FIG. 14). To achieve the results in FIG. 13, the cDNA constructs encoding the tagged PP2A subunits were subcloned into pcDNA3 expression vectors. HEK293T cells were transiently transfected with pcDNA3-MYC-PPP2R1A, pcDNA3-FLAG-PPP2R5E or pcDNA3-FLAG-PPP2R5C and pcDNA3-HA-PPP2CA, then lysed for protein extraction. The PP2A complex containing MYC-PPP2R1A, FLAG-PPP2R5E and HA-PPP2CA was detected by western blotting of the transfected HEK293T cells. To achieve the results in FIG. 14, the cDNA constructs of tagged subunits were subcloned into pAC8 baculovirus expression vectors for insect cell expression. pAC8-MYC-PPP2R1A, pAC8-FLAG-PPP2R5E/PPP2R5C or pAC8-HA-PPP2CA were co-transfected with linearized baculovirus DNA into Sf9 insect cells for baculovirus production. Hi5 insect cells were infected with the baculovirus preparations for protein expression and the expressed subunit proteins were purified using a protein purification kit (MBL). The purity of the products was examined by Coomassie stain.

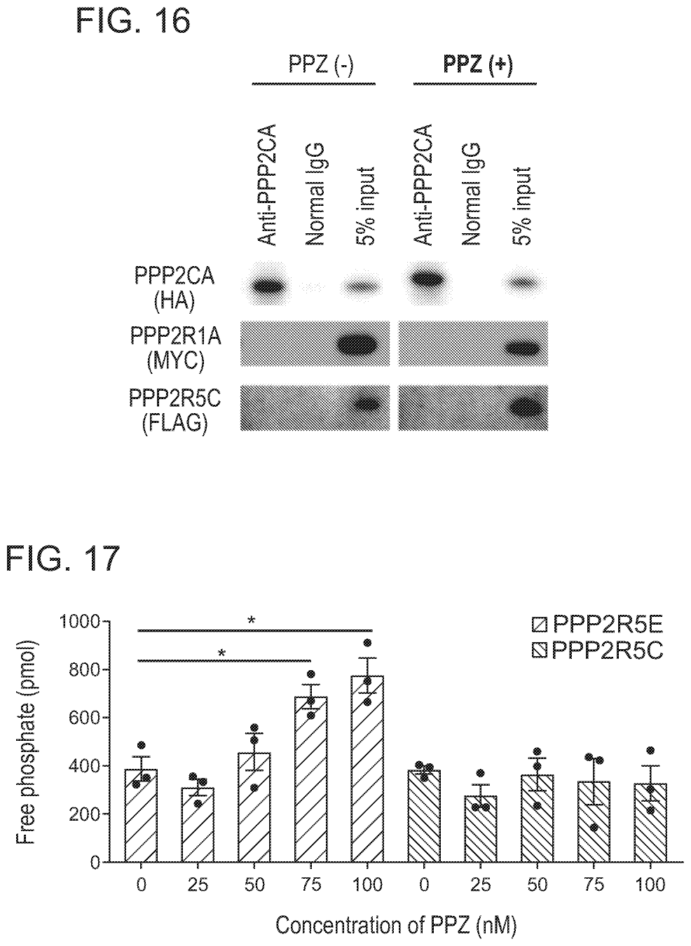

As in T-ALL cells, the association of PPP2R1A, PPP2CA and PPP2R5E was observed when these subunit proteins were incubated with PPZ (FIG. 15), while substitution of PPP2R5C for PPP2R5E abrogated this PPZ-mediated association of a complex (FIG. 16). To achieve the results in FIG. 15, a mixture of the three subunits was incubated either with PPZ at 10 μM (PPZ+) or control DMSO (PPZ−) for one hour at room temperature, then immunoprecipitated with anti-PPP2CA antibody. Indicated subunits of PP2A in the pulled-down products were detected by western blot. PPZ induced the formation of a trimeric PP2A complex from these three purified proteins, without any other mammalian proteins present in the lysate and very little residual insect proteins, as shown in the Coomassie-stained gel in FIG. 14.

For the results in FIG. 16, each of the three subunits was incubated either with PPZ at 10 μM (PPZ+) or control DMSO (PPZ−) for one hour at room temperature, then pulled down with anti-PPP2CA antibody. Indicated subunits of PP2A in the immunoprecipitated products were detected by western blot. PPZ did not induce the formation of a trimeric PP2A complex when FLAG-tagged PPP2R5C is substituted for FLAG-tagged PPP2R5E. Thus, PPZ specifically induced the formation of complexes containing the PPP2R5E subunit.

PPP2R5C is another B′ subunit that is highly-expressed in T-ALL cells (FIG. 7), demonstrating the specificity for PPP2R5E of the PPZ-nucleated PP2A holoenzyme. Consistent with these findings, the phosphatase activity of PP2A was increased upon PPZ treatment when PP2A was assembled from the purified PPP2R1A, PPP2CA and PPP2R5E subunits, while its activity was unchanged from control when PPP2R5C was substituted for PPP2R5E (FIG. 17). PPP2R1A, PPP2CA and PPP2R5E or PPP2R5C produced in insect cells were incubated with PPZ at the indicated concentrations for one hour at room temperature before the phosphatase activity of PP2A was measured. The data is presented as mean±s.d. (n=3, biological replicates). *P<0.05 by student's t-test. Phosphatase activity increased with increasing concentrations of PPZ. Subunit mixtures substituting PPP2R5C for PPP2R5E did not show PPZ-dependent phosphatase activity (blue bars), indication specificity PPZ to activate complexes containing PPP2R5E.

### Example 6

### Examination of Target Engagement Specificity of PPZ and iPAP1 (for Improved Heterocyclic Activator of PP2A) to PP2A Subunits in KOPT-K1 Cells by Cellular Thermal Shift Assay

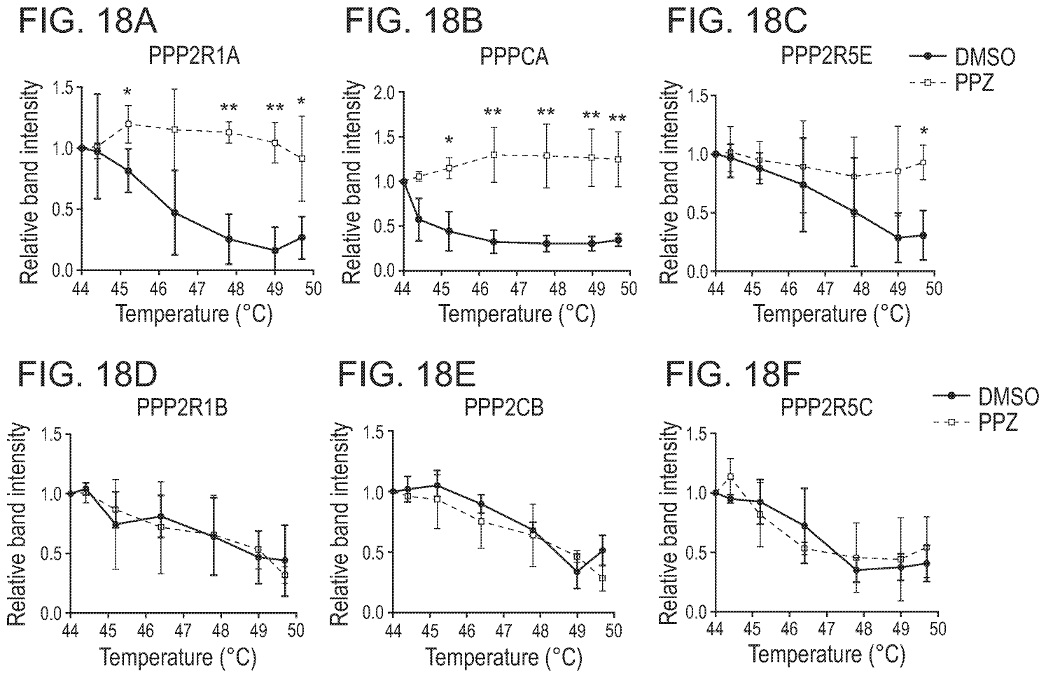

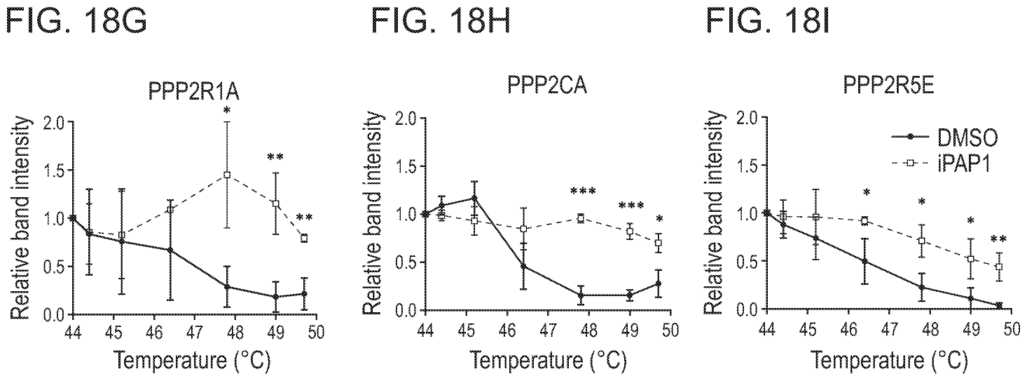

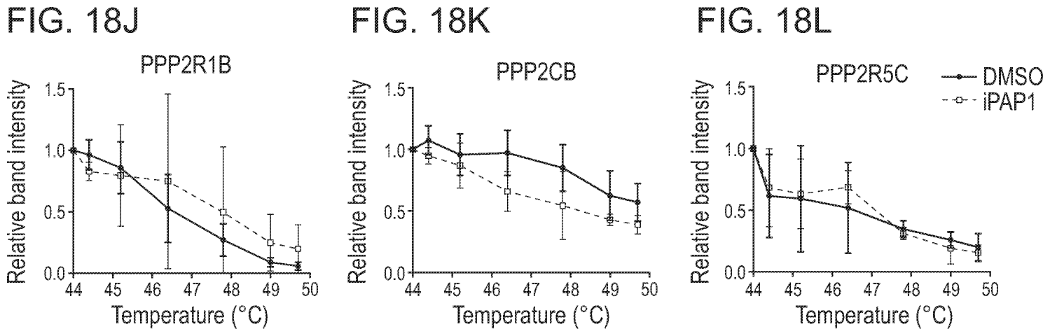

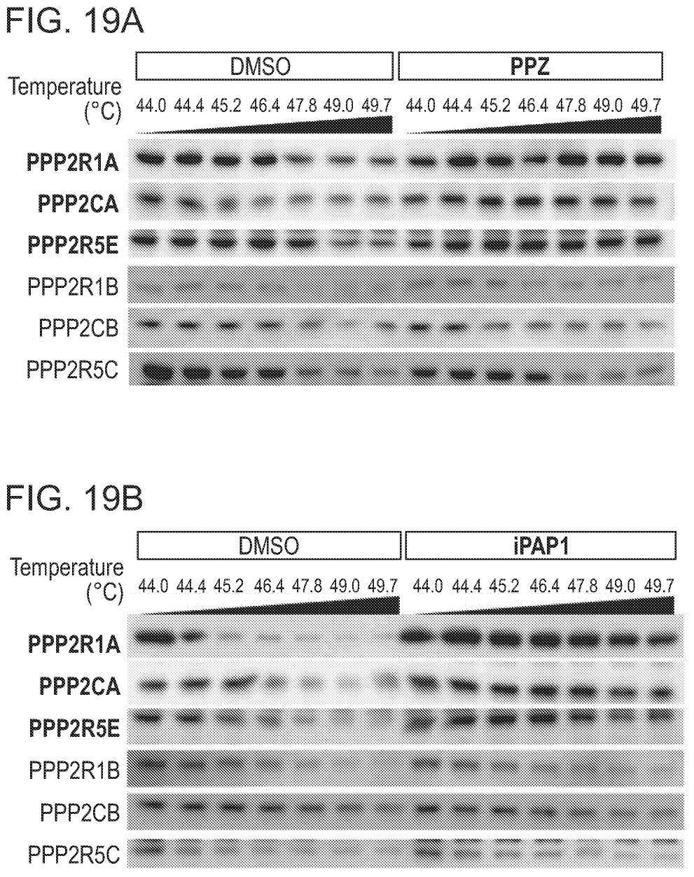

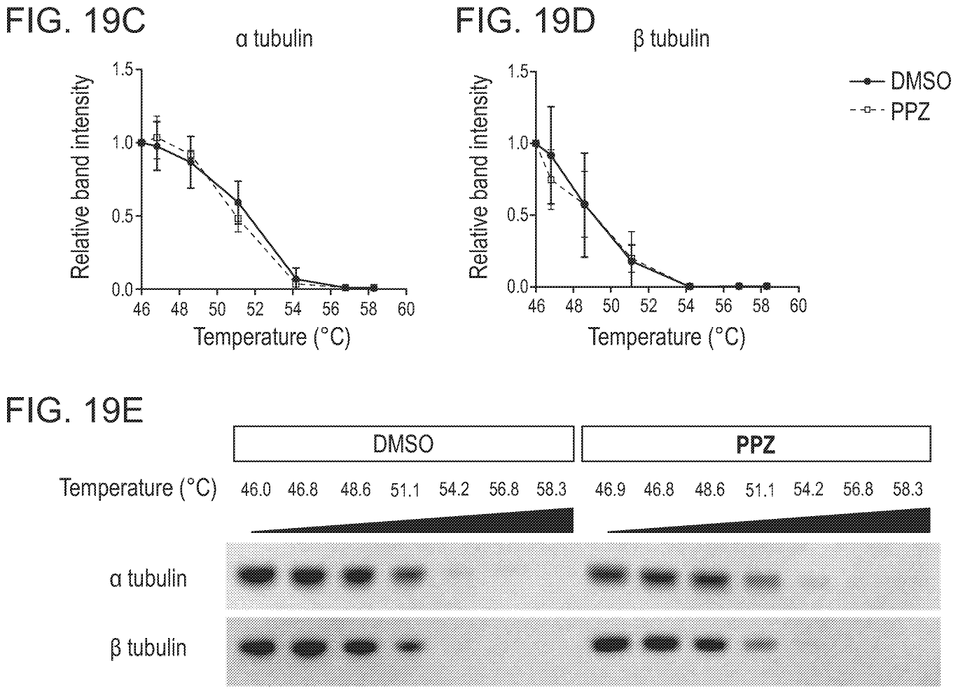

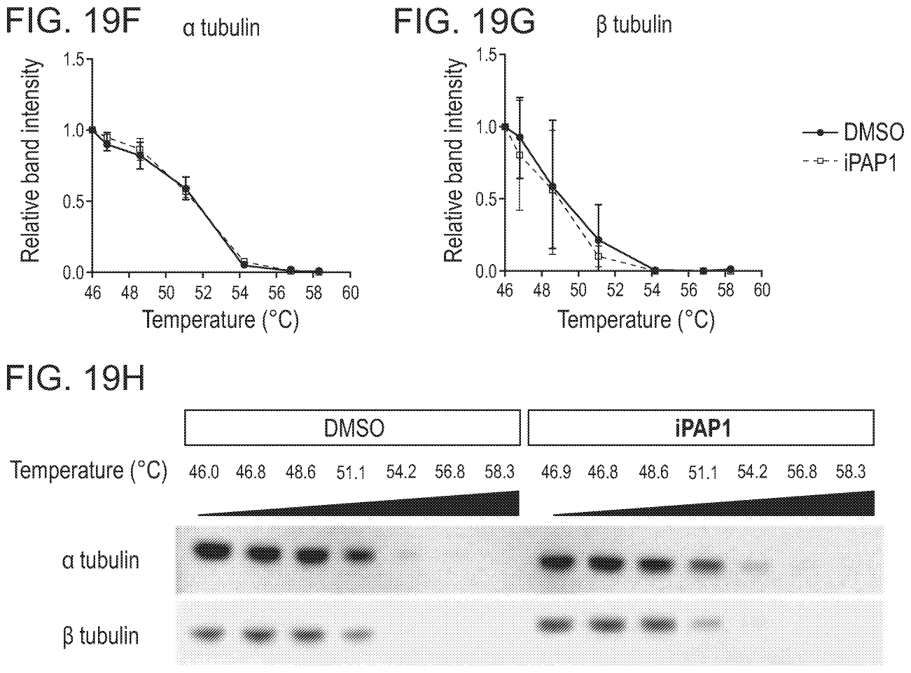

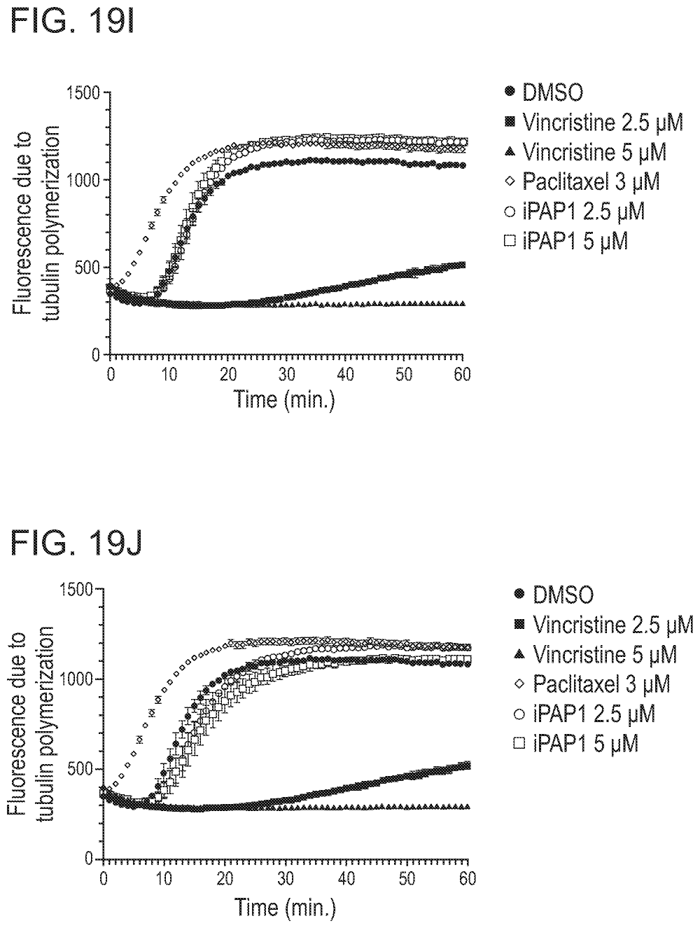

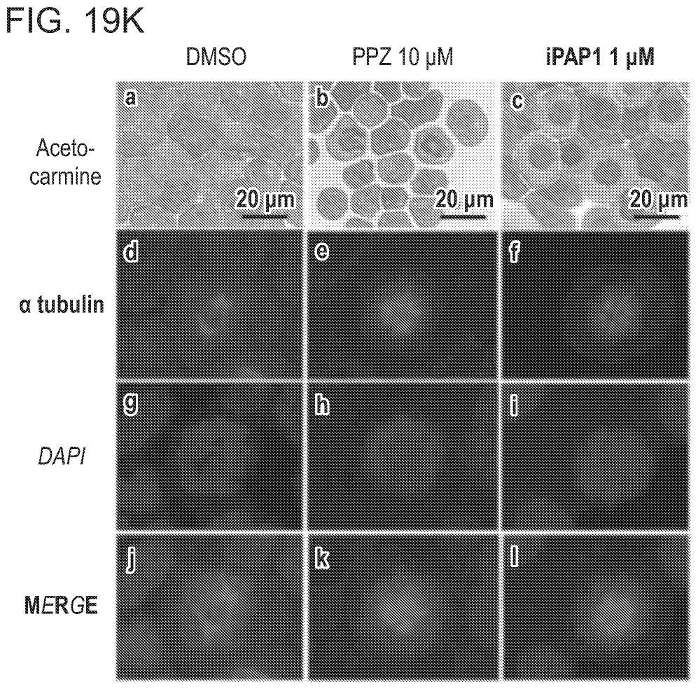

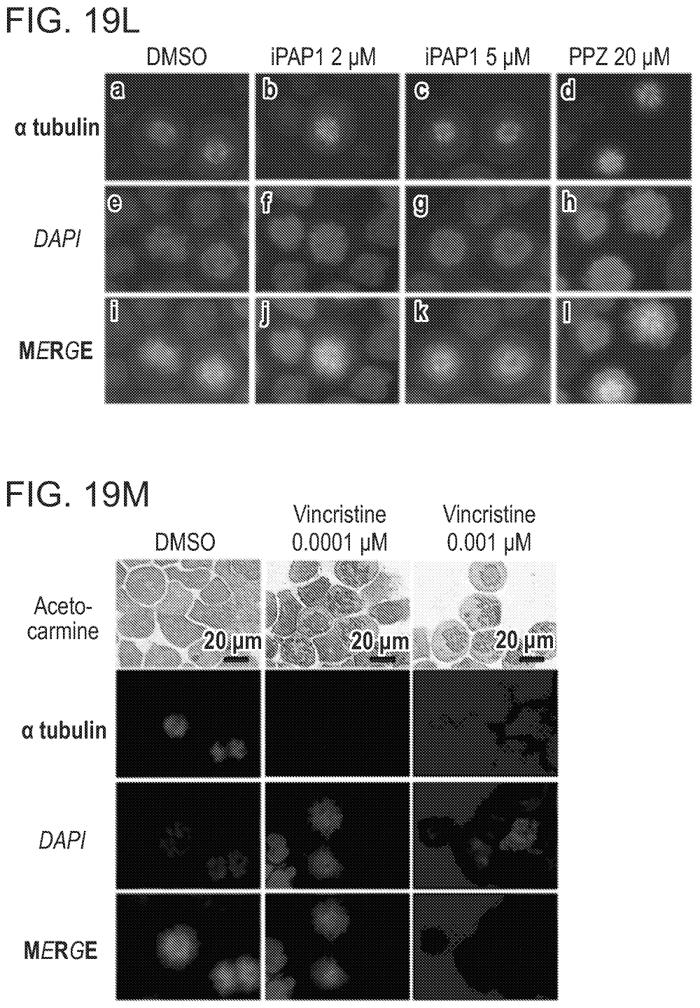

To consolidate the robustness of our findings, the specificity of target engagement of PPZ to the subunits of PP2A in KOPT-K1 cells was examined with Cellular Thermal Shift Assay (CETSA) (Tsuyoshi et al., Scientific Reports 7: 13000 (2017)). Cell lysates extracted from KOPT-K1 cells were incubated either with PPZ at 10 μM or control DMSO for one hour at room temperature before heat treatment at various temperatures. The cell lysates were then ultra-centrifuged to precipitate the aggregated pool of the protein, followed by quantification of the remaining soluble protein fraction by immunoblotting with antibodies specific to each of the PP2A subunits. In principle, subunits of PP2A bound by PPZ acquire thermal stabilization and remain in the fraction corresponds to non-denatured folded proteins. Using this assay, significantly enhanced stabilization of PPP2R1A, PPP2CA and PPP2R5E was found in PPZ-treated cells over control. In contrast, this heat-resistance was not acquired in PPP2R1B, PPP2CB and PPP2R5C, indicative of specifically preferred interaction of PPZ with PPP2R1A, PPP2CA and PPP2R5E in KOPT-K1 cells (FIG. 18A-FIG. 18F and FIG. 19A). The degree of protection from thermal denaturation provides a quantitative measurement of target engagement for a drug like PPZ, which is shown in FIG. 18A-FIG. 18F to protect PPP2R1A, PPP2CA and PPP2R5E, but not PPP2R1B, PPP2CB or PPP2R5C.

As illustrated in FIG. 18A-FIG. 18C, the three subunits underwent increasing thermal degradation with time without PPZ, but showed much less degradation when incubated with PPZ, indicating stabilization due to formation into a complex. However, other PP2A subunits that were not induced to form a trimeric complex by PPZ showed identical levels of increasing thermal degradation when incubated with or without PPZ, illustrating the applicability of this assay to reveal thermal stability after complex formation by PP2A subunits (FIG. 18D-FIG. 18F). These results collectively indicate that PPZ specifically facilitated the assembly of PPP2R1A, PPP2CA and PPP2R5E into a PP2A heterotrimeric holoenzyme in T-ALL cells and triggered its activity as a phosphatase.

A similar experiment was set up to examine target engagement specificity of iPAP1 to PP2A subunits in KOPT-K1 cells (FIG. 18G-FIG. 18L). The results show that iPAP1 also caused PP2 complex formation, resulting in protection of the PP2A subunits from thermal degradation similar to PPZ. The three subunits underwent increasing thermal degradation with time without iPAP1, but showed much less degradation when incubated with iPAP1, indicating stabilization due to formation into a complex (FIG. 18G-FIG. 18I). However, other PP2A subunits that were not induced to form a trimeric complex by iPAP1 showed identical levels of increasing thermal degradation when incubated with or without iPAP1, illustrating the applicability of this assay to reveal thermal stability after complex formation by PP2A subunits (FIG. 18J-FIG. 18L). A western blot of the KOPT-K1 cell lysates is illustrated in FIG. 19B.

The cellular thermal shift assay (CETSA) curves for KOPT-K1 cell lysates with and without the addition of PPZ after incubation for 3 minutes are shown in FIG. 19C and FIG. 19D. α and β tubulins showed identical levels of increasing thermal degradation when incubated with or without PPZ, indicating that tubulins did not directly bind to or interact with PPZ. Data are presented as means±s.d. (n=3, biological replicates). The quantitation of levels α and β tubulins detected by specific antibodies was determined using western blotting (FIG. 19E). Alpha (α) and tubulin levels were quantified during CETSA by Image J software of KOPT-K1 cell lysates treated or untreated with iPAP1 at various temperatures for 3 minutes. These results, which were used to produce the graphs in FIG. 19C-FIG. 19D, show PPZ did not protect α or β tubulins from thermal degradation, indicating that this assay did not detect direct interaction of PPZ with α or β tubulins. Taken together, these results indicate that PPZ did not cause cell cycle arrest in prometaphase by directly interacting with tubulins.

The CETSA curves for KOPT-K1 cell lysates with and without the addition of iPAP1 after incubation for 3 minutes are shown in FIG. 19F and FIG. 19G. Alpha (α) and β tubulins showed identical levels of increasing thermal degradation when incubated with or without iPAP1, indicating that this assay did not detect direct interaction of iPAP1 with α and β tubulins do not bind to or interact with iPAP1. Data are presented as means±s.d. (n=3, biological replicates). The quantitation of levels α and β tubulins detected by specific antibodies using western blotting (FIG. 19H). α and β tubulin levels were quantified during CETSA by Image J software of KOPT-K1 cell lysates treated or untreated with iPAP1 at various temperature for 3 minutes. The results were used to generate the graphs in FIG. 19F-FIG. 19G and they indicate that iPAP1 did not protect α or β tubulins from thermal degradation, indicating that iPAP1 did not directly interact with α or β tubulin. Taken together, these results indicate that iPAP1 did not cause cell cycle arrest in prometaphase by directly interacting with α or β tubulin. PPZ and iPAP1 kill T-ALL cells and blocked their cell cycle progression by activating the phosphatase activity of a specific PP2A trimeric complex (PPP2R5E, PPP2R1A, and PPP2CA). The data are different from the results of another group suggesting that these drugs act by inhibiting tubulin polymerization (Prinz et al., J. Med. Chem. 54:4247-4263 (2011); Prinz et al., J. Med. Chem. 60:749-766(2017)).

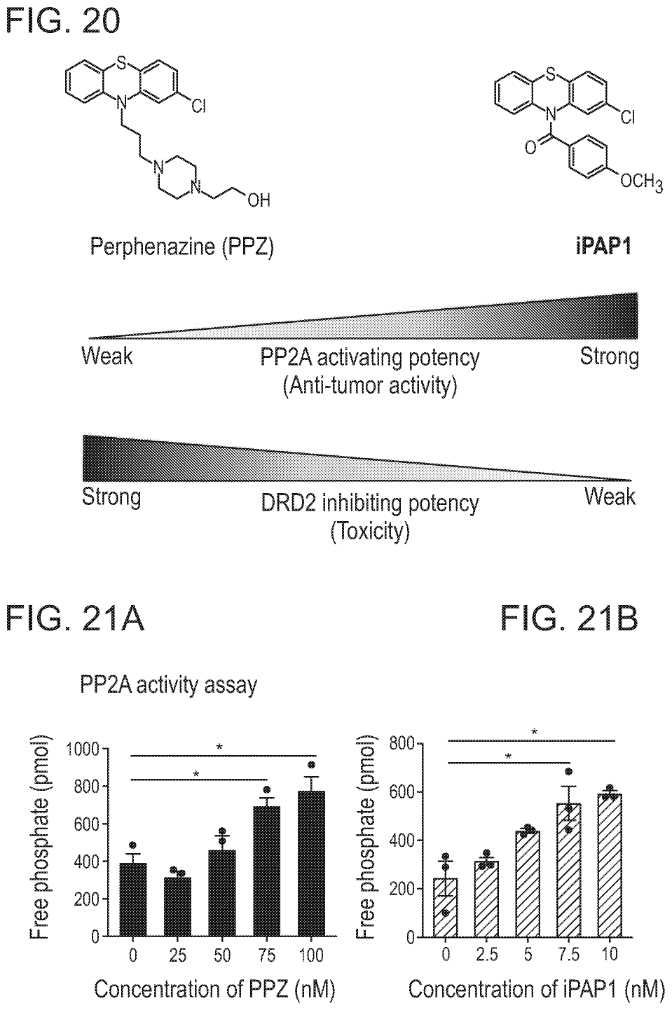

Prinz and coworkers have reported the synthesis of 53 N-benzoylated phenoxazines and phenothiazines, as well as their S-oxidized analogues, concluding that some of these compounds interfere with tubulin depolymerization (Prinz et al., J. Med. Chem. 54:4247-4263 (2011); Prinz et al., J. Med. Chem. 60:749-766(2017)).Thus, PPZ and iPAP1 were tested biochemically using a tubulin polymerization assay with highly purified tubulin from porcine brain (see, Example 29). In these assays, inhibition of tubulin polymerization was not detected at concentrations of up to 2.5 or 5 μM for either PPZ or iPAP1, while vincristine markedly inhibited tubulin polymerization at 3 μM (FIG. 19I-FIG. 19J). To directly observe the effects of these compounds in treated T-ALL cells, microtubules were tested in cytospin preparations of living KOPT-K1 cells treated with PPZ at 10 μM (FIG. 19K) and 20 μM (FIG. 19L) and iPAP1 at 2 μM and 5 μM (FIG. 19L), observing prometaphase arrest at the monopolarity stage reflecting activated PP2A under each condition, with prominent microtubules radiating from central DAPI-stained chromatin in a star-shaped pattern. By contrast, in studies of KOPT-K1 cells incubated with 0.001 and 0.0001 μM vincristine as a positive control, the mitotic cells contained untethered DAPI-stained condensed chromosomes, without detectable microtubules (FIG. 19M). Thus, neither iPAP1 nor PPZ was found to affect microtubule assembly, as assessed by both in vitro assays and immunofluorescence assays in living cells.

### Example 7

### Bioactivities of PPZ and its Analog, iPAP1

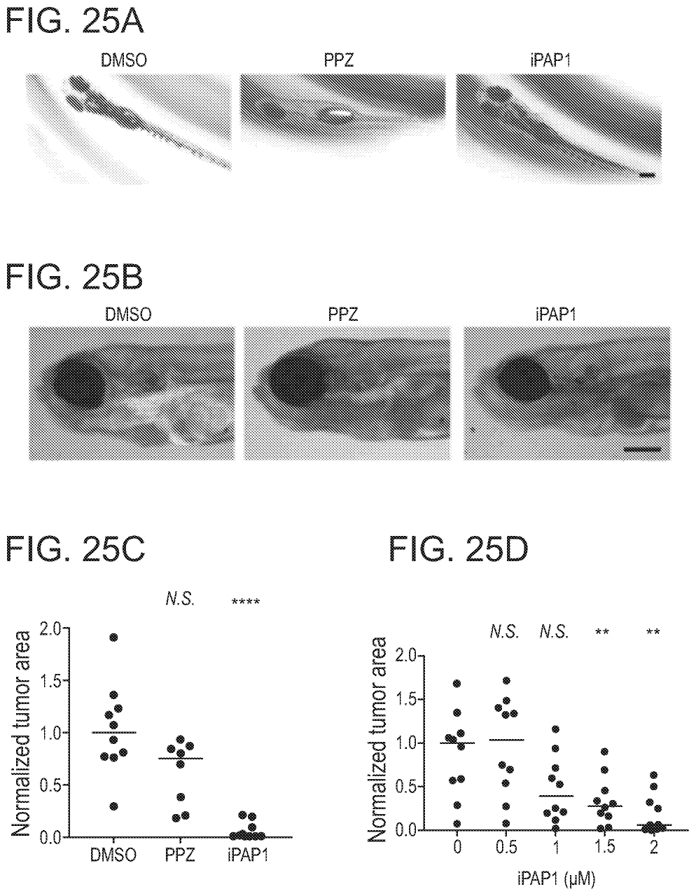

An image illustrating the unique bioactivities of perphenazine (PPZ) and its analog, iPAP1, is provided in FIG. 20. Biochemical assays showed that iPAP1 (also known as Z56843374, P-889442 and 14B) potently activated phosphatase activity of protein phosphatase 2A (PP2A) and induced apoptosis in T-ALL cells but lacked the ability to bind and inhibit dopamine receptor D2. These are the qualities are important in the identification of PP2A analogs that more potently kill cancer cells without causing movement disorders that have prevented the use of PPZ for anti-cancer treatment. (See, FIG. 20-FIG. 23B.) Further studies have now implicated a series of iPAP1-responsive cancers such as those represented by the responsive cell lines shown in FIG. 24. Consistent with its lack of dopamine receptor D2 inhibitory activity, this PPZ analog did not induce movement disorders in a zebrafish model system (FIG. 25), which is important because prior to the invention described herein, movement disorders limited the usefulness of PPZ as an anti-tumor drug in humans.

### Example 8

### Phosphatase Activity of PP2A via the PP2A Immunoprecipitation Phosphatase Assay Kit

Phosphatase activity of PP2A was measured using the PP2A Immunoprecipitation Phosphatase Assay Kit (Merck Millipore®) (FIG. 21A-FIG. 21B). Assays were conducted using purified A, B and C subunits (200 ng each), incubated with the indicated concentrations of PPZ, iPAP1 or equivalent amount of DMSO for one hour at room temperature. The proteins were subsequently incubated with protein A agarose slurry and 4 μg of anti-PPP2CA antibody (Merck Millipore®, #05-421, clone 1D6) at 4° C. with constant rocking for one hour. Agarose-bound immune complexes were collected, then washed with 700 μl TBS (3 times) and 500 μl Ser/Thr buffer (final wash), before resuspending them in 20 μl Ser/Thr buffer. A phosphopeptide (amino acid sequence: K-R-pT-I-R-R) was added as a substrate for PPP2CA, and samples were incubated at 30° C. in a shaking incubator for 10 minutes. Supernatants (25 μl) were then transferred onto 96-well plate, and released phosphate was measured by adding 100 μl malachite green phosphate detection solution. Absorbance was read by SpectraMax® M5 Multi-Mode Microplate Reader (Molecular Devices LLC) at 650 nm. Phosphate concentrations were calculated from a standard curve generated from serial dilutions of standard phosphate solution (0-2,000 pmol). Left panel shows the results with PPZ and right panel with iPAP1. iPAP1 showed equivalent PP2A activation activity at ˜10 times lower concentrations compared to PPZ. *P<0.05 by student's t-test.

### Example 9

### Dopamine Receptor D2 Inhibition Assay

The human dopamine receptor D2 (DRD2) and modified murine Gq5i cDNAs were inserted into pcDNA3 expression vectors. After verifying the PCR products by DNA sequencing, DRD2, Gq5i and the PathDetect pSRE-Luc Cis-Reporter Plasmid (#219080, Agilent Technologies) were transfected to HEK293T cells. As previously reported, Gq5i enables Gi/o-coupled receptor activity to be detected using a serum response element (SRE)-luciferase reporter gene (See, Conklin et al., Nature 363(6426):274-6 (1993); Al-Fulaij et al., J. Pharmacol. Exp. Ther. 321(1):298-307 (2007)). These cells were starved in FCS-free DMEM medium for six hours before incubating them with 2 μM dopamine hydrochloride (Sigma Aldrich) together with each of the compound at 0, 0.5, 1, 2 and 4 μM for three hours. The reporter activity was measured using Pierce™ Firefly Luciferase Glow Assay Kit (Thermo Fisher Scientific). Luminescence was monitored by SpectraMax® M5 Microplate Reader (Molecular Devices LLC). While PPZ showed strong inhibitory activity on DRD2 at 0.5 μM, iPAP1 showed no activity up to 4 μM. The results of the DRD2 inhibition assay used in this invention demonstrate that iPAP1 lacked measureable inhibition of dopamine receptor D2 and was more than 100 fold-less active in inhibiting DRD2 than PPZ when tested at multiple molar concentrations (FIG. 22).

As shown in FIG. 21A-FIG. 21B and FIG. 22, these assays reveal that iPAP1 activated PP2A at ˜10 fold lower molar concentrations that PPZ, and that iPAP1 had basically unmeasurable DRD2 inhibitory activity, definitely less than 1% of the activity of PPZ. These or similar biochemical assays using the purified PPP2R1A, PPP2CA and PPP2R5E proteins produced in insect cells for PP2A activity and a reporter to reveal DRD2 inhibition are critical, because they allow identification of ideal phenothiazine analogs for cancer treatment with optimal activation of the PP2A phosphatase, but that do not inhibit DRD2. Testing each phenothiazine analog with these or similar biochemical assays is key to this invention. iPAP1 illustrated some desired properties in that it was much more active than PPZ in activating PP2A activity (active at ˜10 fold lower concentrations) and did not show measurable DRD2.

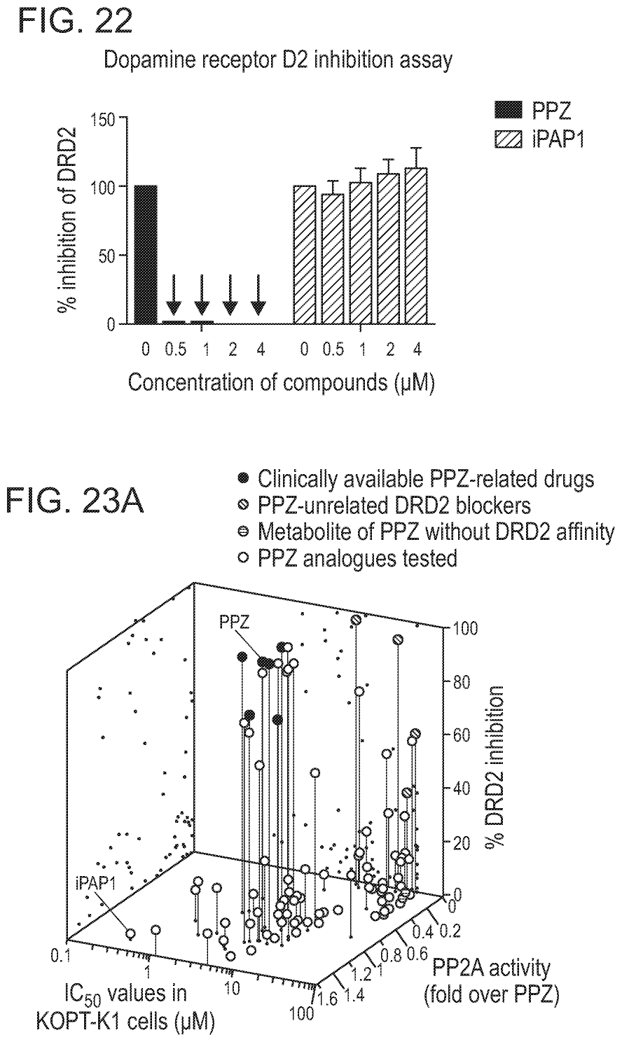

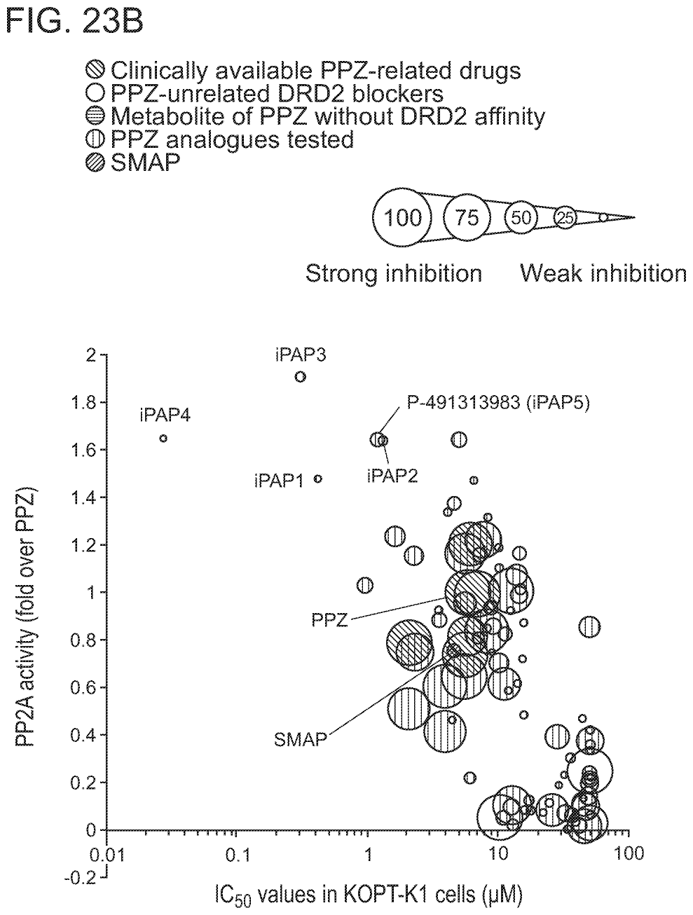

Some known drugs that have been tested so far are shown in FIG. 23A, which shows the results of testing for these two properties as well as the IC₅₀ against KOPTK1 cells after 3 days of treatment with each drug (see, Example 9). Of the compounds shown in FIG. 23A, IPAP1 had the lowest IC₅₀ value, the highest PP2A activation potential compared to PPZ and the least inhibition of DRD2 signaling, indicating that of these compounds, it has the most favorable properties for the treatment of human cancer. This approach, as described herein, is another aspect of this invention as is could easily lead to the identification of other analogs of phenothiazines that activate PP2A at even lower molar concentrations than iPAP1 and still do not inhibit DRD2. Indeed, an active search for such analogs of phenothiazines like PPZ is being conducted using these two crucial biochemical assays to assess candidate molecules.

FIG. 23B is a diagram showing the relationships among the key three parameters shown in FIG. 23A, including compounds iPAP2, iPAP3 and iPAP4, in addition to PPZ, iPAP1, and the other compounds shown in FIG. 23A. The X and Y axes represent the antileukemic potency and PP2A activation capacity respectively. The percent inhibition of the dopamine receptor D2 examined in HEK293T cells is represented by the size of the spheres, where the larger spheres indicate the stronger inhibitory potential. Of these drugs, IPAP4 had the lowest IC₅₀ value of 30-40 nM (˜10 fold lower than iPAP1 and ˜100 fold lower than PPZ), a relatively high PP2A activation potential compared to PPZ and very low inhibition of DRD2 signaling, making it the compound with the most favorable properties of the ones analyzed for treatment of human cancer. iPAP4 had the lowest IC₅₀ value against KOPTK1 cells even though its relative ability to activate PP2A was less than iPAP3. iPAP4 may be more permeable to cells or have other favorable properties that make it more active in killing living T-ALL cells. FIG. 23B also shows SMAP lacked DRD2 inhibitory activity and was much less potent than iPAP1, iPAP2, iPAP3 or iPAP4 in phosphatase activity and had an IC₅₀ value against KOPT-K1 cells that is orders of magnitude less potent than IPAP1, iPAP2, iPAP3 or iPAPP4 (IC₅₀ values of ˜5 micromolar for SMAP, ˜400 nanomolar for iPAP1, ˜1 micromolar for iPAP2, ˜300 nanomolar for iPAP3 and ˜30 nanomolar for iPAP4).

For IC₅₀ calculation, KOPT-K1 cells were treated with each of the compounds at various concentrations for 72 hours, followed by the determination of viable cell numbers with PrestoBlue® cell viability reagent. For the PP2A phosphatase activity assay, KOPT-K1 cells were treated with each of the compounds at 10 μM for 3 hours before phosphatase activity was measured. Dopamine D2 receptor activity was monitored in HEK293T cells coexpressing the dopamine D2 receptor, modified G protein and SRE luciferase reporter. Cells were treated with each of the compound at 10 μM for 3 hours, then lysed for the luciferase reporter assay.

### Example 10

### PP2A and DRD2 Inhibition with PPZ, iPAP1 and Analogs Thereof

Three-dimensional plots of PPZ analogs representing their inhibitory concentration 50 (IC₅₀) values in a T-ALL cell line (KOPT-K1 cells), as well as PP2A activation and DRD2 inhibition potentials (FIG. 23A). For IC₅₀ calculations, KOPT-K1 cells were treated with each of the compound at various concentrations for 72 hours, then their viability was examined by PrestoBlue® Cell Viability Reagent (Thermo Fisher Scientific). For PP2A phosphatase activity assay, KOPT-K1 cells were treated with each of the compound at 10 μM for three hours before the activity of PP2A was quantified using PP2A Immunoprecipitation Phosphatase Assay Kit (Merck Millipore®). DRD2 activity was monitored in HEK293T cells expressing DRD2, modified G protein and SRE luciferase reporter. Cells were treated with each of the compounds at 10 μM for three hours, then lysed for luciferase reporter assay. Totally 82 commercially available PPZ analogs, as well as six clinically approved phenothiazines that are structurally related to PPZ (PPZ, fluphenazine, chlorpromazine, prochlorperazine, thioridazine and trifluoperazine), three DRD2 inhibitors that are structurally unrelated to PPZ (sulpiride, domperidone, olanzapine and clozapine), and two metabolites of PPZ that do not bind to DRD2 (perphenazine sulfoxide and 2-chlorphenothiazine) were tested. The biochemical assays were performed as indicated in Examples 7 and 8.

As shown pictorially in FIG. 23A, each analog of PPZ had different combinations of potency in terms of i) IC₅₀ values obtained after treating cells from the T-ALL cell line KOPT-K1 for 72 hours, and ii) PP2A activation potency of each compound when added to KOPTK1 cell lysates, and iii) inhibitory concentration of DRD2 signaling examined in HEK293T cells. The clinically available phenothiazines cluster together in this three-dimensional display as represented by the red balls, with moderate potency for PP2A activation and with high inhibitory potency against DRD2. For DRD2 blockers that are structurally unrelated to PPZ, four drugs were tested in this experiment, represented by the green balls, with two strong inhibitors (sulpiride and domperidone) and two moderate inhibitors (clozapine and olanzapine). As expected, they showed moderate to high inhibitory potency against DRD2 in our assay, reflecting their known affinity for this particular receptor (green balls). Each of these four DRD2 inhibitors did not stimulate PP2A phosphatase activity. Two metabolites of PPZ known to lack affinity for dopamine D2 receptor showed very little inhibition of DRD2 or activation of PP2A (blue balls). By contrast, the analogs of PPZ tested with these assays showed diverse properties (yellow balls), with very different capacities for inhibitory activity of DRD2, PP2A activating potential and anti-tumor activity in T-ALL cells. The most promising analog of PPZ in these experiments (iPAP1) was 10 times more active for stimulating PP2A phosphatase activity and 10 times more active for mediating T-ALL cell killing, and at the same time had much less inhibitory activity against DRD2 compared to PPZ (˜1%). Another compound, iPAP5, also known as P-491313983, is the yellow ball next to iPAP1 in FIG. 23A.

The relationships among the key three parameters shown in FIG. 23A, including compounds iPAP2, iPAP3 and iPAP4, are illustrated in FIG. 23B. The X and Y axes represent the antileukemic potency and PP2A activation capacity respectively. The percent inhibition of the dopamine receptor D2 examined in HEK293T cells is represented by the size of the spheres, where the larger spheres indicate the stronger inhibitory potential. Of these drugs IPAP4 has the lowest IC₅₀ value of 30-40 nM (˜10 fold lower than iPAP1 and ˜100 fold lower than PPZ), a relatively high PP2A activation potential compared to PPZ and very low inhibition of DRD2 signaling, making it the compound with the most favorable properties of the ones analyzed for treatment of human cancer. iPAP4 has the lowest IC₅₀ value against KOPTK1 cells even though its relative ability to activate PP2A is less than iPAP3. iPAP4 may be more permeable to cells or have other favorable properties that make it more active in killing living T-ALL cells.

For IC₅₀ calculation, KOPT-K1 cells were treated with each of the compounds at various concentrations for 72 hours, followed by the determination of viable cell numbers with PrestoBlue® cell viability reagent. For the PP2A phosphatase activity assay, KOPT-K1 cells were treated with each of the compounds at 10 μM for 3 hours before phosphatase activity was measured. Dopamine D2 receptor activity was monitored in HEK293T cells coexpressing the dopamine D2 receptor, modified G protein and SRE luciferase reporter. Cells were treated with each of the compound at 10 μM for 3 hours, then lysed for the luciferase reporter assay.

### Example 11

### PRISM (Profiling Relative Inhibition Simultaneously in Mixtures) Analysis of Cell Viability After PPZ or iPAP1

A PRISM (Profiling Relative Inhibition Simultaneously in Mixtures) analysis of cell viability relative to DMSO control after treatment for 5 days with 5 μM concentration of PPZ or iPAP1 against 274 cancer cell lines from 39 distinct types of human cancers was conducted (Yu et al., Nat. Biotechnol. 34:419-23 (2016)). These cell lines were invariably more sensitive to iPAP1 than PPZ and cell lines as sensitive or more sensitive than T-ALL cells were found in many types of human cancers, including other hematologic malignancies, neuroblastoma, small cell lung cancer, lung adenocarcinoma, glioblastoma and breast carcinoma. In every cell line, iPAP1 was more active in cell killing than was PPZ. Many other human cell lines from hematologic malignancies and solid tumors were as sensitive as the most sensitive T-ALL cell lines to treatment with iPAP1. Cell lines with a relative viability below the dashed line at 0.5 have an IC₅₀ value for iPAP1 below 5 μM. This study illustrates the potentially wide applicability of iPAP1 and other similar PPZ analogs for the treatment of a wide variety of human cancers.

### Example 12

### Neurological Toxicity and Anti-Tumor Activity of iPAP1 In Vivo in Zebrafish

Representative free-swimming eight-day old zebrafish embryos after five days of treatment with DMSO (control), 5 μM PPZ or 2 μM iPAP1 are shown in FIG. 25A. Note that the embryos were not anesthetized. Scale bar: 0.1 mm. DMSO and iPAP1 treated embryos swam upright, while PPZ-treated embryos exhibited movement disorders and swam upside down or on their sides.

FIG. 25B shows representative eight-day old zebrafish embryos transplanted with T-ALL cells isolated from Tg(rag2:Myc; rag2:EGFP) zebrafish and treated for five days with DMSO (control), 5 μM PPZ or 2 μM iPAP1. PPZ showed some activity against T-ALL with reduced GFP signal while iPAP1 reduced the GFP signal at least 5 fold more than PPZ, reflecting greater T-ALL cell killing. Scale bar: 0.1 mm.

FIG. 25C shows the results of quantified GFP-positive leukemic area in zebrafish embryos treated with 5 μM PPZ or 2 μM iPAP1, compared to DMSO treatment (control), quantifying the increased T-ALL cell killing by iPAP1. (n=10 for DMSO and iPAP1, n=8 for PPZ)

FIG. 25D shows the results of quantified GFP-positive leukemic area in zebrafish embryos treated with serial dosage of iPAP1, compared to DMSO treatment, showing dose dependent increased T-ALL cell killing by iPAP1. (n=10) N.S.; not significant, **P<0.01 and ****P<0.0001 by two-tailed Welch's t-test.

### Example 13

### iPAP1 Actively Killed T-ALL Tumor Cells in Mice In Vivo Model without Showing PPZ-Related Neurological Toxicity

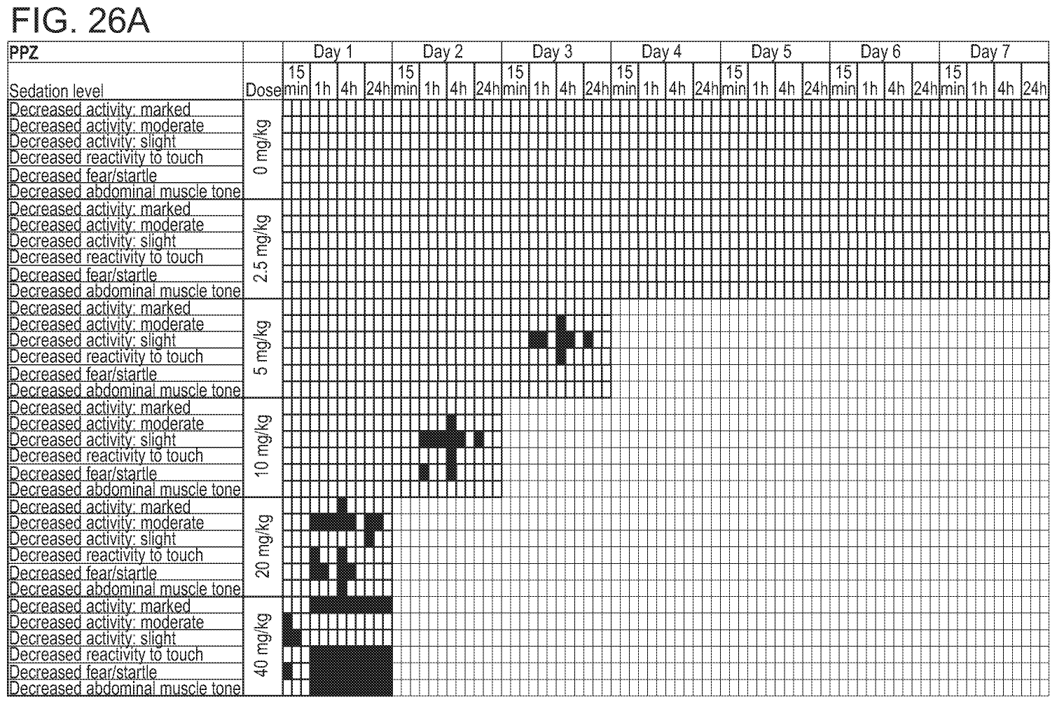

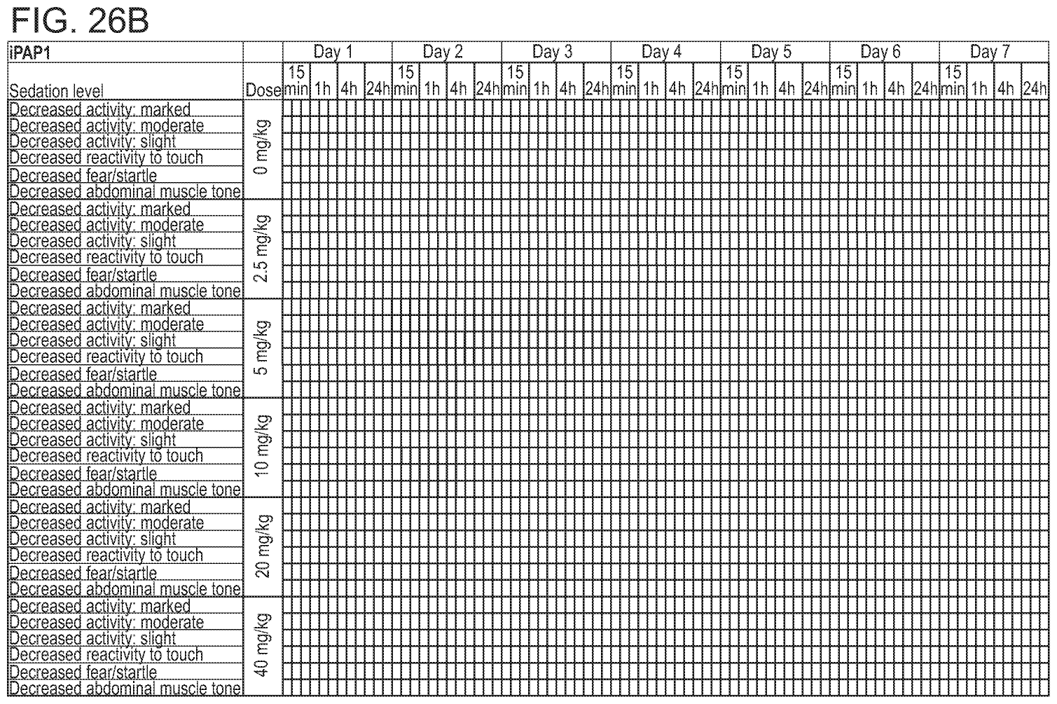

Dose-dependent neurological toxicity of PPZ and iPAP1 was tested in mice (FIG. 26A-FIG. 26B). PPZ and iPAP1 were each given per orally to eight-week-old female C57BL/6 mice every 24 hours (n=3 for each cohort). The mice were monitored for four recognized types of dopamine receptor D2 mediated toxicity, namely: i) general activity; ii) reactivity to touch; iii) fear/startle to sound; and iv) tone of abdominal muscle. Behavioral monitoring was done at 15 minutes, 1 hour, 4 hours and 24 hours after each treatment. PPZ and iPAP1 were each administered at 0, 2.5, 5, 10, 20 and 40 mg/kg body weight/dose, and iPAP1 was also administered at 80 mg/kg body weight/dose. During the one-week monitoring period after initial treatment, mice treated with PPZ of 5 mg/kg body weight/dose or more showed neurological toxicity, establishing the maximum tolerated dose as 2.5 mg/kg. Mice treated with iPAP1 did not show any neurological toxicity when administered up to 80 mg/kg body weight/dose.

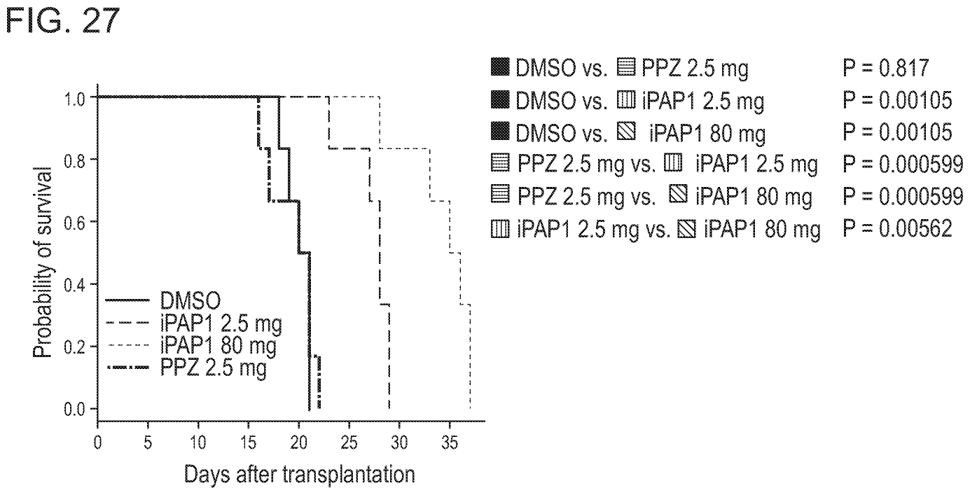

Anti-tumor activities of PPZ and iPAP1 tested in immunodeficient NSG (NOD/Scid/IL2Rγnull) mice xenotransplanted with KOPT-K1 cells are shown in FIG. 27. At day one, 1×10⁶ KOPT-K1 cells were intravenously injected into each mouse via its tail veins. At day 10, transplanted mice were randomly assigned to four treatment groups (DMSO, PPZ 2.5 mg/kg/day, iPAP1 2.5 mg/kg/day and iPAP1 80 mg/kg/day), and each of the treatment was started per orally, every 24 hours. Treatment with PPZ at its maximum tolerability dose (2.5 mg/kg/day) did not show any survival advantage over the control. By contrast, treatment with iPAP1 at 2.5 mg/kg/day significantly extended the overall survival period over control or PPZ treatment cohorts. Favorable effects on the overall survival in this T-ALL mice model was even more significant with high-dose iPAP1 treatment at 80 mg/kg/day.

### Example 14

### Cell Viability Tests with PPZ and iPAP1

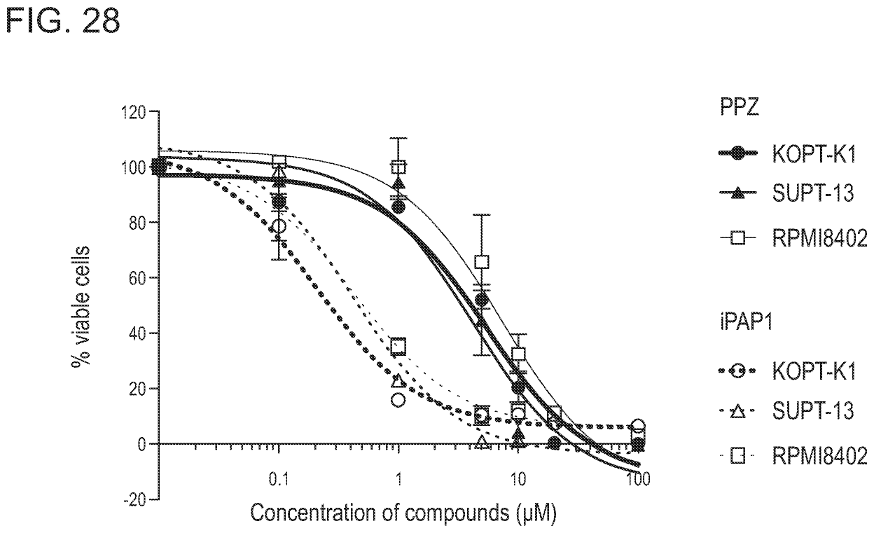

Dose-response curves of PPZ and iPAP1 in KOPT-K1, RPMI8402 and SUPT-13 cells (FIG. 28). Cells were treated with PPZ or iPAP1 at various concentrations for 72 hours, then their viability was examined. Viable cells are shown on the Y axis as a percent of the DMSO control cells. Data are presented as means±s.d. (n=3). Data are presented as means±s.d. (n=3, biological replicates). iPAP1 was about 10 times more active than PPZ for mediating T-ALL cell killing in each of the three different T-ALL cell lines tested. The IC₅₀ for iPAP1 is 200 to 400 nM for these cell lines.

### Example 15

### IC₅₀ of PP2A Activators in SUPT-13, KOPT-K1 and RPMI-8402 Cells

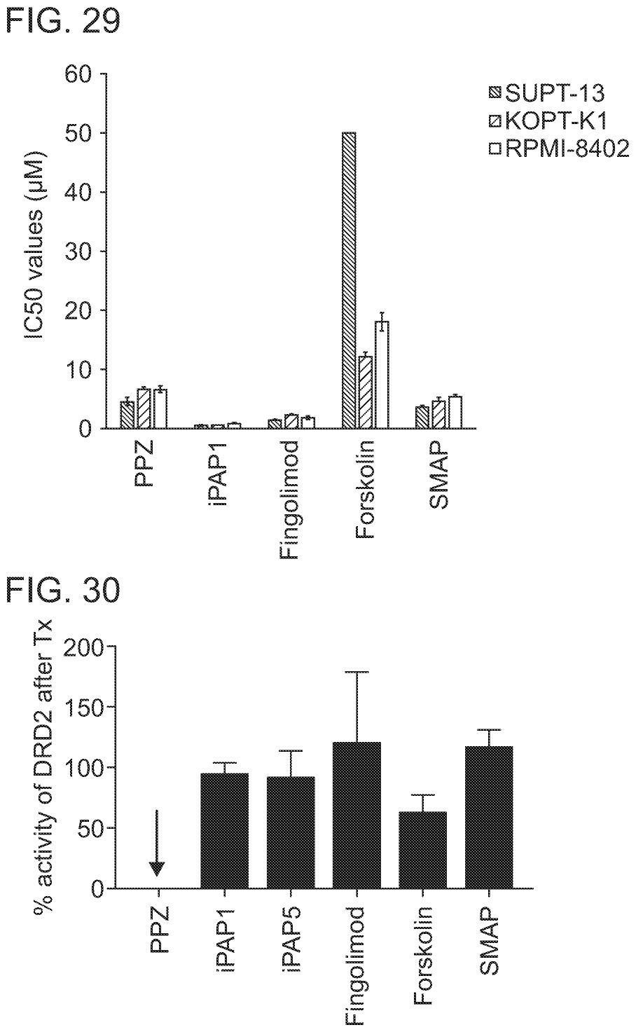

iPAP1 is more potent in inducing cell death in cancer cells than perphenazine, and the other three reported PP2A activators, forskolin, fingolimide and SMAP. (Perrotti and Neviani, Cancer Metastasis Rev. 27(2):159-68 (2008); Sangodkar et al., J. Clin. Invest. 127(6):2081-2090 (2007)). IC₅₀ values for available PP2A activators forskolin (Feschenko et al., J. Pharmacol. Exp. Ther. 302:111-8 (2002); Cristobal et al., Leukemia 25:606-14 (2011)), fingolimod (Oaks et al., Blood 122:1923-34 (2013)), small molecule activator of protein phosphatase (Sangodkar et al., J. Clin. Invest. 127:2081-2090 (2017)), PPZ and iPAP1 were determined. Among PP2A activating compounds tested, iPAP1 showed the most potent anti-tumor activity against three different T-ALL cell lines (SUPT-13, KOPT-K1 and RPMI-8402 cells). Data are presented as means±s.d. (n=3, biological replicates) (FIG. 29). iPAP1 is much more active (˜5 fold) than SMAP against T-ALL cells based on the IC₅₀.

Fingolimod is a clinical available immunosuppressant known to have some PP2A activator activity. Forskolin is an herbal supplement reported in 2002 to act by increasing cAMP levels (see, Prinz et al., J. Med. Chem. 54(12):4247-63 (2011)). SMAP, a drug developed and tested by Sangodkar (see, Sangodkar et al., J. Clin. Invest. 127(6):2081-90 (2017)), is a derivative of phenothiazine with the basic amine replaced with a neutral polar functional group. iPAP1 was the most active PP2A activator that has been identified in terms of killing T-ALL cells and was also the most active among any of the previously reported compounds described herein (FIG. 29). Significantly, iPAP1 was also the compound with the least level of DRD2 inhibition of dopamine receptor D2.

### Example 16

### DRD2 Activity Test with Various PP2A Activators

DRD2 activities after treatment with various PP2A activators, including iPAP1 and the second best compound in FIG. 23, P-491313983, were measured. The human dopamine receptor D2 and modified murine Gq5i cDNAs were inserted into pcDNA3 expression vectors. After verifying the PCR products by DNA sequencing, DRD2, Gq5i and the PathDetect pSRE-Luc Cis-Reporter Plasmid (#219080, Agilent Technologies) were transfected to HEK293T cells. As previously reported, Gq5i enables Gi/o-coupled receptor activity to be detected using a serum response element (SRE)-luciferase reporter gene (See, Conklin et al., Nature 363(6426):274-276 (1993); Al-Fulaij et al., J. Pharmacol. Exp. Ther. 321(1):298-307 (2007)). These cells were starved in FCS-free DMEM medium for six hours before incubating them with 2 μM dopamine hydrochloride (Sigma Aldrich) together with each of the compound at 0.5 μM for three hours. The reporter activity was measured using Pierce™ Firefly Luciferase Glow Assay Kit (Thermo Fisher Scientific). Luminescence was monitored by SpectraMax® M5 Microplate Reader (Molecular Devices LLC). Among the PP2A activators tested, forskolin had a mild DRD2 inhibition activity (˜30%), but other compounds including iPAP1, iPAP5 (P-491313983), fingolimod and SMAP did not show inhibitory activities on DRD2 (FIG. 30).

### Example 17

### Flow Cytometric DNA Histogram of KOPT-K1 Cells Treated with PPZ, and iPAP1

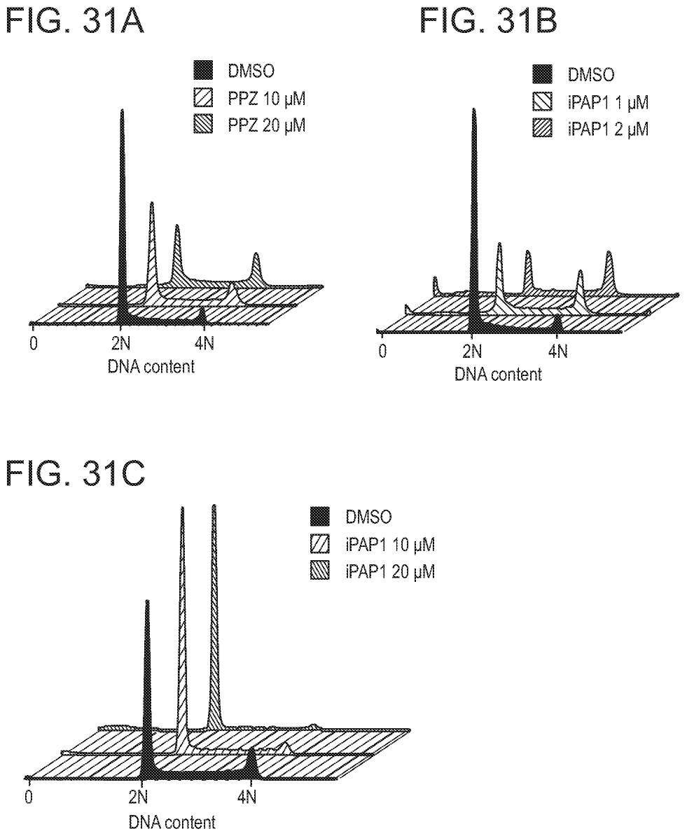

KOPT-K1 cells were treated with DMSO as control, PPZ or iPAP1 for 24 hours. Relative DNA content of cells in each of the samples was determined by measuring PI (propidium iodide) staining using flow cytometry. The results are illustrated in FIG. 31A-FIG. 31B. Treatment with PPZ (10 μM and 20 μM) or iPAP1 (1 μM and 2 μM) induced significant G2/M phase arrest in the cell cycle, as indicated with increased cells with 4N DNA content.

FIG. 31C is a flow cytometric DNA histogram that shows the cell cycle status of KOPT-K1 cells treated with DMSO as control or SMAP for 24 hours. Relative DNA content of cells in each of the samples was determined as described above. Treatment with SMAP (10 μM and 20 μM) induced significantly increased G0/G1 phase cells, and decreased cells in S phase and G2/M phase of the cell cycle, and thus the cells were arrested in G0/G1 phase rather than G2/M phase, indicating that the antiproliferative activities of SMAP are completely different from those of PPZ or iPAP1.

### Example 18

### Acetocarmine and Immunofluorescence Staining of KOPT-K1 Cells Treated with PPZ and iPAP1

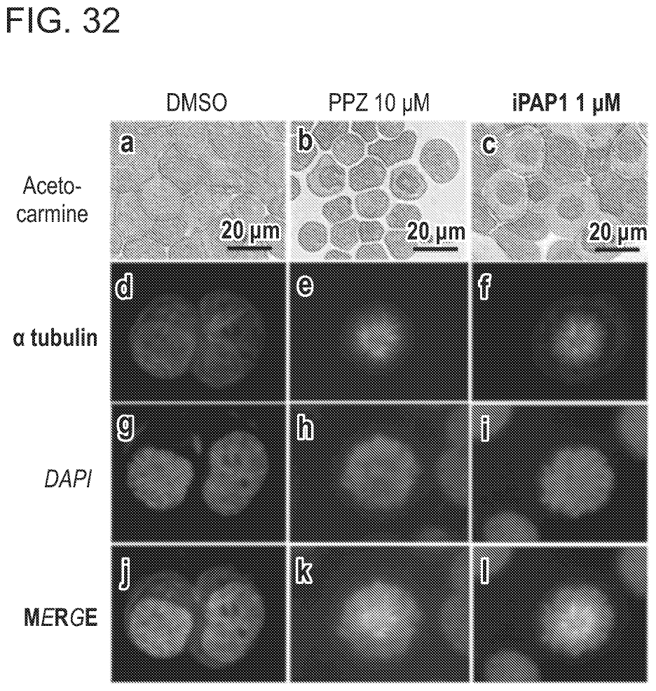

KOPT-K1 cells were treated with DMSO as control, PPZ at 10 μM or iPAP1 at 1 μM for 24 hours. For immunofluorescence staining, Alexa 647 (red)-anti-α tubulin antibody and DAPI were used to stain microtubules and DNA respectively. The results are illustrated in FIG. 32. PPZ and iPAP1 treatment induced prometaphase arrest in the cell cycle producing cells in which the spindle and associated microtubules exhibited monopolarity.

### Example 19

### PPZ and iPAP1 Effects on Gene Expression Levels in KPOT-K1 Cells

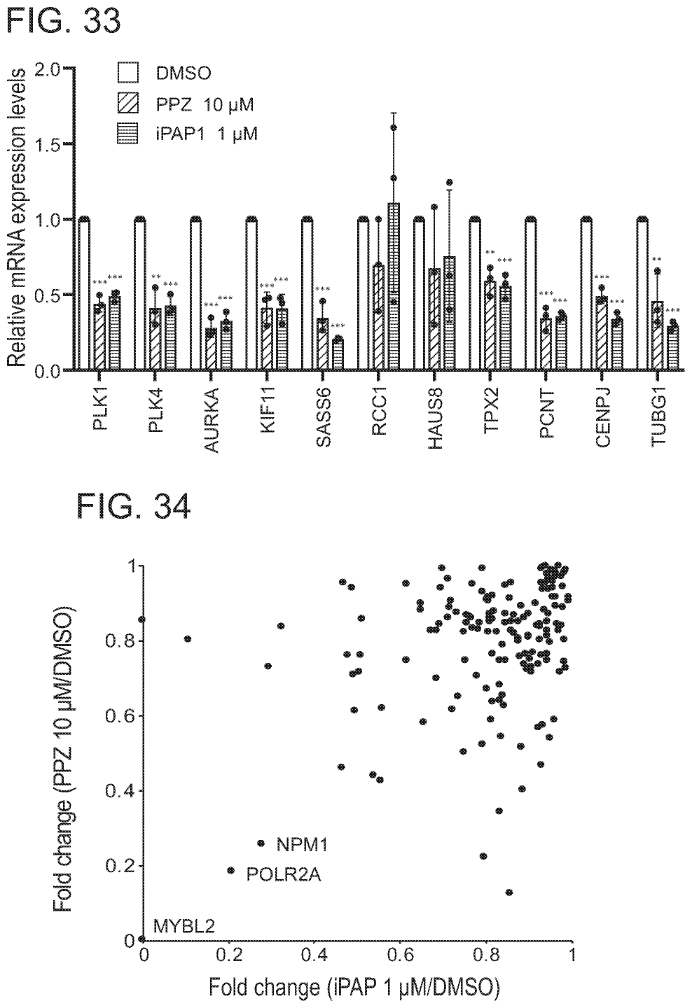

The relative mRNA expression of genes whose inducible CRISPR-cas9 knockout causes cell cycle arrest in prometaphase yielding spindle monopolarity (PLK1, PLK4, AURKA, KIF11, SASS6, RCC1, HAUS8, TPX2, PCNT, CENPJ and TUBG1) (McKinley et al., Dev. Cell 40:405-420 (2017)) was evaluated in KOPT-K2 cell lines. KOPT-K1 cells were treated with DMSO as control, PPZ at 10 μM or iPAP1 at 1 μM for 6 hours. The results are illustrated in FIG. 33. PPZ and iPAP1 treatment significantly down-regulated the expression levels of most of these genes.

### Example 20

### Phosphoproteomics Analysis Using KOPT-K1 Cells Treated with PPZ and iPAP1

To evaluate phosphopeptides in KOPT-K1 cells by phosphoproteomics analysis, cells were treated with PPZ at 10 μM or iPAP1 at 1 μM for 3 hours. In FIG. 34, fold changes of the counts of phosphopeptides in KOPT-K1 cells treated with PPZ and iPAP1 over control are shown in X and Y axis, respectively. The MYBL2 (MYB proto oncogene like 2) transcription factor was completely dephosphorylated at Ser241 after both PPZ and iPAP1 treatment, indicating that phopho-Ser241 in human MYBL2 was the substrate of PP2A that was most affected by activating PP2A with either of these drugs in KOPT-K1 T-ALL cells.

### Example 21

### Flow Cytometric DNA Histogram of KOPT-K1 Cells Treated with PPZ, and iPAP1 After MYBL2 Knockdown

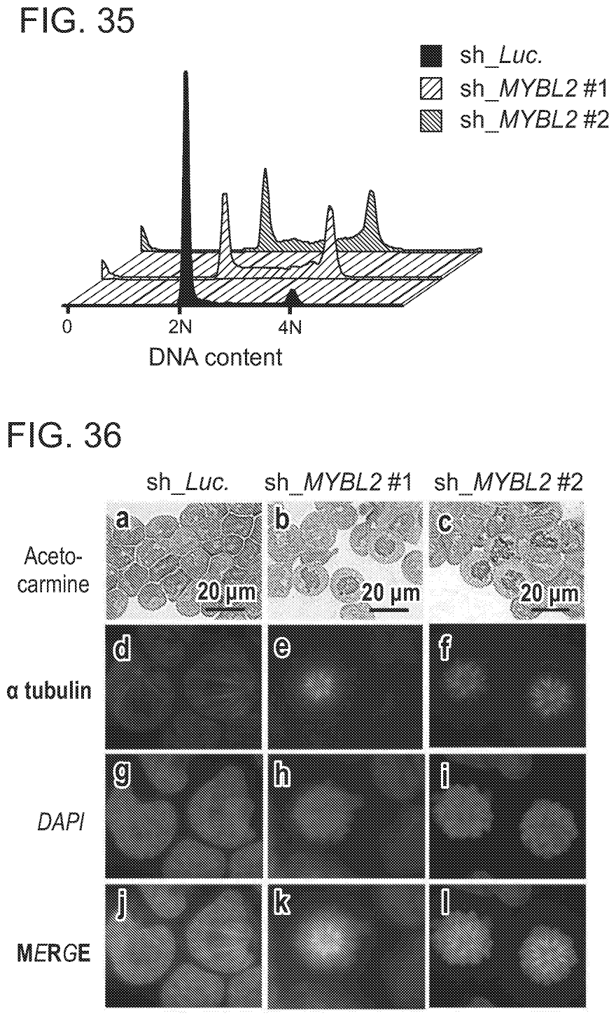

Cell cycle status of KOPT-K1 cells afterMYBL2 knockdown using gene specific shRNAs was determined by cellular DNA flow cytometry. Expression of shRNAs was induced by 3 μM doxycycline for 24 hours, and cellular DNA content of cells in each of the samples was measured by PI (propidium iodide) staining. The results are illustrated in FIG. 35. MYBL2 siRNA knockdown induced significant G2/M phase arrest with increased cells with 4N cellular DNA content of KOPT-K1 cells.

### Example 22

### Acetocarmine and Immunofluorescence Staining of KOPT-K1 Cells Treated with PPZ and iPAP1 After MYBL2 Knockdown

Expression of the shRNAs was induced by adding 3 μM doxycycline to the medium for 48 hours. For immunofluorescence staining, Alexa 647 (red)-anti-α tubulin antibody and DAPI were used to stain microtubules and DNA respectively. The results are illustrated in FIG. 36. Like PPZ and iPAP1 treatment, MYBL2 inactivation induced prometaphase arrest in the cell cycle with spindle and microtubule monopolarity.

### Example 23

### PPZ and iPAP1 Effects on Gene Expression Levels in KPOT-K1 Cells After MYBL2 Knockdown

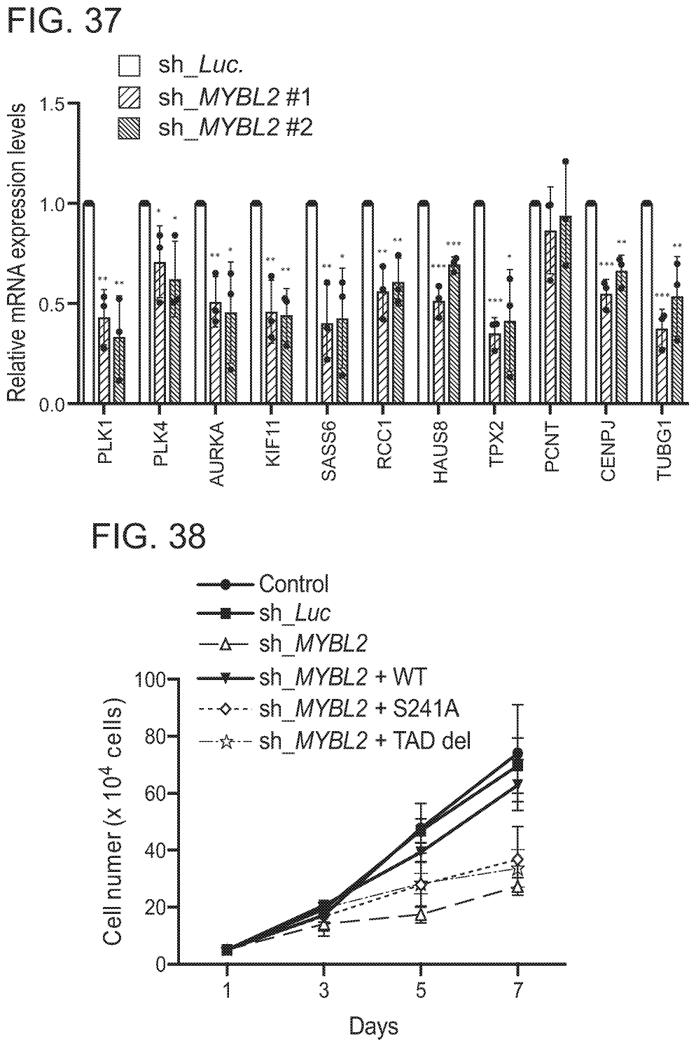

The relative mRNA expression levels of genes that are involved in spindle and microtubule monopolarity (PLK1, PLK4, AURKA, KIF11, SASS6, RCC1, HAUS8, TPX2, PCNT, CENPJ, and TUBG1) (McKinley et al., Dev. Cell 40:405-420 (2017)) were evaluated in KPOT-K1 cells treated with PZZ and iPAP1 after MYBL2 knockdown. MYBL2 was inactivated using gene specific doxycycline-inducible shRNAs. Induction of shRNAs for 24 hours with 3 μM doxycycline significantly down-regulated the expression levels of most of these genes. The results are illustrated in FIG. 37. Together with the results shown in FIG. 33, these results suggest that PPZ and its derivative iPAP1 induced prometaphase arrest in the cell cycle through dephosphorylation-mediated inactivation ofMYBL2 transcription factor in KOPT-K1 cells.

### Example 24

### Cell Proliferation of KPOT-K1 Cells with and without MYBL2 Knockdown

Cell proliferation curves of KOPT-K1 cells with or without MYBL2 inactivation using shRNA were determined and illustrated in FIG. 38. As shown previously, MYBL2 gene knockdown led to a significant reduction in cell growth rate. To test the hypothesis with phopho-Ser241 in human MYBL2, rescue experiments were conducted after MYBL2 shRNA-mediated knockdown. Rescue with wild-type (WT) MYBL2 successfully reverted the shMYBL2-induced suppression of cell growth, while a non-phosphorylatable alanine mutant MYBL2 S241A or transcriptional activation domain-deleted MYBL2 TAD-del could not rescue this phenotype, indicating that the transcriptional activation domain ofMYBL2, more specifically the S241 residue in this domain, is critically important for the function of this gene.

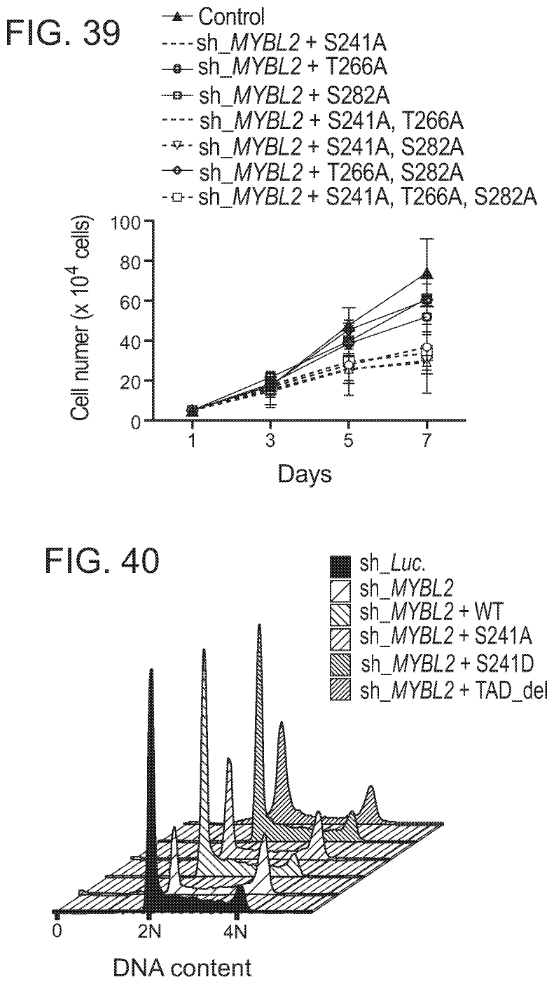

Rescue of cell growth effects of shRNA-mediated inactivation of MYBL2 was attempted with a series of non-phosphorylatable alanine mutants MYBL2 (S241A, T266A, S282A, S241A/T266A, S241A/S282A, T266A/S282A and S241A/T266A/S282A. o/e; overexpression). The results are illustrated in FIG. 39. Restoring MYBL2 expression using mutantMYBL2 harboring T266A, S282 or T266A/S282A successfully reverted the sh_MYBL2-induced suppression of cell growth, indicating that phosphorylation of these serines in the transcriptional activation domain of MYBL2 did not alter its ability to support cell cycle progression and cell growth. By contrast, mutant MYBL2 harboring S241A (S241A, S241A/T266A, S241A/S282A and S241A/T266A/S282A) could not rescue the cell growth phenotype, indicating that phospho-S241 in the transcriptional activation domain of MYBL2 was the only phosphorylation in the TAD that was important for rescuing the growth arrest of KOPTK1 cells induced by knockdown of MYBL2.

### Example 25

### Flow Cytometric DNA Histogram of KOPT-K1 After Inducible MYBL2 Knockdown

The cell cycle effects of MYBL2 shRNA knockdown were rescued by simultaneous overexpression of WTMYBL2 or mutantMYBL2 S241D (a phospho-mimic mutation). Expression of both the shRNAs and MYBL2 constructs were induced by 3 μM doxycycline for 24 hours, and relative cellular DNA content in each of the samples was measured by flow cytometric analysis of PI (propidium iodide) staining. An is histogram that shows the cell cycle status of KOPT-K1 cells after inducible MYBL2 knockdown using gene specific shRNA, demonstrating arrest of the cells in G2/M phase of the cell cycle with 4N DNA content. MYBL2 knockdown induced significant G2/M phase arrest in the cell cycle in KOPT-K1 cells. This G2/M phase cell cycle arrest was rescued by both WT MYBL2 and the phospho-mimic mutant MYBL2 S241D, but not by mutant MYBL2 S241A or the transcriptional activation domain deletion (MYBL2 TAD_del).

### Example 26

### Acetocarmine and Immunofluorescence Staining of KOPT-K1 Cells After MYBL2 Knockdown

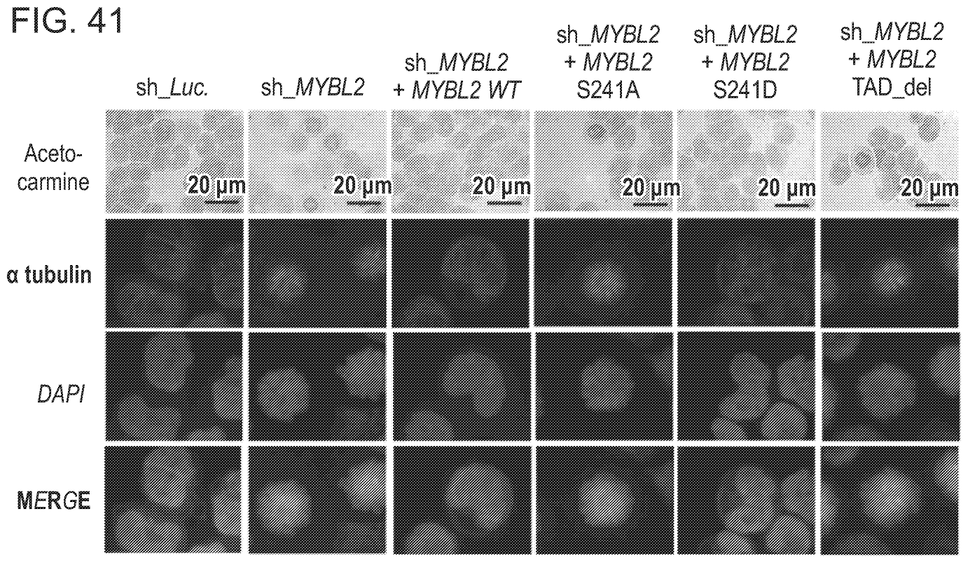

The rescue experiment included simultaneous overexpression of WT MYBL2 or series of mutant MYBL2 (S241A, S241D or transcriptional activation domain deletion (TAD_del)). Expression of shRNAs and MYBL2 were induced by 3 μM doxycycline for 24 hours. For immunofluorescence staining, Alexa 647 (red)-anti-α tubulin antibody and DAPI were used to stain microtubules and DNA respectively. The results are illustrated in FIG. 41. MYBL2 inactivation induced prometaphase arrest of the cell cycle with spindle and microtubule monopolarity was reverted by WT MYBL2 or mutant MYBL2 S241D, but not by mutant MYBL2 S241A or transcriptional activation domain deletion (TAD_del). Thus, the MYBL2 241S to 241D mutation produced a MYBL2 protein that mimics phospho-MYBL2 241S, but is not insensitive to the effects of PP2A phosphatase activation.

### Example 27

### IC₅₀ Values for PPZ and iPAP1 in KOPT-K1 Cells with the Phospho-Mimic Aspartic Acid Mutant Forms of MYBL2

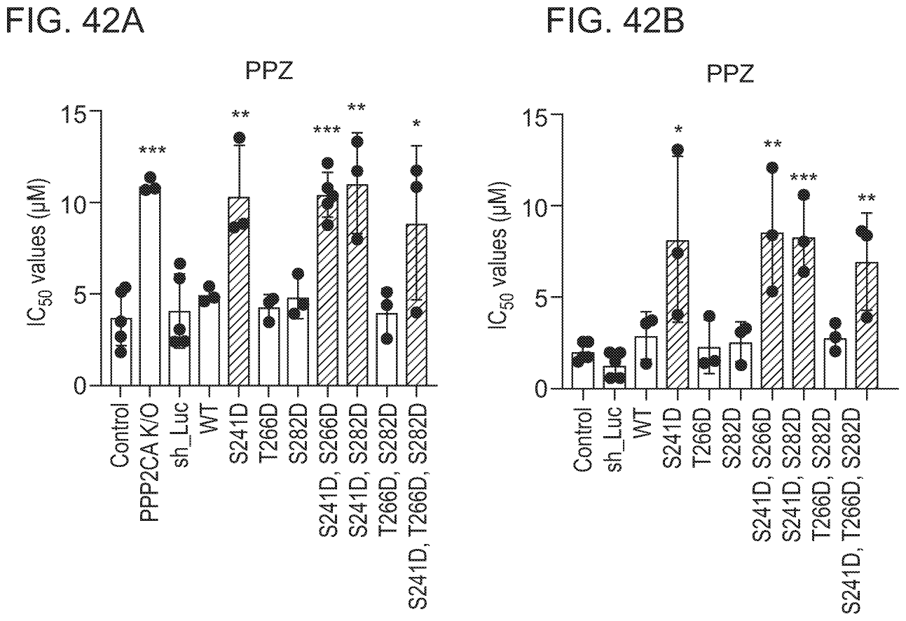

IC₅₀ values for PPZ and iPAP1 in KOPT-K1 cells with the phospho-mimic aspartic acid mutant forms of MYBL2 were determined. The results are illustrated in FIG. 42A-FIG. 42B. In shMYBL2 knockout cells, the overexpression of mutant MYBL2 harboring S241D (S241D, S241D/T266D, S241D/S282D and S241D/T266D/S282D) conferred resistance to PPZ or iPAP1 treatment in KOPT-K1 cells. However, the T266D, S282D or T266D/S282D expressing cells still showed G2/M arrest in response to treatment with PPZ or iPAP1, indicating that PP2A-induced dephosphorylation of phospho-S241 of MYBL2 is the sole cause of the prometaphase arrest of KOPT-K1 cells in G2/M phase of the cell cycle due to activation of PP2A by treatment with both PPZ and iPAP1.

### Example 28

### Relative Activities of Promoters for PLK1 and KIF11

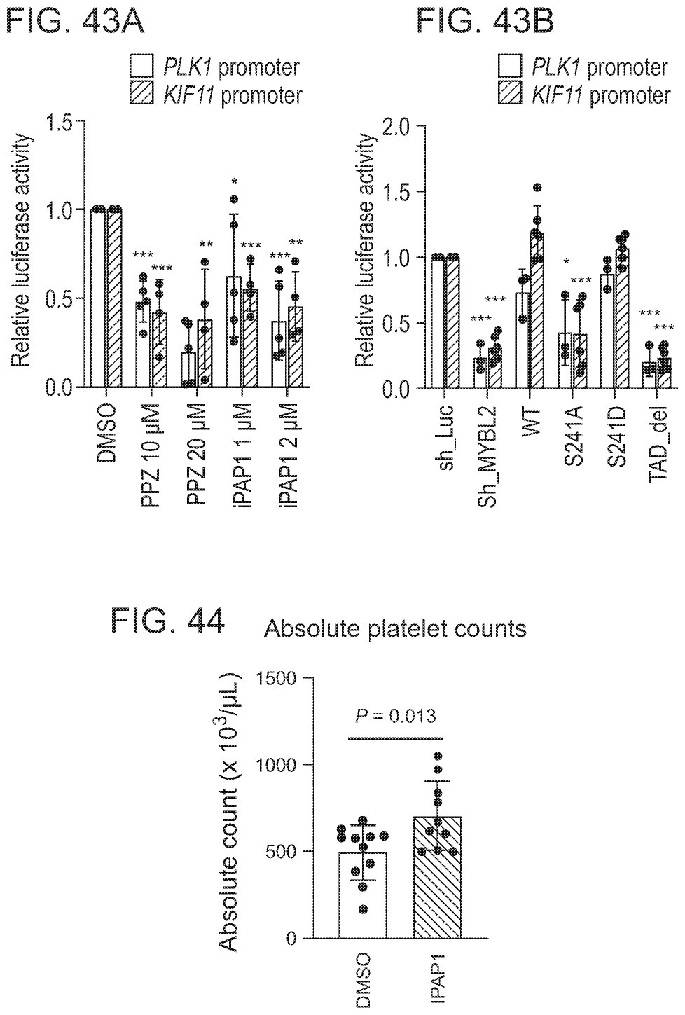

The relative activities of promoters for two representative MYBL2 target genes, PLK1 and KIF11, which each cause cell cycle arrest in prometaphase yielding spindle monopolarity (McKinley et al., Dev. Cell, 40:405-420 (2017)), were determined. HEK293T cells were transiently transfected with a vector expressing luciferase under control of either the PLK1 promoter or the KIF11 promoter. The activities of the promoters were measured by detecting luminescence. The results are illustrated in FIG. 43A-FIG. 43B. EndogenousMYBL2 was knocked down using gene specific shRNAs, then its expression was restored by simultaneous overexpression of WT MYBL2 or series of mutant MYBL2 constructs (S241A, S241D or transcriptional activation domain deletion (TAD_del)). Expression of both shRNAs and MYBL2 constructs were induced by 3 μM doxycycline for 24 hours. MYBL2 knockdown induced downregulation of these promoters, and the luciferase activity of both was upregulated by WT MYBL2 or mutant MYBL2 S241D, but not by mutant MYBL2 S241A or transcriptional activation domain deletion (TAD_del). These results underscore the importance of S241 phosphorylation for the function of MYBL2 as a transcription factor. Phospo-MYBL2-ser241 is required for MYBL2 to activate the expression of genes like PLK1 and KIFT1 that are required for cells to move out of the stage of spindle and microtubule monopolarity and progress through mitosis (see, McKinley et al., Dev. Cell 40:405-420 (2017)).

### Example 29

### Tubulin Polymerization Assay

Tubulin polymerization assay was conducted using Tubulin polymerization assay>99% pure tubulin, fluorescence-based kit (BK011P, CYTOSKELETON, INC) according to the manufacturer's instruction. Briefly, free tubulin (2 mg/mL) in buffer supplemented with 1 mM GTP and 15% glycerol was employed. Then, the test compounds were added to tubulin solution and changes in the fluorescence intensity (ex=370 nm, em=445 nm) were measured by kinetic reading at 37° C. using SpectraMax® M5 Microplate reader (Molecular Devices LLC).

### Example 30

### Absolute Platelet Counts in Peripheral Blood

The peripheral blood platelet counts in C57BL/6J mice treated with either by DMSO or iPAP1 at 80 mg/kg/day intraperitoneally for seven consecutive days, were measured. The results, shown in FIG. 44, demonstrate that iPAP1 treatment significantly increased the platelet counts in the blood (P=0.013 by two-sided student's t-test). Other blood cell counts were unchanged in treated compared to control mice. iPAP1 treatment of mice increased the platelet count, due to effects of the compound on endoreduplication of megakaryocytes.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of treating a cancer, comprising administering to a subject in need thereof a therapeutically effective amount of a perphenazine (PPZ) analog which has a structure represented by formula I or II: 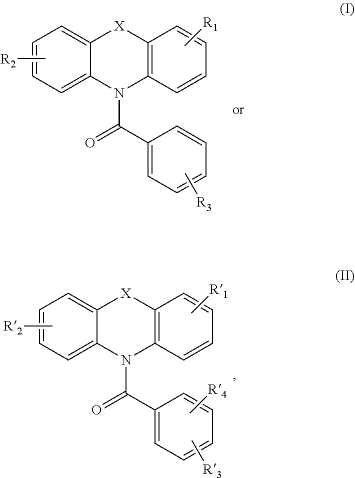
wherein X is O or S;
R₁ and R₂ are independently H, halo (e.g., Cl or F), NO₂ or CN;
R₃ is C1-C2 alkyl or methoxy;
R′₁ and R′₂ are independently H, halo, NO₂ or CN;
R′₃ and R′₄ are independently halo, NO₂, CN, C1-C2 alkyl, methoxy, ethoxy, propoxy, isopropoxy, butoxy or benzyloxy; or R′₃ and R′₄ together with the atoms to which they are bound form a 6-membered aryl or 6-membered heteroaryl group, or a pharmaceutically acceptable salt thereof.

2. A method of treating a cancer, comprising administering to a subject in need thereof a therapeutically effective amount of a perphenazine (PPZ) analog or a pharmaceutically acceptable salt thereof, identified by selecting for optimal PP2A activity and a lack of inhibition of the dopamine D2 receptor.

3. The method of any one of claims 1-2, wherein the PPZ analog has anyone of structures IPAP1 to iPAP24: 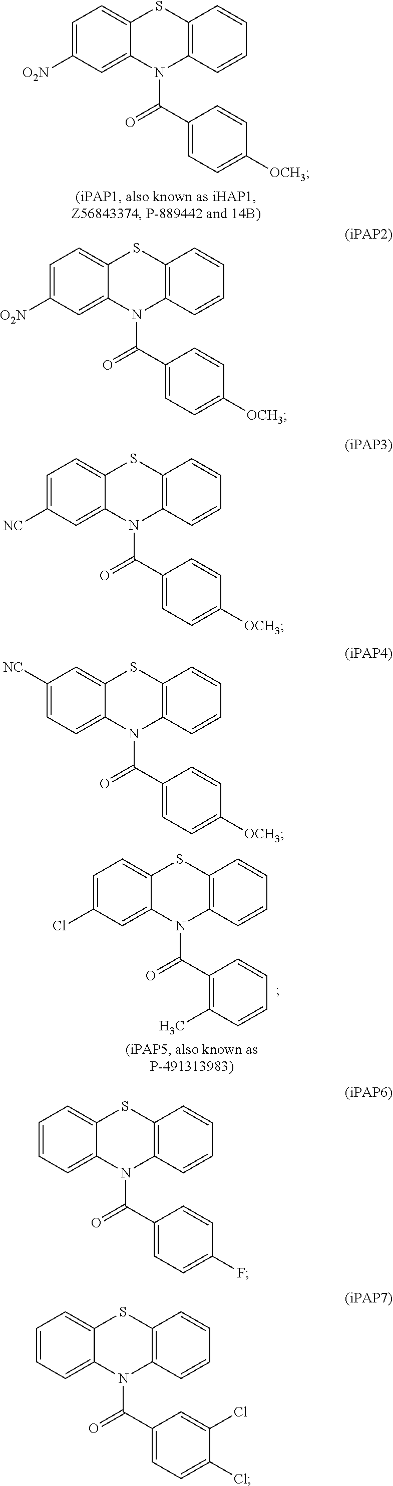 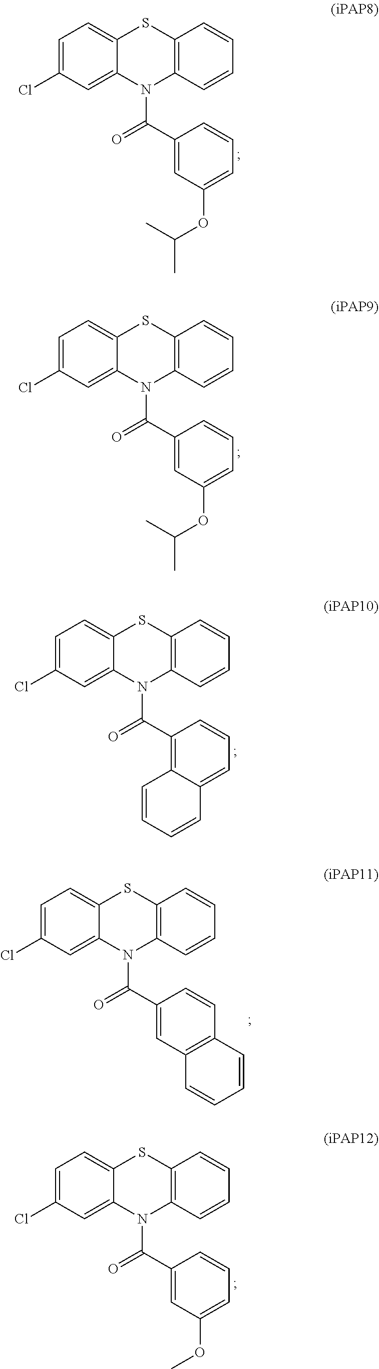 [SMILES: CC(C)Oc1cccc(C(=O)N2c3ccccc3Sc3ccc(Cl)cc32)c1 ; CC(C)Oc1cccc(C(=O)N2c3ccccc3Sc3ccc(Cl)cc32)c1 ; O=C(c1cccc2ccccc12)N1c2ccccc2Sc2ccc(Cl)cc21 ; O=C(c1ccc2ccccc2c1)N1c2ccccc2Sc2ccc(Cl)cc21 ; COc1cccc(C(=O)N2c3ccccc3Sc3ccc(Cl)cc32)c1] 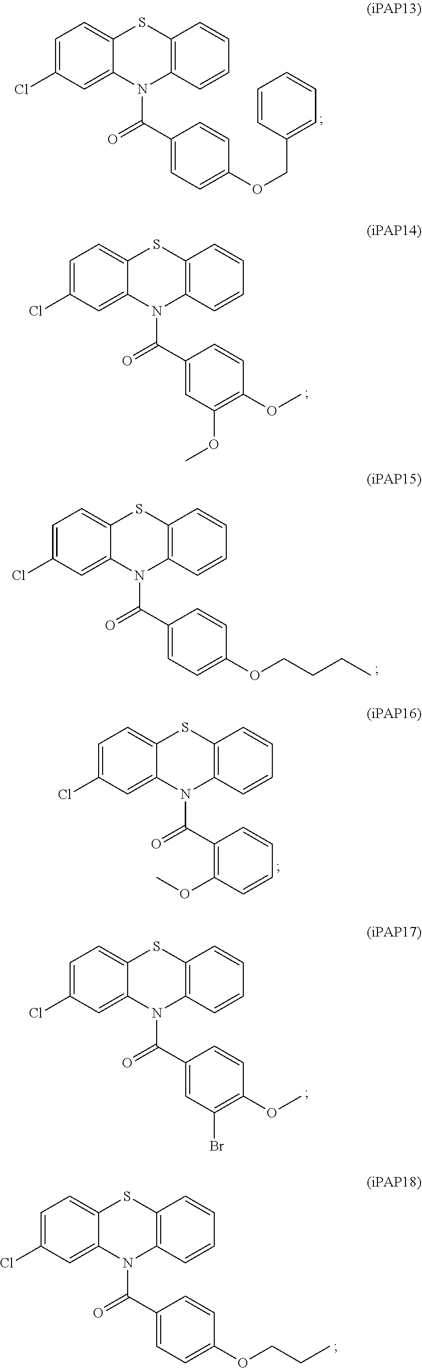 [SMILES: O=C(c1ccc(OCc2ccccc2)cc1)N1c2ccccc2Sc2ccc(Cl)cc21 ; COc1ccc(C(=O)N2c3ccccc3Sc3ccc(Cl)cc32)cc1OC ; CCCCOc1ccc(C(=O)N2c3ccccc3Sc3ccc(Cl)cc32)cc1 ; COc1ccccc1C(=O)N1c2ccccc2Sc2ccc(Cl)cc21 ; COc1ccc(C(=O)N2c3ccccc3Sc3ccc(Cl)cc32)cc1Br ; CCCOc1ccc(C(=O)N2c3ccccc3Sc3ccc(Cl)cc32)cc1] 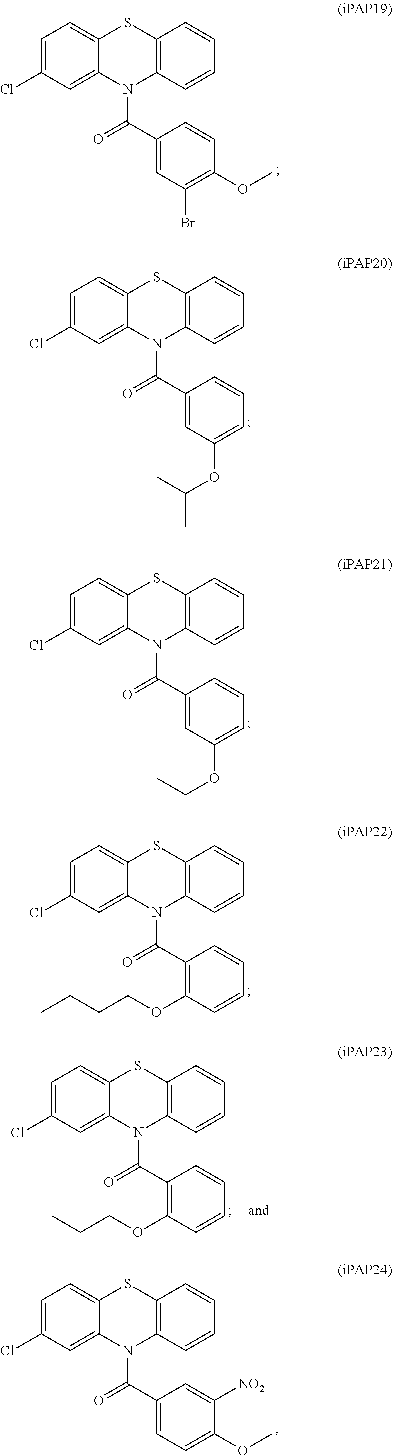 [SMILES: COc1ccc(C(=O)N2c3ccccc3Sc3ccc(Cl)cc32)cc1Br ; CC(C)Oc1cccc(C(=O)N2c3ccccc3Sc3ccc(Cl)cc32)c1 ; CCOc1cccc(C(=O)N2c3ccccc3Sc3ccc(Cl)cc32)c1 ; CCCCOc1ccccc1C(=O)N1c2ccccc2Sc2ccc(Cl)cc21 ; CCCOc1ccccc1C(=O)N1c2ccccc2Sc2ccc(Cl)cc21 ; COc1ccc(C(=O)N2c3ccccc3Sc3ccc(Cl)cc32)cc1[N+](=O)[O-]]
or a pharmaceutically acceptable salt thereof.

4. The method of any one of claims 1-2, wherein the PPZ analog is: 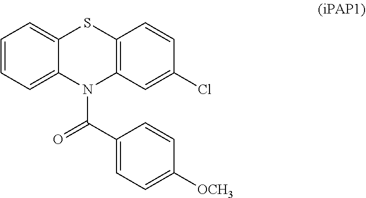 [SMILES: COc1ccc(C(=O)N2c3ccccc3Sc3ccc(Cl)cc32)cc1]
or a pharmaceutically acceptable salt thereof.

5. The method of any one of claims 1-2, wherein the PPZ analog is: 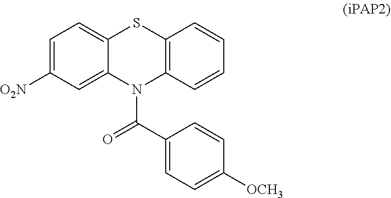 [SMILES: COc1ccc(C(=O)N2c3ccccc3Sc3ccc([N+](=O)[O-])cc32)cc1]
or a pharmaceutically acceptable salt thereof.

6. The method of any one of claims 1-2, wherein the PPZ analog is: 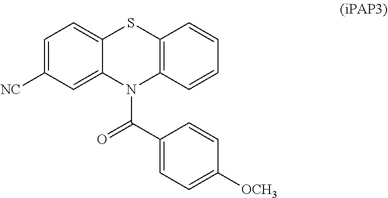 [SMILES: COc1ccc(C(=O)N2c3ccccc3Sc3ccc(C#N)cc32)cc1]
or a pharmaceutically acceptable salt thereof.

7. The method of any one of claims 1-2, wherein the PPZ analog is: 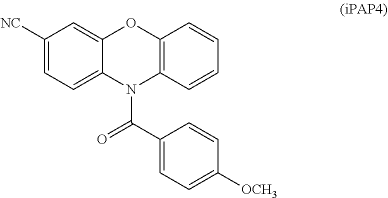 [SMILES: COc1ccc(C(=O)N2c3ccccc3Oc3cc(C#N)ccc32)cc1]
or a pharmaceutically acceptable salt thereof.

8. The method of any one of claims 1-2, wherein the PPZ analog is: 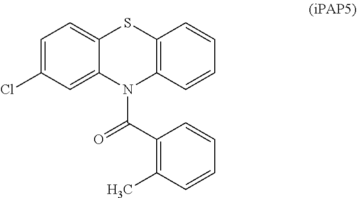 [SMILES: Cc1ccccc1C(=O)N1c2ccccc2Sc2ccc(Cl)cc21]
or a pharmaceutically acceptable salt thereof.

9. The method of any one of claims 1-2, wherein the cancer is a hematological cancer.

10. The method of claim 9, wherein the cancer is T-cell acute lymphoblastic leukemia (T-ALL), T-cell non-Hodgkin's lymphoma, acute myeloid leukemia (AML), chronic eosinophilic leukemia, chronic myeloid leukemia, B-cell acute lymphocytic leukemia (B-ALL), B-cell non-Hodgkin lymphoma, plasma cell myeloma, or Hodgkin lymphoma.

11. The method of claim 10, wherein the cancer is T-cell acute lymphoblastic leukemia (T-ALL).

12. The method of any one of claims 1-2, wherein the cancer is neuroblastoma, small cell lung carcinoma, lung adenocarcinoma and squamous cell carcinoma, gastric carcinoma, glioblastoma, primitive neuroectodermal tumor, meningioma, esophageal squamous cell carcinoma, endometrial carcinoma, medulloblastoma, melanoma, head and neck squamous cell carcinoma, pleural epithelioid mesothelioma, renal cell carcinoma, breast carcinoma, pancreatic ductal adenocarcinoma, ovarian carcinoma, osteosarcoma, or colon carcinoma.

13. The method of any one of claims 1-2, wherein the method further comprises administering the therapeutically effective amount of the compound of formula I or II, or a related PPZ analog lacking dopamine receptor D2 inhibitory activity, or a pharmaceutically acceptable salt thereof, to the subject, in combination with a therapeutically effective amount of an additional chemotherapeutic agent.

14. The method of claim 13, wherein the chemotherapeutic agent comprises vincristine, VP16, an anthracycline such as daunorubicin, or an epipodophyllotoxin.

15. The method of claim 13, wherein the chemotherapeutic agent is nelarabine, methotrexate (MTX), or polyethylene glycol (PEG)-asparaginase.

16. The method of claim 13, wherein the chemotherapeutic agent is a gamma-secretase inhibitor.

17. The method of claim 16, wherein the gamma-secretase inhibitor is BMS-906024, BMS-986115, N-[N-(3,5-Difluorophenacetyl)-L-alanyl]-S-phenylglycine t-butyl ester (DAPT), LY90000, LY3039478, LY411575, MK-0752, PF-3084014, or RO4929097.

18. The method of claim 13, wherein the chemotherapeutic agent is anti-Notch monoclonal antibody (anti-Notch1, anti-Notch2, anti-delta-like protein (DLL) 4).

19. The method of claim 18, wherein the anti-Notch monoclonal antibody is OMP52M51, OMP59RPuPP5, and REGN421.

20. The method of claim 13, wherein the chemotherapeutic agent is an anti-Notch soluble notch protein.

21.-75. (canceled)
